(19) United States
(12) Patent Application Publication
(10) Pub. No.: US 2024/0415884 A1
(43) Pub. Date: Dec. 19, 2024

(54) MOLECULES THAT BIND TO CD276 POLYPEPTIDES

(71) Applicant: University of Pittsburgh of the Commonwealth System of Higher Education, Pittsburgh, PA (US)

(72) Inventors: Dimiter Stanchev Dimitrov, Pittsburgh, PA (US); Xianglei Liu, Pittsburgh, PA (US); Wei Li, Pittsburgh, PA (US); John W. Mellors, Pittsburgh, PA (US); Dusan Baek, Pittsburgh, PA (US); Zehua Sun, Pittsburgh, PA (US)

(21) Appl. No.: 18/700,535

(22) PCT Filed: Jan. 31, 2023
(86) PCT No.: PCT/US2023/011929

§ 371 Date: Apr. 11, 2024

Related U.S. Application Data
(60) Provisional application No. 63/305,525, filed on Feb. 1, 2022.

(51) Int. Cl. A61K 35/17; A61K 39/00; A61K 45/06; A61P 35/00; C07K 16/28

(52) U.S. Cl. CPC A61K 35/17; A61K 39/4611; A61K 39/4631; A61K 39/464411; A61K 45/06; A61P 35/00; C07K 16/2827; A61K 2239/13; A61K 2239/17; A61K 2239/21; C07K 2317/56

(57) ABSTRACT

This document provides methods and materials involved in binding a binder (e.g., an antibody, antigen binding fragment, antibody domain, CAR, cell engager, and/or ADC) to a CD276 polypeptide. For example, binders (e.g., antibodies, antigen binding fragments, antibody domains, CARs, cell engagers, and/or ADCs) that bind to a CD276 polypeptide and methods and materials for using one or more such binding molecules to treat a mammal (e.g., a human) having cancer are provided.

### CROSS-REFERENCE TO RELATED APPLICATIONS

This application is a National Stage application under 35 U.S.C. § 371 of International Application No. PCT/US2023/011929, having an International Filing Date of Jan. 31, 2023, which claims the benefit of U.S. Provisional Application Ser. No. 63/305,525, filed Feb. 1, 2022. The disclosure of the prior application is considered part of (and is incorporated by reference in) the disclosure of this application.

### SEQUENCE LISTING

This application contains a Sequence Listing that has been submitted electronically as an XML file named “48881-0022W01_SL.xml.” The XML file, created on May 19, 2023, is 518,483 bytes in size. The material in the XML file is hereby incorporated by reference in its entirety.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a CD276 polypeptide. For example, this document provides binders (e.g., antibodies, antigen binding fragments, antibody domains, CARs, cell engagers, or ADCs) that bind to a CD276 polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a CD276 polypeptide and methods and materials for using such cells to treat cancer.

### 2. Background Information

CD276, also known as B7-H3, is a member of the B7 family of immune checkpoint molecules. Overexpression of CD276 by cancer cells can indicate poor patient outcomes. Omburtamab and enoblituzumab are two humanized monoclonal anti-CD276 IgG1κ antibodies that were developed for the treatment of cancer.

### SUMMARY

This document provides methods and materials involved in binding a molecule (e.g., an antibody, an antigen binding fragment, an antibody domain, a CAR, a cell engager, or an ADC) to a CD276 polypeptide. For example, this document provides binders (e.g., antibodies, antigen binding fragments, antibody domains, CARs, cell engagers, or ADCs) that bind to a CD276 polypeptide and methods and materials for using one or more such binders to treat a mammal (e.g., a human) having cancer.

This document also provides cells (e.g., host cells) designed to express one or more binders (e.g., antibodies, antigen binding fragments, antibody domains, CARs, or cell engagers) having the ability to bind to a CD276 polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to a CD276 polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human CD276 polypeptide as set forth in SEQ ID NO:81 or SEQ ID NO:82 or SEQ ID NO:471 (see, e.g., FIG. 1).

In some cases, a single set of three complementarity-determining regions (CDRs) of an antibody domain (e.g., a VH domain) provided herein (e.g., SEQ ID NOs:49-51; SEQ ID NOs:57-59; SEQ ID NOs:65-67; SEQ ID NOs:73-75; or SEQ ID NOs:529-531; SEQ ID NOs:537-539) or two sets of three CDRs of an antigen binding fragment provided herein (e.g., SEQ ID NOs:1-3 and 9-11; SEQ ID NOs:17-19 and 25-27; SEQ ID NOs:33-35 and 41-43; or SEQ ID NOs:473-475 and 481-483) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target CD276⁺ cells (e.g., CD276⁺ tumor cells and/or CD276⁺ tumor vasculature), can be engineered into an antibody structure that includes an Fc region to create antibodies having the ability to target CD276⁺ cells (e.g., CD276⁺ tumor cells and/or CD276⁺ tumor vasculature) and induce antibody-dependent cell-mediated cytotoxicity (ADCC) against the target CD276⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target CD276⁺ cells (e.g., CD276⁺ tumor cells and/or CD276⁺ tumor vasculature) and induce one or more immune responses (e.g., T cell immune responses and/or ADCC using a cell engager in the absence of an Fc-containing antibody) against the target CD276⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., CD276⁺ cancer cells) or cancer vasculature (e.g., CD276⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a CD276⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to a CD276 polypeptide. For example, cells such as T cells (e.g., CTLs), stem cells (e.g., induced pluripotent stem cells), or NK cells can be engineered to express one or more CARs having the ability to bind to a CD276 polypeptide. Such cells (e.g., CD276-specific CAR⁺ T cells or NK cells) can be used to treat cancer.

In some cases, a binder (e.g., an antibody, antigen binding fragment, and/or antibody domain) provided herein can be used to detect the presence or absence of a CD276 polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains CD276⁺ cells (e.g., CD276⁺ cancer cells). Having the ability to detect the presence or absence of a CD276 polypeptide (e.g., CD276⁺ cancer cells) can allow clinicians, health professionals, and patients to make better decisions about possible treatment options. For example, detection of CD276⁺ cancer cells within a mammal can allow clinicians, health professionals, and patients to select an appropriate anti-cancer treatment that targets the CD276⁺ cancer cells. Such treatments that targets the CD276⁺ cancer cells can include administration of an anti-CD276 antibody such as Enoblituzumab and/or one or more of the binders described herein having the ability to bind to a CD276 polypeptide and/or administration of one or more cells (e.g., CD276-specific CAR⁺ T cells or NK cells) designed to express a binder described herein.

In some cases, a binder (e.g., an antibody, antigen binding fragment, and/or antibody domain) provided herein can be used to target CD276⁺ solid cancers and/or CD276⁺ cells within a tumor micro-environments.

In some cases, a binder (e.g., an antibody, antigen binding fragment, and/or antibody domain) provided herein can bind to an epitope of a CD276 polypeptide that is within an FG loop located between an IgV and IgC domain of the CD276 polypeptide. In such cases, a binder provided herein can inhibit the activity of a CD276 polypeptide.

In some cases, an antibody domain (e.g., a VH domain) provided herein can have a small size of less than 20 kDa (e.g., about 15 kDa) and can infiltrate tumors more efficiently than larger sized binders (e.g., full IgG's). In some cases, an antibody domain (e.g., a VH domain) provided can target one or more cryptic epitopes that are otherwise inaccessible to larger sized binders (e.g., full IgG's).

In some cases, a binder (e.g., an antibody, antigen binding fragment, and/or antibody domain) provided herein can exhibit little, or no, aggregation (e.g., self-aggregation). For example, a binder (e.g., an antibody, antigen binding fragment, and/or antibody domain) provided herein can be resistant to aggregation (e.g., self-aggregation). In some cases, a binder (e.g., an antibody, antigen binding fragment, and/or antibody domain) provided herein can be a binder that does not exhibit aggregation after three months incubation at 37° C.

In general, one aspect of this document features an antibody comprising (or consisting essentially of, or consisting of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antibody can comprise the ability to bind to SEQ ID NO:81 or SEQ ID NO:82 or SEQ ID NO:471. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can comprise the heavy chain variable domain or region of the (iii). The heavy chain variable domain or region can comprise an amino acid sequence having at least 90 percent identity to the amino acid sequence set forth in SEQ ID NO:40. The antibody can comprise the light chain variable domain or region of the (iii). The light chain variable domain or region can comprise an amino acid sequence having at least 90 percent identity to the amino acid sequence set forth in SEQ ID NO:48. The antibody can comprise the heavy chain variable domain or region of the (viii). The heavy chain variable domain or region can comprise an amino acid sequence having at least 90 percent identity to the amino acid sequence set forth in SEQ ID NO:480. The antibody can comprise the light chain variable domain or region of the (viii). The light chain variable domain or region can comprise an amino acid sequence having at least 90 percent identity to the amino acid sequence set forth in SEQ ID NO:488. The antibody can comprise the heavy chain variable domain or region of the (iv). The heavy chain variable domain or region can comprise an amino acid sequence having at least 90 percent identity to the amino acid sequence set forth in SEQ ID NO:56. The antibody can comprise the heavy chain variable domain or region of the (v). The heavy chain variable domain or region can comprise an amino acid sequence having at least 90 percent identity to the amino acid sequence set forth in SEQ ID NO:64. The antibody can comprise the heavy chain variable domain or region of the (vi). The heavy chain variable domain or region can comprise an amino acid sequence having at least 90 percent identity to the amino acid sequence set forth in SEQ ID NO:72. The antibody can comprise the heavy chain variable domain or region of the (vii). The heavy chain variable domain or region can comprise an amino acid sequence having at least 90 percent identity to the amino acid sequence set forth in SEQ ID NO:80. The antibody can comprise the heavy chain variable domain or region of the (ix). The heavy chain variable domain or region can comprise an amino acid sequence having at least 90 percent identity to the amino acid sequence set forth in SEQ ID NO:536. The antibody can comprise the heavy chain variable domain or region of the (x). The heavy chain variable domain or region can comprise an amino acid sequence having at least 90 percent identity to the amino acid sequence set forth in SEQ ID NO:544. The antibody can be a monoclonal antibody. The antibody can be an scFv antibody.

In another aspect, this document features an antigen binding fragment comprising (or consisting essentially of, or consisting of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antigen binding fragment can comprise the ability to bind to SEQ ID NO:81 or SEQ ID NO:82 or SEQ ID NO:471. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can comprise the heavy chain variable domain or region of the (iii). The heavy chain variable domain or region can comprise an amino acid sequence having at least 90 percent identity to the amino acid sequence set forth in SEQ ID NO:40. The antigen binding fragment can comprise the light chain variable domain or region of the (iii). The light chain variable domain or region can comprise an amino acid sequence having at least 90 percent identity to the amino acid sequence set forth in SEQ ID NO:48. The antigen binding fragment can comprise the heavy chain variable domain or region of the (viii). The heavy chain variable domain or region can comprise an amino acid sequence having at least 90 percent identity to the amino acid sequence set forth in SEQ ID NO:480. The antigen binding fragment can comprise the light chain variable domain or region of the (viii). The light chain variable domain or region can comprise an amino acid sequence having at least 90 percent identity to the amino acid sequence set forth in SEQ ID NO:488. The antigen binding fragment can comprise the heavy chain variable domain or region of the (iv). The heavy chain variable domain or region can comprise an amino acid sequence having at least 90 percent identity to the amino acid sequence set forth in SEQ ID NO:56. The antigen binding fragment can comprise the heavy chain variable domain or region of the (v). The heavy chain variable domain or region can comprise an amino acid sequence having at least 90 percent identity to the amino acid sequence set forth in SEQ ID NO:64. The antigen binding fragment can comprise the heavy chain variable domain or region of the (vi). The heavy chain variable domain or region can comprise an amino acid sequence having at least 90 percent identity to the amino acid sequence set forth in SEQ ID NO:72. The antigen binding fragment can comprise the heavy chain variable domain or region of the (vii). The heavy chain variable domain or region can comprise an amino acid sequence having at least 90 percent identity to the amino acid sequence set forth in SEQ ID NO:80. The antibody can comprise the heavy chain variable domain or region of the (ix). The heavy chain variable domain or region can comprise an amino acid sequence having at least 90 percent identity to the amino acid sequence set forth in SEQ ID NO:536. The antibody can comprise the heavy chain variable domain or region of the (x). The heavy chain variable domain or region can comprise an amino acid sequence having at least 90 percent identity to the amino acid sequence set forth in SEQ ID NO:544. The antigen binding fragment can be monoclonal. The antigen binding fragment can be an Fab.

In another aspect, this document features an antibody domain comprising (or consisting essentially of, or consisting of) (i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antibody domain can comprise the ability to bind to SEQ ID NO:81 or SEQ ID NO:82 or SEQ ID NO:471. The antibody domain can comprise the heavy chain variable domain or region of the (i). The heavy chain variable domain or region can comprise an amino acid sequence having at least 90 percent identity to the amino acid sequence set forth in SEQ ID NO:56. The antibody domain can comprise the heavy chain variable domain or region of the (ii). The heavy chain variable domain or region can comprise an amino acid sequence having at least 90 percent identity to the amino acid sequence set forth in SEQ ID NO:64. The antibody domain can comprise the heavy chain variable domain or region of the (iii). The heavy chain variable domain or region can comprise an amino acid sequence having at least 90 percent identity to the amino acid sequence set forth in SEQ ID NO:72. The antibody domain can comprise the heavy chain variable domain or region of the (iv). The heavy chain variable domain or region can comprise an amino acid sequence having at least 90 percent identity to the amino acid sequence set forth in SEQ ID NO:80. The antibody can comprise the heavy chain variable domain or region of the (v). The heavy chain variable domain or region can comprise an amino acid sequence having at least 90 percent identity to the amino acid sequence set forth in SEQ ID NO:536. The antibody can comprise the heavy chain variable domain or region of the (vi). The heavy chain variable domain or region can comprise an amino acid sequence having at least 90 percent identity to the amino acid sequence set forth in SEQ ID NO:544. The antibody domain can be monoclonal. The antibody domain can be a VH domain.

In another aspect, this document features a chimeric antigen receptor comprising (or consisting essentially of, or consisting of) an antigen binding domain, a hinge, a transmembrane domain, and one or more signaling domains, wherein the antigen binding domain comprises an antibody, an antigen-binding fragment, or an antibody domain,
wherein the antibody comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antigen binding domain can comprise a scFv having the ability to bind to a CD276 polypeptide. The antigen binding domain can comprise a VH domain having the ability to bind to a CD276 polypeptide. The hinge can comprise a hinge set forth in FIG. 13B. The transmembrane domain can comprise a transmembrane domain set forth in FIG. 14. The chimeric antigen receptor can comprise one or more signaling domains set forth in FIG. 15.

In another aspect, this document features a cell comprising a chimeric antigen receptor of the preceding paragraph. The cell can be a T cell, a stem cell, or an NK cell.

In another aspect, this document features a cell engager comprising (or consisting essentially of, or consisting of) a first antigen binding domain, a linker, and a second antigen binding domain, wherein the first antigen binding domain comprises an antibody, an antigen-binding fragment, or an antibody domain,
wherein the antibody comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The first antigen binding domain can comprise a scFv having the ability to bind to a CD276 polypeptide. The first antigen binding domain can comprise a VH domain having the ability to bind to a CD276 polypeptide. The linker can comprise a linker set forth in FIG. 10 or FIG. 13B. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 16. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 17. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 17.

In another aspect, this document features a nucleic acid comprising (or consisting essentially of, or consisting of) a nucleic acid sequence encoding at least part of (or the full length of) an antibody, an antigen-binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The nucleic acid sequence can encode the heavy chain variable domain or region of any one of (a-i)-(a-x). The nucleic acid sequence can encode the light chain variable domain or region of any one of the (a-i)-(a-iii) and (a-viii). The nucleic acid can be a viral vector. The nucleic acid can be a phagemid.

In another aspect, this document features a nucleic acid comprising (or consisting essentially of, or consisting of) a nucleic acid sequence encoding a chimeric antigen receptor or a cell engager, wherein the chimeric antigen receptor comprises (or consists essentially of, or consists of) an antigen binding domain, a hinge, a transmembrane domain, and one or more signaling domains, wherein the antigen binding domain comprises an antibody, an antigen-binding fragment, or an antibody domain, wherein the cell engager comprises (or consists essentially of, or consists of) a first antigen binding domain, a linker, and a second antigen binding domain, wherein the first antigen binding domain comprises an antibody, an antigen-binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The nucleic acid can be a viral vector. The nucleic acid can be a phagemid.

In another aspect, this document features a host cell comprising a nucleic acid of any one of the preceding two paragraphs. The host cell can be a T cell, stem cell, or NK cell.

In another aspect, this document features a host cell that expresses a chimeric antigen receptor or a cell engager, wherein the chimeric antigen receptor comprises (or consists essentially of, or consists of) an antigen binding domain, a hinge, a transmembrane domain, and one or more signaling domains, wherein the antigen binding domain comprises an antibody, an antigen-binding fragment, or an antibody domain, wherein the cell engager comprises (or consists essentially of, or consists of) a first antigen binding domain, a linker, and a second antigen binding domain, wherein the first antigen binding domain comprises an antibody, an antigen-binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The host cell can be a T cell, stem cell, or NK cell.

In another aspect, this document features an antibody-drug conjugate (ADC) comprising (or consisting essentially of, or consisting of) an antigen binding domain covalently linked to a drug, wherein the antigen binding domain comprises an antibody, an antigen binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antigen binding domain can comprise a scFv having the ability to bind to a CD276 polypeptide. The antigen binding domain can comprise a VH domain having the ability to bind to a CD276 polypeptide. The drug can be selected from the group consisting of auristatins, mertansine, or pyrrolobenzodiazepine (PBD) dimers.

In another aspect, this document features a composition comprising (or consisting essentially of, or consisting of) an antibody, an antigen binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The composition can comprise the antibody of any one of (a-i)-(a-x). The composition can comprise the antigen binding fragment of any one of (b-i)-(b-x). The composition can comprise the antibody domain of any one of (c-i)-(c-vi). The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) a cell engager, wherein the cell engager comprises (or consists essentially of, or consists of) a first antigen binding domain, a linker, and a second antigen binding domain, wherein the first antigen binding domain comprises (or consists essentially of, or consists of) an antibody, an antigen-binding fragment, or an antibody domain,
wherein the antibody comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The first antigen binding domain can comprise a scFv having the ability to bind to a CD276 polypeptide. The first antigen binding domain can comprise a VH domain having the ability to bind to a CD276 polypeptide. The linker can comprise a linker set forth in FIG. 10 or FIG. 13B. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 16. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 17. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 17. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) a cell comprising a chimeric antigen receptor comprising (or consisting essentially of, or consisting of) an antigen binding domain, a hinge, a transmembrane domain, and one or more signaling domains, wherein the antigen binding domain comprises an antibody, an antigen-binding fragment, or an antibody domain,
wherein the antibody comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO: 18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antigen binding domain can comprise a scFv having the ability to bind to a CD276 polypeptide. The antigen binding domain can comprise a VH domain having the ability to bind to a CD276 polypeptide. The hinge can comprise a hinge set forth in FIG. 13B. The transmembrane domain can comprise a transmembrane domain set forth in FIG. 14. The chimeric antigen receptor can comprise one or more signaling domains set forth in FIG. 15. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) an antibody-drug conjugate (ADC) comprising (or consisting essentially of, or consisting of) an antigen binding domain covalently linked to a drug, wherein the antigen binding domain comprises an antibody, an antigen binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The antigen binding domain can comprise a scFv having the ability to bind to a CD276 polypeptide. The antigen binding domain can comprise a VH domain having the ability to bind to a CD276 polypeptide. The drug can be selected from the group consisting of auristatins, mertansine, or pyrrolobenzodiazepine (PBD) dimers. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer. The method comprises (or consists essentially of, or consists of) administering, to the mammal, a composition of any one of the preceding paragraphs. The mammal can be a human. The cancer can be a CD276⁺ cancer. The D276⁺ cancer can be selected from the group consisting of CD276⁺ liver cancer, CD276⁺ colorectal cancer, CD276⁺ brain cancer, CD276⁺ skin cancer, CD276⁺ lung cancer, CD276⁺ prostate cancer, CD276⁺ breast cancer, CD276⁺ ovarian cancer, CD276⁺ cervical cancer, CD276⁺ esophageal cancer, CD276⁺ glioma, CD276⁺ kidney cancer, CD276⁺ mesothelioma, and CD276⁺ pancreatic cancer. The number of cancer cells within the mammal can be reduced following the administering step.

In another aspect, this document features a method of treating a mammal having cancer. The method comprises (or consists essentially of, or consists of) (a) administering, to the mammal, the composition of any one of the preceding paragraphs, and (b) administering, to the mammal, a composition comprising a checkpoint inhibitor. The mammal can be a human. The cancer can be a CD276⁺ cancer. The CD276⁺ cancer can be selected from the group consisting of CD276⁺ liver cancer, CD276⁺ colorectal cancer, CD276⁺ brain cancer, CD276⁺ skin cancer, CD276⁺ lung cancer, CD276⁺ prostate cancer, CD276⁺ breast cancer, CD276⁺ ovarian cancer, CD276⁺ cervical cancer, CD276⁺ esophageal cancer, CD276⁺ glioma, CD276⁺ kidney cancer, CD276⁺ mesothelioma, and CD276⁺ pancreatic cancer. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The number of cancer cells within the mammal can be reduced following the administering steps (a) and (b).

In another aspect, this document features a method for binding a binding molecule to a CD276 polypeptide. The method comprises (or consists essentially of, or consists of) contacting the CD276 polypeptide with an antibody, an antigen binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The contacting can be performed in vitro. The contacting can be performed in vivo. The contacting can be performed within a mammal by administering the antibody, the antigen binding fragment, or the antibody domain to the mammal. The mammal can be a human.

In another aspect, this document features a method for binding a binding molecule to a CD276 polypeptide. The method comprises (or consists essentially of, or consists of) contacting the CD276 polypeptide with a chimeric antigen receptor, a cell engager, or an ADC, wherein the chimeric antigen receptor comprises (or consists essentially of, or consists of) an antigen binding domain, a hinge, a transmembrane domain, and one or more signaling domains, wherein the antigen binding domain comprises an antibody, an antigen-binding fragment, or an antibody domain, wherein the cell engager comprises (or consists essentially of, or consists of) a first antigen binding domain, a linker, and a second antigen binding domain, wherein the first antigen binding domain comprises an antibody, an antigen-binding fragment, or an antibody domain, wherein the ADC comprises (or consists essentially of, or consists of) an antigen binding domain covalently linked to a drug, wherein the antigen binding domain of the ADC comprises an antibody, an antigen-binding fragment, or an antibody domain,
wherein the antibody comprises (or consists essentially of, or consists of) (a-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (a-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (a-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (a-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (a-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (a-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (a-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (a-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (a-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (a-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions);
wherein the antigen-binding fragment comprises (or consists essentially of, or consists of) (b-i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (b-ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (b-iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (b-iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (b-v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (b-vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (b-vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (b-viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions); (b-ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (b-x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions); and
wherein the antibody domain comprises (or consists essentially of, or consists of) (c-i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (c-ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (c-iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (c-iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (c-v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or (c-vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions). The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts amino acid residues 1 to 466 of a human CD276 polypeptide (SEQ ID NO:81). The underlined amino acid sequence (residues 29 to 461) of this human CD276 polypeptide depicts an extracellular domain (SEQ ID NO:82). The bold amino acid sequence (residues 358 to 466) of this human CD276 polypeptide depicts a sequence that includes an Ig-like C2-type 2 domain (SEQ ID NO:471).

FIGS. 2A and 2B depict the amino acid sequences of the heavy chain variable domain (FIG. 2A) and the light chain variable domain (FIG. 2B) of an Fab designated Clone #1 (ab1). The CDRs and framework sequences of each also are delineated.

FIGS. 3A and 3B depict the amino acid sequences of the heavy chain variable domain (FIG. 3A) and the light chain variable domain (FIG. 3B) of an Fab designated Clone #2 (ab2). The CDRs and framework sequences of each also are delineated.

FIGS. 4A and 4B depict the amino acid sequences of the heavy chain variable domain (FIG. 4A) and the light chain variable domain (FIG. 4B) of an Fab designated Clone #3 (ab3). The CDRs and framework sequences of each also are delineated.

FIG. 5 depicts the amino acid sequence of a VH domain designated Clone #4 (ab4). The CDRs and framework sequences also are delineated.

FIG. 6 depicts the amino acid sequence of a VH domain designated Clone #5 (ab5). The CDRs and framework sequences also are delineated.

FIG. 7 depicts the amino acid sequence of a VH domain designated Clone #6 (ab6). The CDRs and framework sequences also are delineated.

FIG. 8 depicts the amino acid sequence of a VH domain designated Clone #7 (ab7). The CDRs and framework sequences also are delineated.

FIG. 9 depicts the nucleic acid sequences encoding the indicated chains/domains of Clones #1-#10.

FIG. 10 depicts exemplary linker amino acid sequences that can be used to link a heavy chain variable domain and a light chain variable domain together to form a scFv.

FIG. 11A depicts exemplary scFv structures. FIGS. 11B and 11C depict the amino acid sequences of an exemplary heavy chain variable domain (FIG. 11B) and an exemplary light chain variable domain (FIG. 11C) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIGS. 12A and 12B depict the amino acid sequences of an exemplary heavy chain variable domain (FIG. 12A) and an exemplary light chain variable domain (FIG. 12B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIG. 13A depicts the amino acid sequences of exemplary signal peptide that can be used to design a CAR. FIG. 13B depicts the amino acid sequences of exemplary hinges that can be used to design a CAR.

FIG. 14 depicts the amino acid sequences of exemplary transmembrane domains that can be used to design a CAR.

FIG. 15 depicts the amino acid sequences of exemplary intracellular signaling domains that can be used to design a CAR.

FIG. 16 depicts the amino acid sequences of exemplary antigen binding domains that can be used to design cell engagers that bind to T cells.

FIG. 17 depicts the amino acid sequences of exemplary antigen binding domains that can be used to design cell engagers that bind to NK cells.

FIGS. 18A and 18B depict the amino acid sequences of the heavy chain variable domain (FIG. 18A) and the light chain variable domain (FIG. 18B) of an Fab designated Clone #8 (ab8). The CDRs and framework sequences of each also are delineated.

FIG. 19A depicts exemplary CAR structures. FIGS. 19B and 19C depict schematics of exemplary CAR constructs designed to express a CAR. A promotor sequence (e.g., a CMV immediate early promotor sequence) can be followed by a signal peptide sequence (e.g., a GM-CSF signal peptide sequence or a signal peptide set forth in FIG. 13A), followed by a scFv or VH domain provided herein (e.g., a scFv designed to include two sets of three CDRs such as CDR1, CDR2, and CDR3 of a heavy chain and CDR1, CDR2, and CDR3 of a light chain (in either order) of an antigen binding fragment provided herein, for example, SEQ ID NOs:1-3 and 9-11; SEQ ID NOs:17-19 and 25-27; SEQ ID NOs:33-35 and 41-43; or SEQ ID NOs:473-475 and 481-483), followed by a linker (e.g., SEQ ID NO:516), followed by a CD8 hinge sequence (e.g., SEQ ID NO:436), followed by a CD8 transmembrane sequence (e.g., SEQ ID NO:514), followed by a human 4-1BB (CD137) intracellular signaling domain sequence (e.g., SEQ ID NO:446), followed by a human CD3ζ intracellular signaling domain sequence (e.g., SEQ ID NO:445). In some cases, the scFv can be replaced with a VH domain provided herein (e.g., a VH domain designed to include a single set of three CDRs of an antibody domain provided herein, for example, SEQ ID NOs:49-51; SEQ ID NOs:57-59; SEQ ID NOs:65-67; SEQ ID NOs:73-75; SEQ ID NOs:529-531; or SEQ ID NOs:537-539).

FIG. 20 depicts an amino acid of a CAR (CAR #1) designed to include a scFv created using the CDRs of the clone #1 Fab and a nucleic acid sequence encoding that CAR. The light chain variable domain (SEQ ID NO:16 without kappa constant domain) of clone #1 is followed by a GGGSGGGG linker (SEQ ID NO:517), followed by the heavy chain variable domain (SEQ ID NO:8 without CH1) of clone #1, followed by a GQAGR linker sequence (SEQ ID NO:516), followed by a CD8 hinge sequence (SEQ ID NO:436), followed by a CD8 transmembrane domain sequence (SEQ ID NO:514), followed by a human 4-1BB (CD137) intracellular signaling domain sequence (SEQ ID NO:446), followed by a human CD3ζ intracellular signaling domain sequence (SEQ ID NO:445).

FIG. 21 depicts an amino acid of a CAR (CAR #2) designed to include a scFv created using the CDRs of the clone #2 Fab and a nucleic acid sequence encoding that CAR. The light chain variable domain (SEQ ID NO:32 without kappa constant domain) of clone #2 is followed by a SGGGG linker (SEQ ID NO:518), followed by the heavy chain variable domain (SEQ ID NO:24 without CH1) of clone #2, followed by a GQAGR linker sequence (SEQ ID NO:516), followed by a CD8 hinge sequence (SEQ ID NO:436), followed by a CD8 transmembrane domain sequence (SEQ ID NO:514), followed by a human 4-1BB (CD137) intracellular signaling domain sequence (SEQ ID NO:446), followed by a human CD3ζ intracellular signaling domain sequence (SEQ ID NO:445).

FIG. 22 depicts an amino acid of a CAR (CAR #3) designed to include a scFv created using the CDRs of the clone #3 Fab and a nucleic acid sequence encoding that CAR. The heavy chain variable domain (SEQ ID NO:40 without CH1) of clone #3 is followed by a GGGGS linker sequence (SEQ ID NO:519), followed by the light chain variable domain (SEQ ID NO:48 without kappa constant domain) of clone #3, followed by a GQAGR linker sequence (SEQ ID NO:516), followed by a CD8 hinge sequence (SEQ ID NO:436), followed by a CD8 transmembrane domain sequence (SEQ ID NO:514), followed by a human 4-1BB (CD137) intracellular signaling domain sequence (SEQ ID NO:446), followed by a human CD3ζ intracellular signaling domain sequence (SEQ ID NO:445).

FIG. 23A is a graph plotting the percent lysis of target cells (CHO-K1-CD276-Luci cells) by effector cells (T cells transfected with CAR #1 of FIG. 20 or T cells transfected with CAR #3 of FIG. 23). Untransduced T cells were used as control effector cells. FIG. 23B is a graph plotting the percent lysis of target cells (CHO-K1-CD276-Luci, PC3, or Du145 cells) by effector cells (T cells transfected with CAR #2 of FIG. 21). Untransduced T cells were used as control effector cells.

FIG. 24A is a schematic of an exemplary BiTE designed using CDR1, CDR2, and CDR3 of a heavy chain provided herein and CDR1, CDR2, and CDR3 of a light chain provided herein in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (G₄S)₃ linker (SEQ ID NO: 95). FIG. 24B is a schematic of an exemplary BiTE designed using CDR1, CDR2, and CDR3 of a VH domain provided herein. A humanized anti-CD3 scFv (e.g., an gOKT3-7 scFv set forth in U.S. Pat. No. 6,750,325) can be linked to the C-terminus of the VH domain via a linker (e.g., a (G₄S)₃ linker (SEQ ID NO: 95). FIG. 24C depicts an amino acid of a linker sequence (SEQ ID NO:95) followed by an gOKT3-7 scFv sequence, which can be attached to a light chain as shown in FIG. 24A or a VH domain as shown in FIG. 24B. FIG. 24C also depicts a nucleic acid sequence encoding that linker and gOKT3-7 scFv.

FIG. 25A is a graph plotting the percent lysis of target cells (CHO-K1-CD276-Luci cells) by effector cells (T cells) in the presence of the indicated amounts of a BiTE designed to include the CDRs of Clone #1 in the IgG1/gOKT3-7 scFv format of FIG. 24A (BiTE #1), a BiTE designed to include the CDRs of Clone #2 in the IgG1/gOKT3-7 scFv format of FIG. 24A (BiTE #2), or a BiTE designed to include the CDRs of Clone #3 in the IgG1/gOKT3-7 scFv format of FIG. 24A (BiTE #3). A Fab antibody binding to another antigen and without binding to CD276 was used as an isotype control (Control BiTE). FIG. 25B is a graph plotting the percent lysis of target cells (Du145 cells) by effector cells (T cells) in the presence of the indicated amounts of BiTE #2 or Control BiTE. FIG. 25C is a graph plotting the percent lysis of target cells (PC3 cells) by effector cells (T cells) in the presence of the indicated amounts of BiTE #2 or Control BiTE. FIG. 25D is a graph plotting the percent lysis of target cells (Du145 cells) by effector cells (T cells) in the presence of the indicated amounts of a BiTE designed to include the CDRs of Clone #5 in the VH/gOKT3-7 scFv format of FIG. 24B (BiTE #5).

FIG. 26 depicts the amino acid sequence of a VH domain designated Clone #9 (ab9). The CDRs and framework sequences also are delineated.

FIG. 27 depicts the amino acid sequence of a VH domain designated Clone #10 (ab10). The CDRs and framework sequences also are delineated.

FIG. 28 is a graph plotting binding (OD₄₅₀ nm) of the indicated binders to a recombinant CD276 polypeptide that included four Ig-like extracellar domains as measure by ELISA.

FIG. 29 is a graph plotting binding (PE (YG)-A::PE (YG)-A) of the indicated binders to CD276⁺ 293 T cells or CD276⁻ (KO) 293 T cells as measure by FACS analysis.

FIG. 30 contains graphs of a dynamic light scattering evaluation of aggregation propensity of the indicated binder at day 0, day 3, and day 7. These results demonstrate that the VH domain binders (Clones #9 and #10) formed no high molecular weight species during stressed incubation, indicating that these VH binders are thermostable.

FIG. 31 contains mass spectrum characterization results of molecular weights of the indicated binders. These results demonstrate that the VH domain binders (Clones #9 and #10) have molecular weights consistent with their amino acid sequences.

FIG. 32 contains graphs plotting response (RU; Response Unit in surface plasmon resonance SPR) versus time to assess the binding kinetics of VH binders to a CD276 polypeptide. These results demonstrate that the VH binders bear low equilibrium dissociation constants (KD) to a CD276 polypeptide (e.g., single digit nM range such as a KD of 1-9 nM).

### DETAILED DESCRIPTION

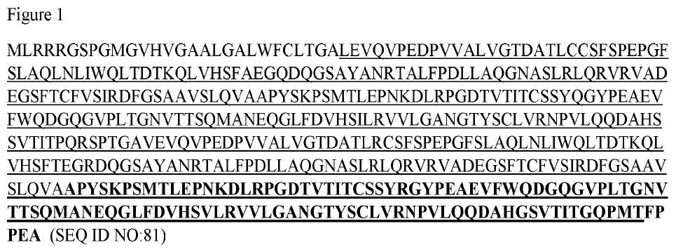

This document provides binders (e.g., antibodies, antigen binding fragments, antibody domains, CARs, cell engagers, and ADCs) that bind (e.g., specifically bind) to a CD276 polypeptide (e.g., a human CD276 polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:81 or SEQ ID NO:82 or SEQ ID NO:471 (see, e.g., FIG. 1). In some cases, a binder (e.g., an antibody, an antigen binding fragment, an antibody domain, a CAR, a cell engager, or an ADC) provided herein can have the ability to bind to a CD276 polypeptide and can lack the ability to bind to a PD-L1 polypeptide. For example, a binder (e.g., an antibody, an antigen binding fragment, an antibody domain, a CAR, a cell engager, or an ADC) provided herein can have the ability to bind to a human CD276 polypeptide and can lack the ability to bind to human PD-L1.

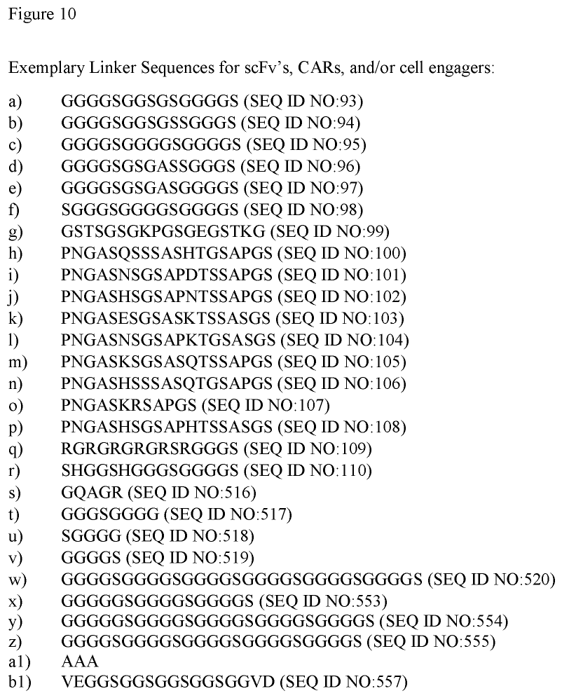

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 10). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 10). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains.

An antibody provided herein can include the CDRs as described herein (e.g., as described in Table 43) and can be configured to be a human antibody, a humanized antibody, or a chimeric antibody. In some cases, an antibody provided herein can include the CDRs as described herein (e.g., as described in Table 43) and can be a monoclonal antibody. In some cases, an antibody provided herein can include the CDRs as described herein (e.g., as described in Table 43) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 43) and can be configured to be a human antigen binding fragment, a humanized antigen binding fragment, or a chimeric antigen binding fragment. In some cases, an antigen binding fragment provided herein can include the CDRs as described herein (e.g., as described in Table 43) and can be a monoclonal antigen binding fragment. In some cases, an antigen binding fragment provided herein can include the CDRs as described herein (e.g., as described in Table 43) and can be configured as an Fab antibody.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 43) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 43) and can be a monoclonal antibody domain. In some cases, an antibody domain provided herein can include the CDRs as described herein (e.g., as described in Table 43) and can be engineered as a single VH domain or a single VL domain.

An anti-CD276 antibody, anti-CD276 antigen binding fragment, or anti-CD276 antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-CD276 antibody) can be a scFv antibody. In some cases, an antigen binding fragment provided herein (e.g., an anti-CD276 antibody fragment) can be an Fab. In some cases, an antibody provided herein (e.g., an anti-CD276 antibody) can be a fully intact antibody consisting of both VH and VL. In some cases, an antibody domain provided herein (e.g., an anti-CD276 antibody domain) can be a VH domain.

The term “chimeric antigen receptor” as used herein refers to a chimeric polypeptide that is designed to include an antigen binding domain, an optional hinge, a transmembrane domain, and one or more intracellular signaling domains. As described herein, the antigen binding domain of a CAR provided herein can be designed to bind to a CD276 polypeptide (e.g., a human CD276 polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a CD276 polypeptide (e.g., a human CD276 polypeptide). In some examples, a CAR provided herein can be designed to include an antigen binding domain that includes (a) a single set of three CDRs (e.g., a CDR1, CDR2, and CDR3) of an antibody domain (e.g., a VH domain) provided herein (e.g., SEQ ID NOs:49-51; SEQ ID NOs:57-59; SEQ ID NOs:65-67; SEQ ID NOs:73-75; SEQ ID NOs:529-531; or SEQ ID NOs:537-539) or (b) two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11; SEQ ID NOs:17-19 and 25-27; SEQ ID NOs:33-35 and 41-43; or SEQ ID NOs:473-475 and 481-483). In some cases, an antigen binding domain of a CAR targeting a CD276 polypeptide can be designed to include a VH domain described herein or a scFv antibody described herein.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as hinge to make a CAR described herein. For example, any one of the linker sequences set forth in FIG. 10 can be used as a hinge of a CAR described herein.

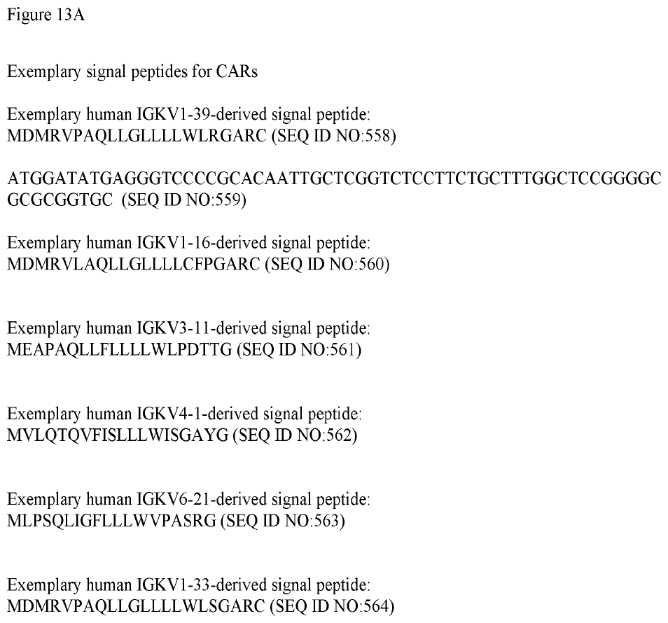

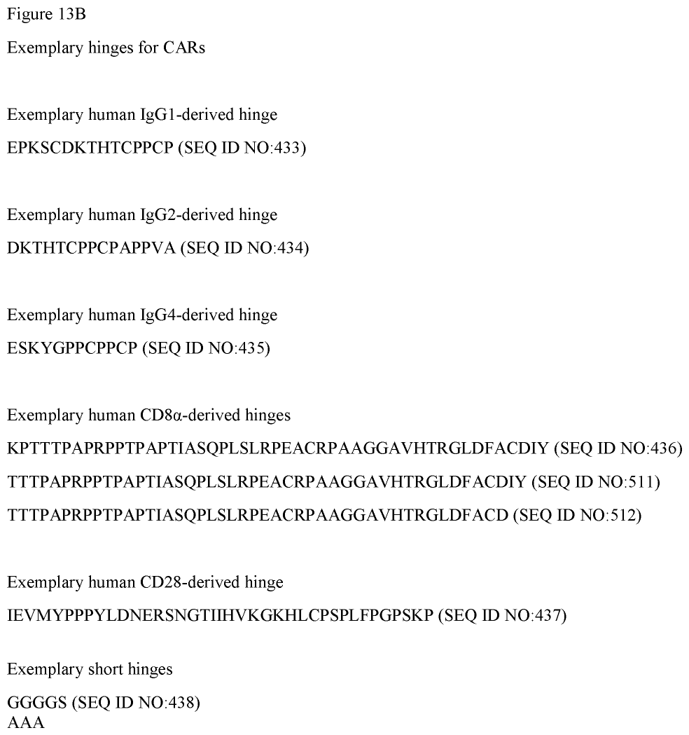

In some cases, a CAR provided herein can be designed to include a hinge that comprises, consists essentially of, or consists of one of the amino acid sequences set forth in FIG. 13B or FIG. 10. In some cases, a CAR provided herein can be designed to include a hinge that comprises, consists essentially of, or consists of one of the amino acid sequences set forth in FIG. 13B or FIG. 10 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 13B or FIG. 10 with two or less, three or less, four or less, five or less, six or less, seven or less, eight or less, nine or less, or ten or less amino acid deletions, additions, substitutions, or combinations thereof.

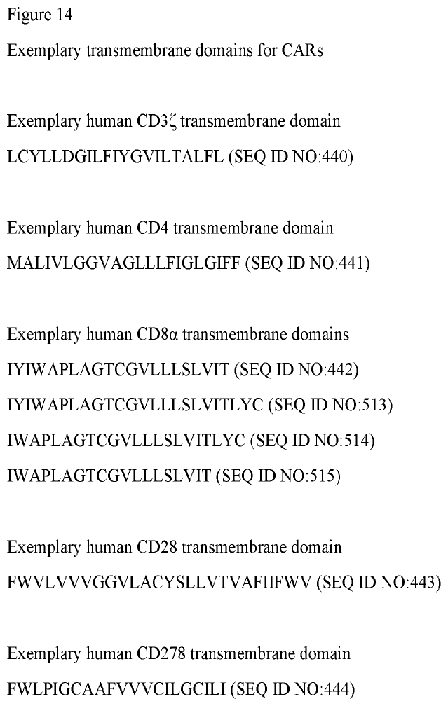

A CAR provided herein can be designed to include any appropriate transmembrane domain. For example, the transmembrane domain of a CAR provided herein can be, without limitation, a CD3ζ transmembrane domain, a CD4 transmembrane domain, a CD8α transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 14. In some cases, a CAR provided herein can be designed to include a transmembrane domain that comprises, consists essentially of, or consists of one of the amino acid sequences set forth in FIG. 14 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 14 with two or less, three or less, four or less, five or less, six or less, seven or less, eight or less, nine or less, or ten or less amino acid deletions, additions, substitutions, or combinations thereof.

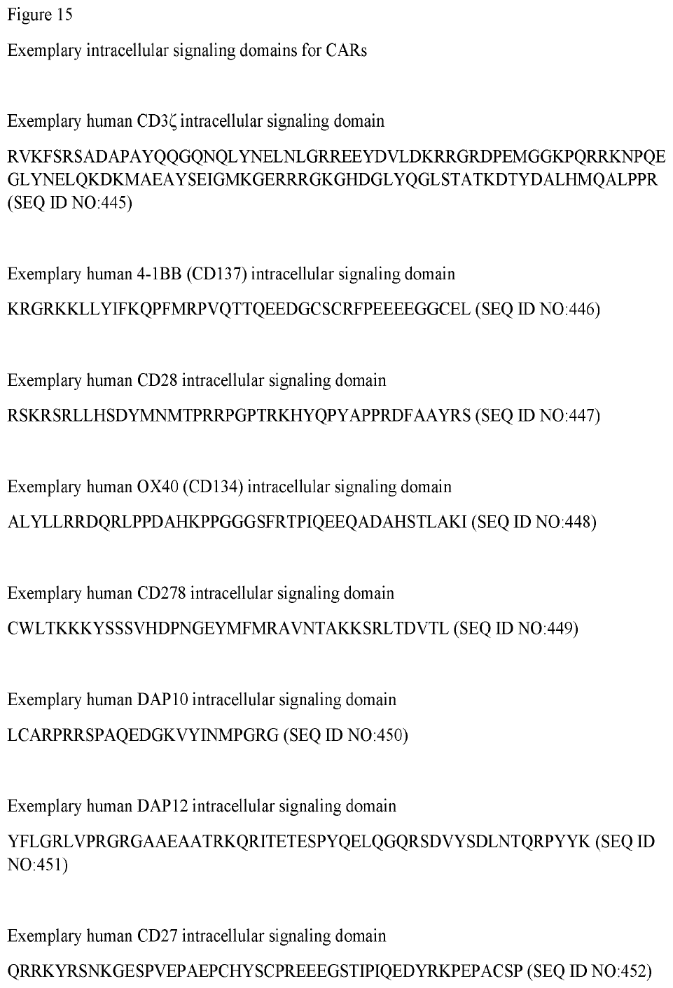

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in FIG. 15. In some cases, a CAR provided herein can be designed to include at least one intracellular signaling domain that comprises, consists essentially of, or consists of one of the amino acid sequences set forth in FIG. 15 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 15 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a CD276 polypeptide can be designed to include an scFv having a heavy chain variable domain comprising SEQ ID NO: 1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:1, SEQ ID NO:2, and SEQ ID NO:3, followed by SEQ ID NO:95, followed by a light chain variable domain comprising SEQ ID NO:9, SEQ ID NO:10, and SEQ ID NO:11, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:8, followed by SEQ ID NO:95, followed by a light chain variable domain comprising SEQ ID NO:16, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:9, SEQ ID NO:10, and SEQ ID NO:11, followed by SEQ ID NO:95, followed by a heavy chain variable domain comprising SEQ ID NO:1, SEQ ID NO:2, and SEQ ID NO:3, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:16, followed by SEQ ID NO:95, followed by a heavy chain variable domain comprising SEQ ID NO:8, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:17, SEQ ID NO:18, and SEQ ID NO:19, followed by SEQ ID NO:95, followed by a light chain variable domain comprising SEQ ID NO:25, SEQ ID NO:26, and SEQ ID NO:27, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:24, followed by SEQ ID NO:95, followed by a light chain variable domain comprising SEQ ID NO:32, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:25, SEQ ID NO:26, and SEQ ID NO:27, followed by SEQ ID NO:95, followed by a heavy chain variable domain comprising SEQ ID NO:17, SEQ ID NO:18, and SEQ ID NO: 19, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:32, followed by SEQ ID NO:95, followed by a heavy chain variable domain comprising SEQ ID NO:24, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:33, SEQ ID NO:34, and SEQ ID NO:35, followed by a linker such as a linker set forth in FIG. 10, followed by a light chain variable domain comprising SEQ ID NO:41, SEQ ID NO:42, and SEQ ID NO:43, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:33, SEQ ID NO:34, and SEQ ID NO:35, followed by SEQ ID NO:95, followed by a light chain variable domain comprising SEQ ID NO:41, SEQ ID NO:42, and SEQ ID NO:43, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:40, followed by a linker such as a linker set forth in FIG. 10, followed by a light chain variable domain comprising SEQ ID NO:48, followed by a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:40, followed by SEQ ID NO:95, followed by a light chain variable domain comprising SEQ ID NO:48, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:41, SEQ ID NO:42, and SEQ ID NO:43, followed by a linker such as a linker set forth in FIG. 10, followed by a heavy chain variable domain comprising SEQ ID NO:33, SEQ ID NO:34, and SEQ ID NO:35, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:41, SEQ ID NO:42, and SEQ ID NO:43, followed by SEQ ID NO:95, followed by a heavy chain variable domain comprising SEQ ID NO:33, SEQ ID NO:34, and SEQ ID NO:35, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:48, followed by a linker such as a linker set forth in FIG. 10, followed by a heavy chain variable domain comprising SEQ ID NO:40, followed by a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:48, followed by SEQ ID NO:95, followed by a heavy chain variable domain comprising SEQ ID NO:40, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:473, SEQ ID NO:474, and SEQ ID NO:475, followed by a linker such as a linker set forth in FIG. 10, followed by a light chain variable domain comprising SEQ ID NO:481, SEQ ID NO:482, and SEQ ID NO:483, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:473, SEQ ID NO:474, and SEQ ID NO:475, followed by SEQ ID NO:95, followed by a light chain variable domain comprising SEQ ID NO:481, SEQ ID NO:482, and SEQ ID NO:483, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a heavy chain variable domain comprising SEQ ID NO:480, followed by a linker such as a linker set forth in FIG. 10, followed by a light chain variable domain comprising SEQ ID NO:488, followed by a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a heavy chain variable domain comprising SEQ ID NO:480, followed by SEQ ID NO:95, followed by a light chain variable domain comprising SEQ ID NO:488, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:481, SEQ ID NO:482, and SEQ ID NO:483, followed by a linker such as a linker set forth in FIG. 10, followed by a heavy chain variable domain comprising SEQ ID NO:473, SEQ ID NO:474, and SEQ ID NO:475, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:481, SEQ ID NO:482, and SEQ ID NO:483, followed by SEQ ID NO:95, followed by a heavy chain variable domain comprising SEQ ID NO:473, SEQ ID NO:474, and SEQ ID NO:475, followed by SEQ ID NO:436, followed by SEQ ID NO:442, followed by SEQ ID NO:446, followed by SEQ ID NO:445.

In some cases, a CAR targeting a CD276 polypeptide can be designed to include an scFv having a light chain variable domain comprising SEQ ID NO:488, followed by a linker such as a linker set forth in FIG. 10, followed by a heavy chain variable domain comprising SEQ ID NO:480, followed by a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include an scFv having a light chain variable domain comprising SEQ ID NO:488, followed by SEQ ID NO:95, followed by a heavy chain variable domain comprising SEQ ID NO:480, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:49, SEQ ID NO:50, and SEQ ID NO:51, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:49, SEQ ID NO:50, and SEQ ID NO:51, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:56, followed by a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:56, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:57, SEQ ID NO:58, and SEQ ID NO:59, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:57, SEQ ID NO:58, and SEQ ID NO:59, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:64, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:64, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:65, SEQ ID NO:66, and SEQ ID NO:67, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:65, SEQ ID NO:66, and SEQ ID NO:67, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:72, followed by a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:72, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:73, SEQ ID NO:74, and SEQ ID NO:75, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:73, SEQ ID NO:74, and SEQ ID NO:75, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:80, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:80, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:529, SEQ ID NO:530, and SEQ ID NO:531, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:529, SEQ ID NO:530, and SEQ ID NO:531, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:536, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:536, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:537, SEQ ID NO:538, and SEQ ID NO:539, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:537, SEQ ID NO:538, and SEQ ID NO:539, followed by SEQ ID NO:436, followed by SEQ ID NO:442, followed by SEQ ID NO:446, followed by SEQ ID NO:445.

In some cases, a CAR targeting a CD276 polypeptide can be designed to include a VH domain comprising SEQ ID NO:544, followed by a hinge such as a hinge/linker set forth in FIG. 13B or FIG. 10 (e.g., a human CD8α hinge), followed by a transmembrane domain such as a transmembrane domain set forth in FIG. 14 (e.g., a human CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD276 polypeptide can be designed to include a VH domain comprising SEQ ID NO:544, followed by SEQ ID NO:436, followed by SEQ ID NO:442, followed by SEQ ID NO:446, followed by SEQ ID NO:445.

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a CD276 polypeptide (e.g., a human CD276 polypeptide) and at least one antigen binding domain having the ability to bind to an antigen expressed on the surface of a cell (e.g., a T cell or an NK cell). In some cases, a cell engager described herein can link a CD276⁺ cell (e.g., a CD276⁺ cancer cell) to another cell (e.g., a T cell or an NK cell) via the two or more antigen binding domains of the cell engager.

When a cell engager includes an antigen binding domain having the ability to bind to a CD276 polypeptide (e.g., a human CD276 polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a CD276 polypeptide (e.g., a human CD276 polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a CD276 polypeptide (e.g., a human CD276 polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a CD276 polypeptide (e.g., a human CD276 polypeptide). In some examples, a cell engager provided herein can be designed to include an antigen binding domain that includes (a) a single set of three CDRs (e.g., a CDR1, CDR2, and CDR3) of an antibody domain (e.g., a VH domain) provided herein (e.g., SEQ ID NOs:49-51; SEQ ID NOs:57-59; SEQ ID NOs:65-67; SEQ ID NOs:73-75; SEQ ID NOs:529-531; or SEQ ID NOs:537-539) or (b) two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11; SEQ ID NOs:17-19 and 25-27; SEQ ID NOs:33-35 and 41-43; or SEQ ID NOs:473-475 and 481-483). In some cases, an antigen binding domain of a cell engager targeting a CD276 polypeptide can be designed to include a VH domain described herein or a scFv/Fab antibody described herein. In some cases, an antigen binding domain of a CAR described herein that has the ability to bind to a CD276 polypeptide (e.g., a human CD276 polypeptide) can be used as an antigen binding domain of a cell engager that targets CD276⁺ cells.

As described herein, a cell engager can be designed to include at least one antigen binding domain having the ability to bind to a CD276 polypeptide (e.g., a human CD276 polypeptide) and at least one other antigen binding domain. That at least one other antigen binding domain can have the ability to bind to any appropriate antigen expressed on the surface of a cell. For example, when designing a cell engager such as a BiTE to link a CD276⁺ cell and a T cell, the cell engager can include an antigen binding domain having the ability to bind to a CD276 polypeptide (e.g., a human CD276 polypeptide) and an antigen binding domain having the ability to bind to a polypeptide expressed on the surface of a T cell. Examples example of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

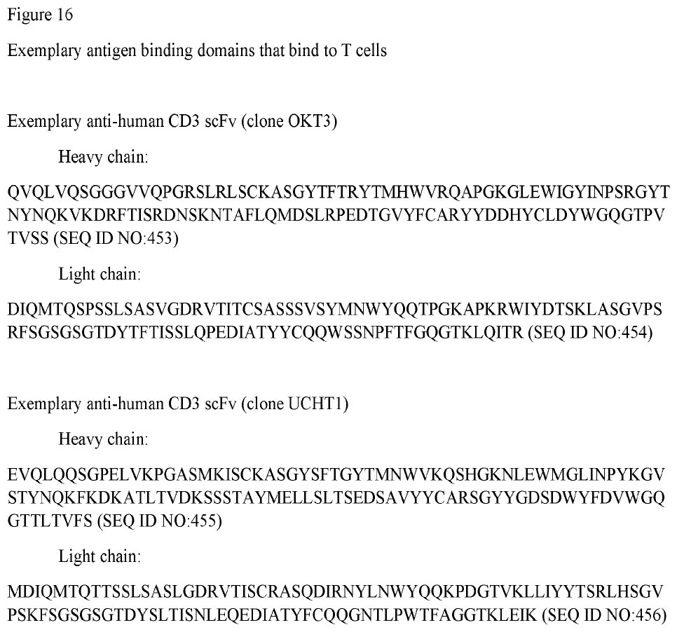

In some cases, a cell engager provided herein can be designed to include an antigen binding domain that comprises, consists essentially of, or consists of one of the amino acid sequences set forth in FIG. 16. In some cases, a cell engager provided herein can be designed to include an antigen binding domain that comprises, consists essentially of, or consists of one of the amino acid sequences set forth in FIG. 16 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 16 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link a CD276⁺ cell and an NK cell, the cell engager can include an antigen binding domain having the ability to bind to a CD276 polypeptide (e.g., a human CD276 polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

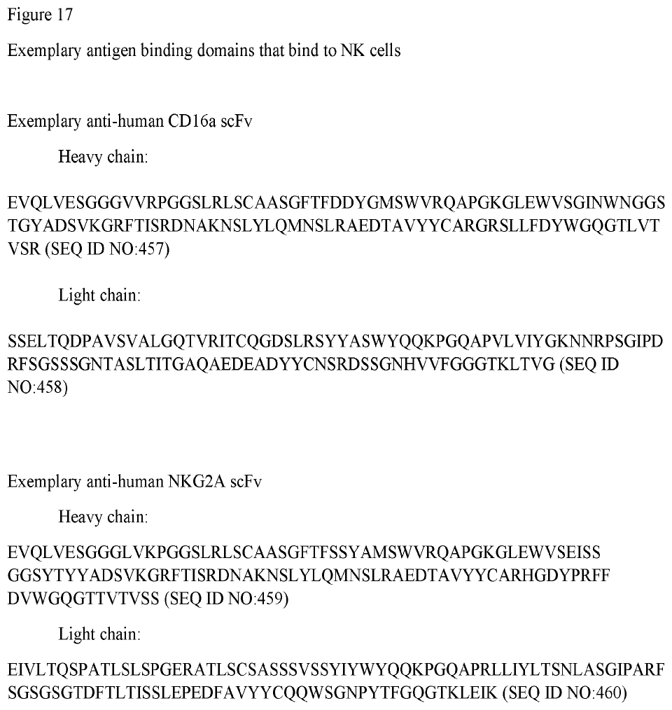

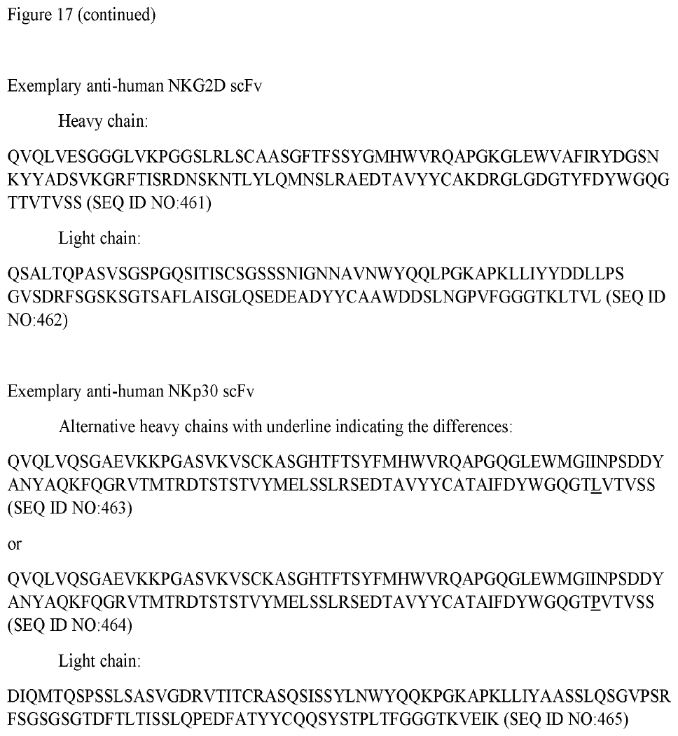

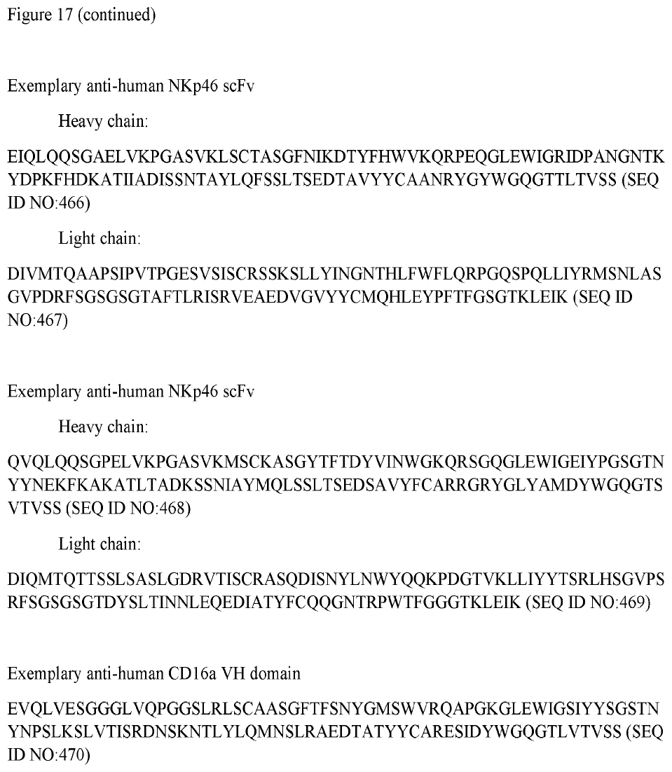

In some cases, a cell engager provided herein can be designed to include an antigen binding domain (e.g., a scFv or VH) that comprises, consists essentially of, or consists of one or more of the amino acid sequences set forth in FIG. 17. In some cases, a cell engager provided herein can be designed to include an antigen binding domain (e.g., a scFv or VH) that comprises, consists essentially of, or consists of one of the amino acid sequences set forth in FIG. 17 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 17 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 10. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:95) or a GGGGSGGGSGGGGS (SEQ ID NO:472) linker. In some cases, a hinge of a CAR described herein can be used as a linker to make a cell engager described herein. For example, any one of the sequences set forth in FIG. 13B can be used as a linker of a cell engager described herein.

In some cases, a cell engager provided herein can be designed to include a linker that comprises, consists essentially of, or consists of one of the amino acid sequences set forth in FIG. 10 or FIG. 13B. In some cases, a cell engager provided herein can be designed to include a linker that comprises, consists essentially of, or consists of one of the amino acid sequences set forth in FIG. 10 or FIG. 13B with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 10 or FIG. 13B with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a CD276 polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include an scFv having a heavy chain variable domain comprising SEQ ID NO:33, SEQ ID NO:34, and SEQ ID NO:35, followed by a linker such as a linker set forth in FIG. 10, followed by a light chain variable domain comprising SEQ ID NO:41, SEQ ID NO:42, and SEQ ID NO:43, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:41, SEQ ID NO:42, and SEQ ID NO:43, followed by a linker such as a linker set forth in FIG. 10, followed by a heavy chain variable domain comprising SEQ ID NO:33, SEQ ID NO:34, and SEQ ID NO:35, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a heavy chain variable domain comprising SEQ ID NO:40, followed by a linker such as a linker set forth in FIG. 10, followed by a light chain variable domain comprising SEQ ID NO:48, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:48, followed by a linker such as a linker set forth in FIG. 10, followed by a heavy chain variable domain comprising SEQ ID NO:40, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include an scFv having a heavy chain variable domain comprising SEQ ID NO:33, SEQ ID NO:34, and SEQ ID NO:35, followed by a linker such as a linker set forth in FIG. 10, followed by a light chain variable domain comprising SEQ ID NO:41, SEQ ID NO:42, and SEQ ID NO:43, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:41, SEQ ID NO:42, and SEQ ID NO:43, followed by a linker such as a linker set forth in FIG. 10, followed by a heavy chain variable domain comprising SEQ ID NO:33, SEQ ID NO:34, and SEQ ID NO:35, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a heavy chain variable domain comprising SEQ ID NO:40, followed by a linker such as a linker set forth in FIG. 10, followed by a light chain variable domain comprising SEQ ID NO:48, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:48, followed by a linker such as a linker set forth in FIG. 10, followed by a heavy chain variable domain comprising SEQ ID NO:40, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include an scFv having a heavy chain variable domain comprising SEQ ID NO:473, SEQ ID NO:474, and SEQ ID NO:475, followed by a linker such as a linker set forth in FIG. 10, followed by a light chain variable domain comprising SEQ ID NO:481, SEQ ID NO:482, and SEQ ID NO:483, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:481, SEQ ID NO:482, and SEQ ID NO:483, followed by a linker such as a linker set forth in FIG. 10, followed by a heavy chain variable domain comprising SEQ ID NO:473, SEQ ID NO:474, and SEQ ID NO:475, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a heavy chain variable domain comprising SEQ ID NO:480, followed by a linker such as a linker set forth in FIG. 10, followed by a light chain variable domain comprising SEQ ID NO:488, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include an scFv having a light chain variable domain comprising SEQ ID NO:488, followed by a linker such as a linker set forth in FIG. 10, followed by a heavy chain variable domain comprising SEQ ID NO:480, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include an scFv having a heavy chain variable domain comprising SEQ ID NO:473, SEQ ID NO:474, and SEQ ID NO:475, followed by a linker such as a linker set forth in FIG. 10, followed by a light chain variable domain comprising SEQ ID NO:481, SEQ ID NO:482, and SEQ ID NO:483, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:481, SEQ ID NO:482, and SEQ ID NO:483, followed by a linker such as a linker set forth in FIG. 10, followed by a heavy chain variable domain comprising SEQ ID NO:473, SEQ ID NO:474, and SEQ ID NO:475, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a heavy chain variable domain comprising SEQ ID NO:480, followed by a linker such as a linker set forth in FIG. 10, followed by a light chain variable domain comprising SEQ ID NO:488, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include an scFv having a light chain variable domain comprising SEQ ID NO:488, followed by a linker such as a linker set forth in FIG. 10, followed by a heavy chain variable domain comprising SEQ ID NO:480, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include a VH domain comprising SEQ ID NO:49, SEQ ID NO:50, and SEQ ID NO:51, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include a VH domain comprising SEQ ID NO:56, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include a VH domain comprising SEQ ID NO:49, SEQ ID NO:50, and SEQ ID NO:51, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include a VH domain comprising SEQ ID NO:56, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include a VH domain comprising SEQ ID NO:57, SEQ ID NO:58, and SEQ ID NO:59, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include a VH domain comprising SEQ ID NO:64, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include a VH domain comprising SEQ ID NO:57, SEQ ID NO:58, and SEQ ID NO:59, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include a VH domain comprising SEQ ID NO:64, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include a VH domain comprising SEQ ID NO:65, SEQ ID NO:66, and SEQ ID NO:67, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include a VH domain comprising SEQ ID NO:72, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include a VH domain comprising SEQ ID NO:65, SEQ ID NO:66, and SEQ ID NO:67, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include a VH domain comprising SEQ ID NO:72, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include a VH domain comprising SEQ ID NO:73, SEQ ID NO:74, and SEQ ID NO:75, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include a VH domain comprising SEQ ID NO:80, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include a VH domain comprising SEQ ID NO:73, SEQ ID NO:74, and SEQ ID NO:75, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include a VH domain comprising SEQ ID NO:80, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include a VH domain comprising SEQ ID NO:529, SEQ ID NO:530, and SEQ ID NO:531, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include a VH domain comprising SEQ ID NO:536, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include a VH domain comprising SEQ ID NO:529, SEQ ID NO:530, and SEQ ID NO:531, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include a VH domain comprising SEQ ID NO:536, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD276 polypeptide can be designed to include a VH domain comprising SEQ ID NO:537, SEQ ID NO:538, and SEQ ID NO:539, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiTE) targeting a CD276 polypeptide can be designed to include a VH domain comprising SEQ ID NO:544, followed by a linker such as a hinge/linker set forth in FIG. 10 or FIG. 13B (e.g., SEQ ID NO:95), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD276 polypeptide can be designed to include a VH domain comprising SEQ ID NO:537, SEQ ID NO:538, and SEQ ID NO:539, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD276 polypeptide can be designed to include a VH domain comprising SEQ ID NO:544, followed by a linker such as a hinge/linker set forth in FIG. 10 or FIG. 13B (e.g., SEQ ID NO:95), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

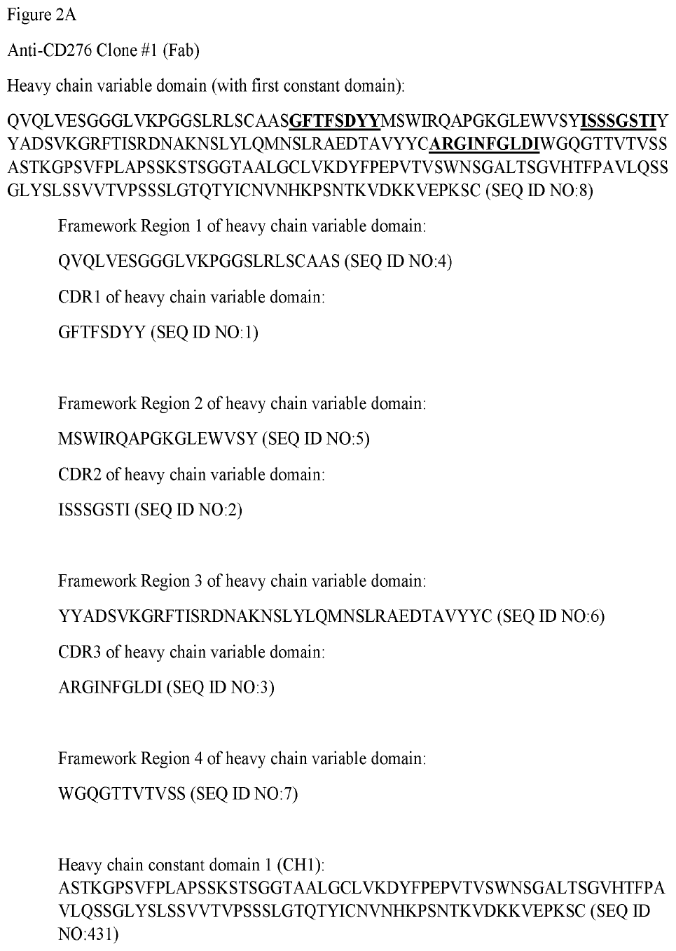

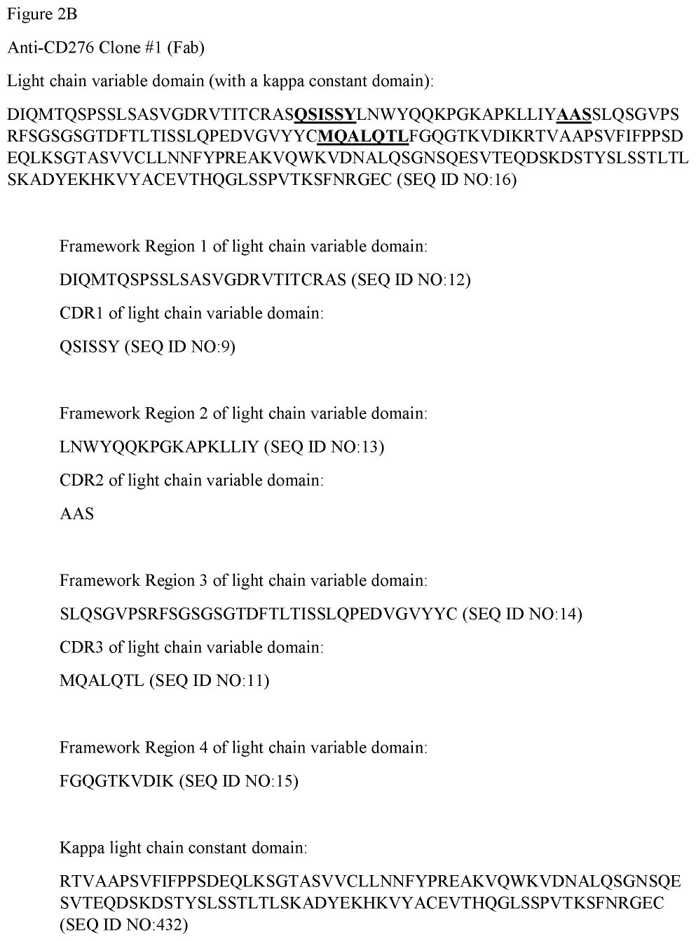

In one embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (i) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications). An example of such an antigen binding fragment having these CDRs and the ability to bind to a CD276 polypeptide (e.g., a human CD276 polypeptide) includes, without limitation, the Fab set forth in FIGS. 2A and 2B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

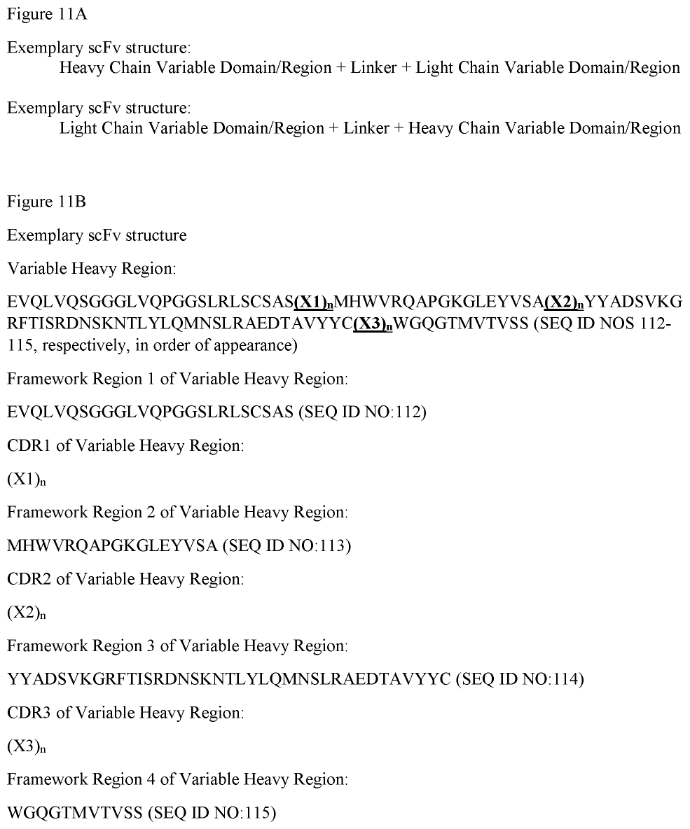

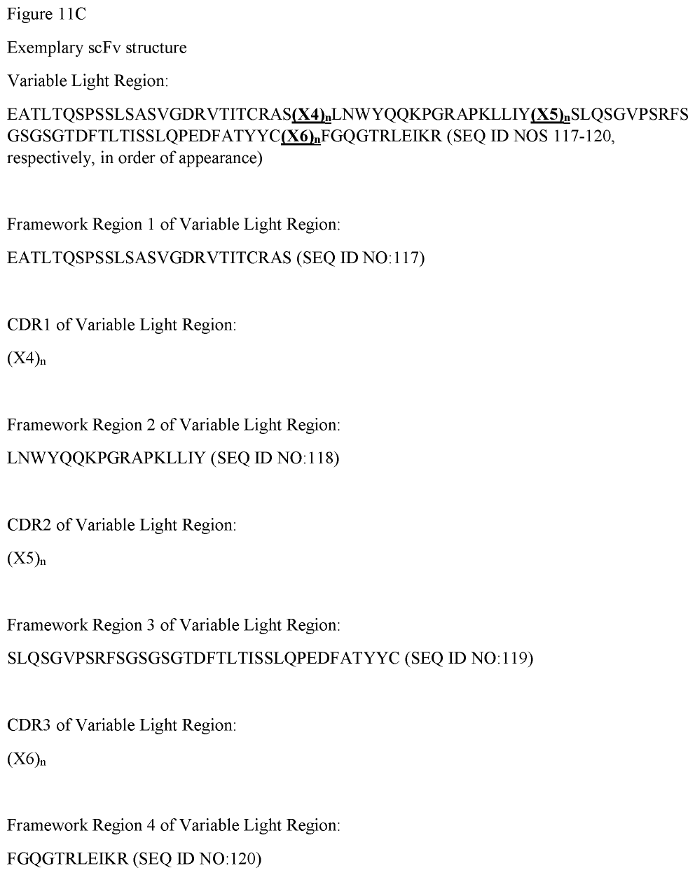

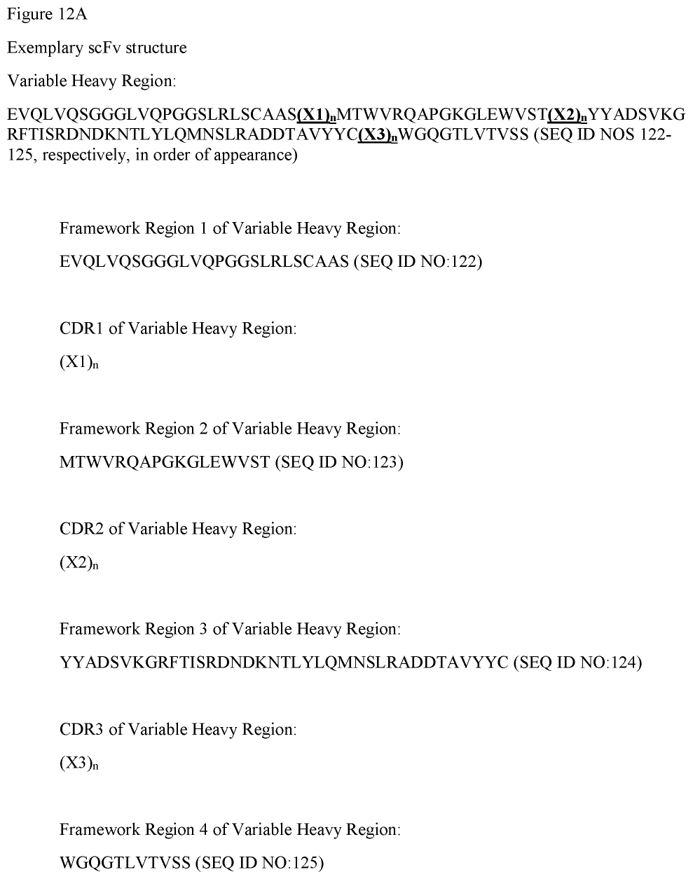

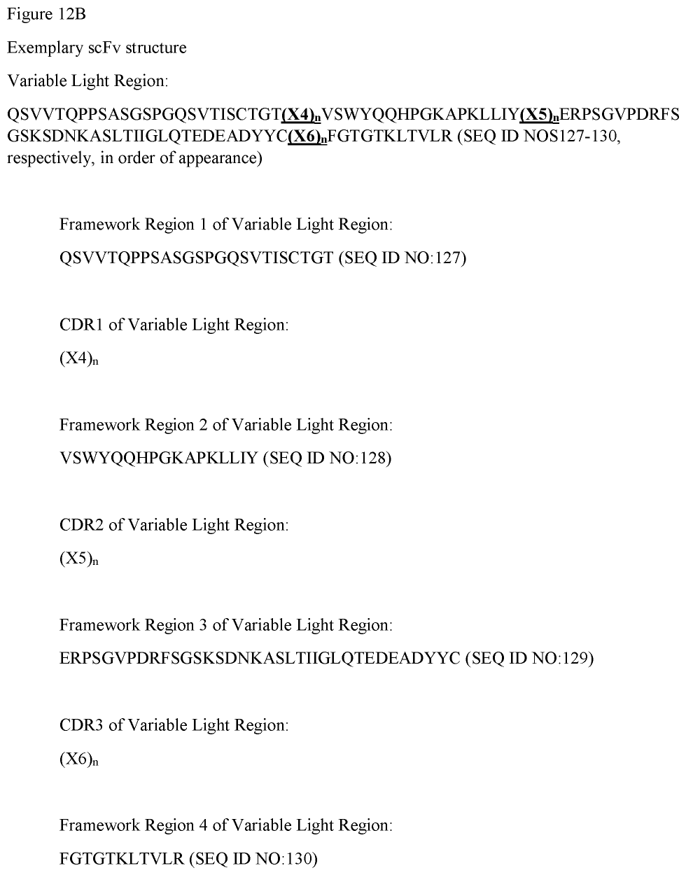

In some cases, a binder (e.g., an antibody, antigen binding fragment, antibody domain, a CAR, a cell engager, and/or an ADC) having any of the CDRs set forth in FIG. 2A or 2B can be designed to include framework regions as set forth in FIG. 2A and 2B or can be designed to include one or more framework regions from another antibody, antibody fragment, or antibody domain. For example, an Fab can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:20 or a framework region 1 having the amino acid set forth in SEQ ID NO:36. In another example, an Fab can be designed to include the six CDRs set forth in FIGS. 2A and 2B and the framework regions set forth in FIGS. 2A and 2B except that framework region 1 having the amino acid set forth in SEQ ID NO:12 is replaced with a framework region 1 having the amino acid set forth in SEQ ID NO:28 or a framework region 1 having the amino acid set forth in SEQ ID NO:44. In another example, a scFv can be designed to include the six CDRs set forth in FIGS. 2A and 2B and the framework regions set forth in FIGS. 11A and 11B or the framework regions set forth in FIGS. 12A and 12B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO:16, provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD276 polypeptide (e.g., a human CD276 polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3, and can include a light chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| GFTFDDYA | 131 |
| GFTFSSYD | 132 |
| GYTFTGYY | 133 |
| GFTFSSYW | 134 |
| GFTFSGSA | 135 |
| GFTFSSYA | 136 |
| GFTFGDYA | 137 |
| GFTVSSNE | 138 |
| GFTFSSYG | 139 |
| GFTFSYYY | 140 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| IGTAGDT | 141 |
| IKQDGSEK | 142 |
| IKSKTDGGTT | 143 |
| ISGSGGST | 144 |
| ISYDGSNK | 145 |
| ISGGST | 146 |
| ISSSSSTI | 147 |
| ISWNSGSI | 148 |
| IKQDGSEK | 149 |
| ISAYNGNT | 150 |

As used herein, a “CDR3 that consists essentially of the amino acid sequence set forth in SEQ ID NO:3” is a CDR3 that has zero, one, or two amino acid substitutions within SEQ TD NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:3 include, without limitation, those set forth in Table 3.

TABLE 3
Exemplary CDR3s that consist essentially of the
amino acid sequence set forth in SEQ ID NO: 3.

| Sequence | SEQ ID NO: |
|---|---|
| ATGINFGLSI | 151 |
| AAGINFGLDI | 152 |
| VRGINFGLYI | 153 |
| AHRGINFGLDI | 154 |
| ARRGINFGLYI | 155 |
| ARIGINFGLDI | 156 |
| AKDGINFGLDI | 157 |
| TTGINFGLYI | 158 |
| SRGINFGLDI | 159 |
| AKGINFGLSI | 160 |

As used herein, a “CDR1 that consists essentially of the amino acid sequence set forth in SEQ ID NO:9” is a CDR1 that has zero, one, or two amino acid substitutions within SEQ TD NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:9 include, without limitation, those set forth in Table 4.

TABLE 4
Exemplary CDR1s that consist essentially of the
amino acid sequence set forth in SEQ ID NO: 9.

| Sequence | SEQ ID NO: |
|---|---|
| QSISSW | 161 |
| QGISSY | 162 |
| QGISNY | 163 |
| QGISSW | 164 |
| QGISSA | 165 |
| QSVSSY | 166 |
| QSVSSN | 167 |
| QSVSSSY | 168 |
| QSIGSS | 169 |
| QSVSSSY | 170 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:10 include, without limitation, those set forth in Table 5.

TABLE 5
Exemplary CDR2s that consist essentially of the
amino acid sequence set forth in SEQ ID NO: 10.

Sequence
DAS
SAS
DAK
YAS
TIS
RVS
KVS
LGS
EVS
EAT

As used herein, a “CDR3 that consists essentially of the amino acid sequence set forth in SEQ ID NO:11” is a CDR3 that has zero, one, or two amino acid substitutions within SEQ ID NO:11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:11 include, without limitation, those set forth in Table 6.

TABLE 6
Exemplary CDR3s that consist essentially of the
amino acid sequence set forth in SEQ ID NO: 11.

| Sequence | SEQ ID NO: |
|---|---|
| MQALQTL | 181 |
| QQALQTL | 182 |
| LQALQTL | 183 |
| QKALQTL | 184 |
| MQALQSL | 185 |
| MQALPTL | 186 |
| HQALQTL | 187 |
| TQALQTL | 188 |
| EQALQTL | 189 |
| KQALQTL | 190 |

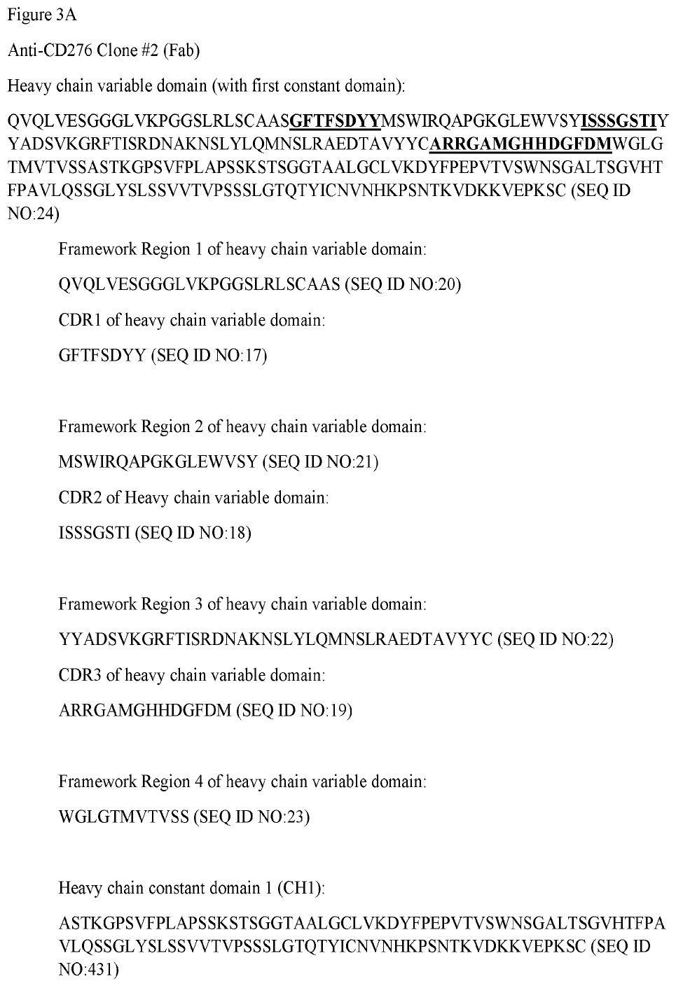

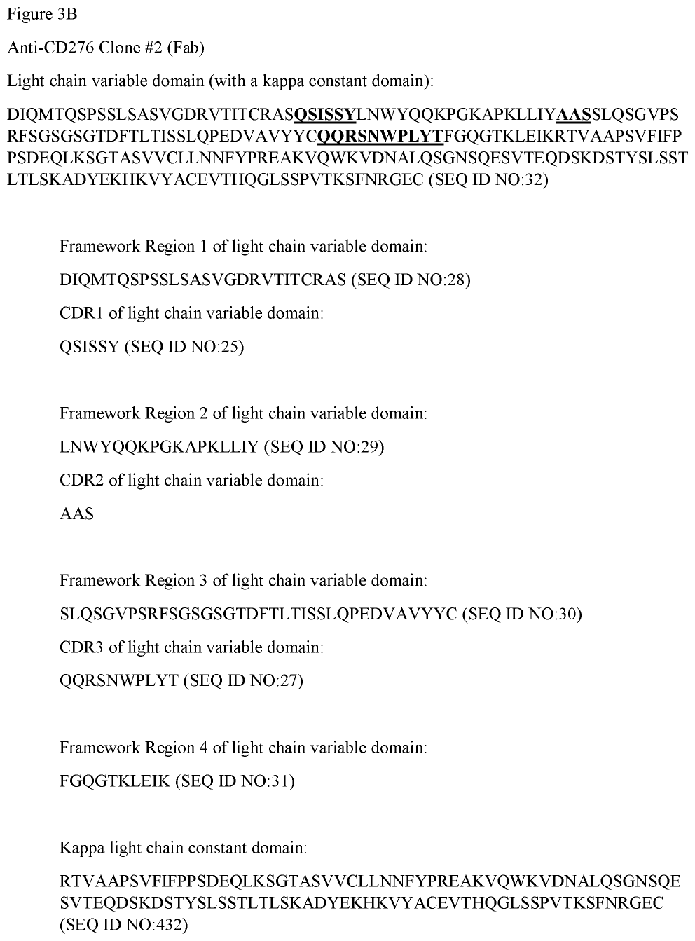

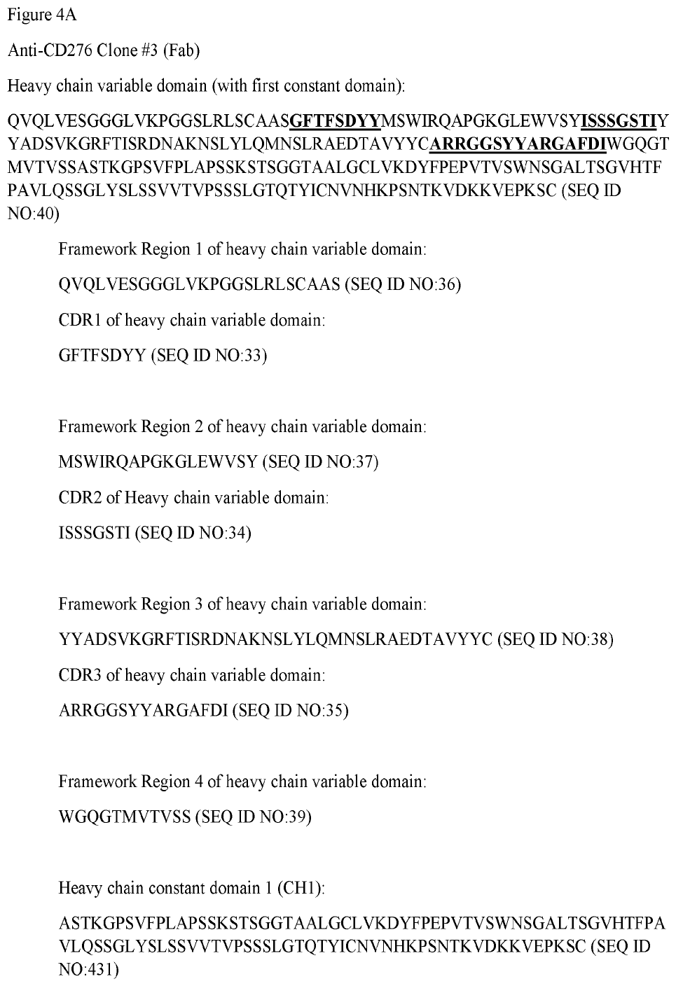

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (i) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications); and/or (ii) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications). An example of such an antigen binding fragment having these CDRs and the ability to bind to a CD276 polypeptide (e.g., a human CD276 polypeptide) includes, without limitation, the Fab set forth in FIGS. 3A and 3B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having (a) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) and/or (b) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications) and/or (b) a light chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 3A or 3B can be designed to include framework regions as set forth in FIGS. 3A and 3B or can be designed to include one or more framework regions from another antibody or antibody fragment. For example, an Fab can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:4 or a framework region 1 having the amino acid set forth in SEQ ID NO:36. In another example, an Fab can be designed to include the six CDRs set forth in FIGS. 3A and 3B and the framework regions set forth in FIGS. 3A and 3B except that framework region 1 having the amino acid set forth in SEQ ID NO:28 is replaced with a framework region 1 having the amino acid set forth in SEQ ID NO:12 or a framework region 1 having the amino acid set forth in SEQ ID NO:44. In another example, a scFv can be designed to include the six CDRs set forth in FIGS. 3A and 3B and the framework regions set forth in FIGS. 11A and 11B or the framework regions set forth in FIGS. 12A and 12B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO:32, provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD276 polypeptide (e.g., a human CD276 polypeptide), can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:17 include, without limitation, those set forth in Table 7.

TABLE 7
Exemplary CDR1s that consist essentially of the
amino acid sequence set forth in SEQ ID NO: 17.

| Sequence | SEQ ID NO: |
|---|---|
| GFTFDDYA | 191 |
| GFTFSSYD | 192 |
| GYTFTGYY | 193 |
| GFTFSSYW | 194 |
| GFTFSGSA | 195 |
| GFTFSSYA | 196 |
| GFTFGDYA | 197 |
| GFTVSSNE | 198 |
| GFTFSSYG | 199 |
| GFTFSYYY | 200 |

As used herein, a “CDR2 that consists essentially of the amino acid sequence set forth in SEQ ID NO: 18” is a CDR2 that has zero, one, or two amino acid substitutions within SEQ TD NO: 18, that has zero, one, two, three, four, or five amino acid residues directly preceding SEQ ID NO:18, and/or that has zero, one, two, three, four, or five amino acid residues directly following SEQ ID NO: 18, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO: 18 include, without limitation, those set forth in Table 8.

TABLE 8
Exemplary CDR2s that consist essentially of the
amino acid sequence set forth in SEQ ID NO: 18.

| Sequence | SEQ ID NO: |
|---|---|
| IGTAGDT | 201 |
| IKQDGSEK | 202 |
| IKSKTDGGTT | 203 |
| ISGSGGST | 204 |
| ISYDGSNK | 205 |
| ISGGST | 206 |
| ISSSSSTI | 207 |
| ISWNSGSI | 208 |
| IKQDGSEK | 209 |
| ISAYNGNT | 210 |

As used herein, a “CDR3 that consists essentially of the amino acid sequence set forth in SEQ ID NO:19” is a CDR3 that has zero, one, or two amino acid substitutions within SEQ ID NO:19, that has zero, one, two, three, four, or five amino acid residues directly preceding SEQ ID NO:19, and/or that has zero, one, two, three, four, or five amino acid residues directly following SEQ ID NO:19, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:19 include, without limitation, those set forth in Table 9.

TABLE 9
Exemplary CDR3s that consist essentially of the
amino acid sequence set forth in SEQ ID NO: 19.

| Sequence | SEQ ID NO: |
|---|---|
| YRRGAMGHHDGFDM | 211 |
| ARRGAMGHHDGFSM | 212 |
| ARRGYMGHHDGFDM | 213 |
| ARRGAMGHHSGFDM | 214 |
| SRRGAMGHHDGFDM | 215 |
| VRRGAMGHHDGFDM | 216 |
| AKRGAMGHHDGFDM | 217 |
| AGRGAMGHHDGFDM | 218 |
| ARIRGAMGHHDGFDM | 219 |
| AHRRGAMGHHDGFDM | 220 |

As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:25 include, without limitation, those set forth in Table 10.

TABLE 10
Exemplary CDR1s that consist essentially of the
amino acid sequence set forth in SEQ ID NO: 25.

| Sequence | SEQ ID NO: |
|---|---|
| QSISSW | 221 |
| QGISSY | 222 |
| QGISNY | 223 |
| QGISSW | 224 |
| QGISSA | 225 |
| QSVSSY | 226 |
| QSVSSN | 227 |
| QSVSSSY | 228 |
| QSIGSS | 229 |
| QSVSSSY | 230 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:26 include, without limitation, those set forth in Table 11.

TABLE 11
Exemplary CDR2s that consist essentially of the
amino acid sequence set forth in SEQ ID NO: 26.

Sequence
DAS
SAS
DAK
YAS
TIS
RVS
KVS
LGS
EVS
EAT

As used herein, a “CDR3 that consists essentially of the amino acid sequence set forth in SEQ ID NO:27” is a CDR3 that has zero, one, or two amino acid substitutions within SEQ TD NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:27 include, without limitation, those set forth in Table 12.

TABLE 12
Exemplary CDR3s that consist essentially of the
amino acid sequence set forth in SEQ ID NO: 27.

| Sequence | SEQ ID NO: |
|---|---|
| MQRSNWPLYT | 241 |
| HQRSNWPLYT | 242 |
| LQRSNWPLYT | 243 |
| QQRTNWPLYT | 244 |
| QQRSNWPLYS | 245 |
| TQRSNWPLYT | 246 |
| EQRSNWPLYT | 247 |
| KQRSNWPLYT | 248 |
| QRSNWPLYT | 249 |
| QQRANWPLYT | 250 |

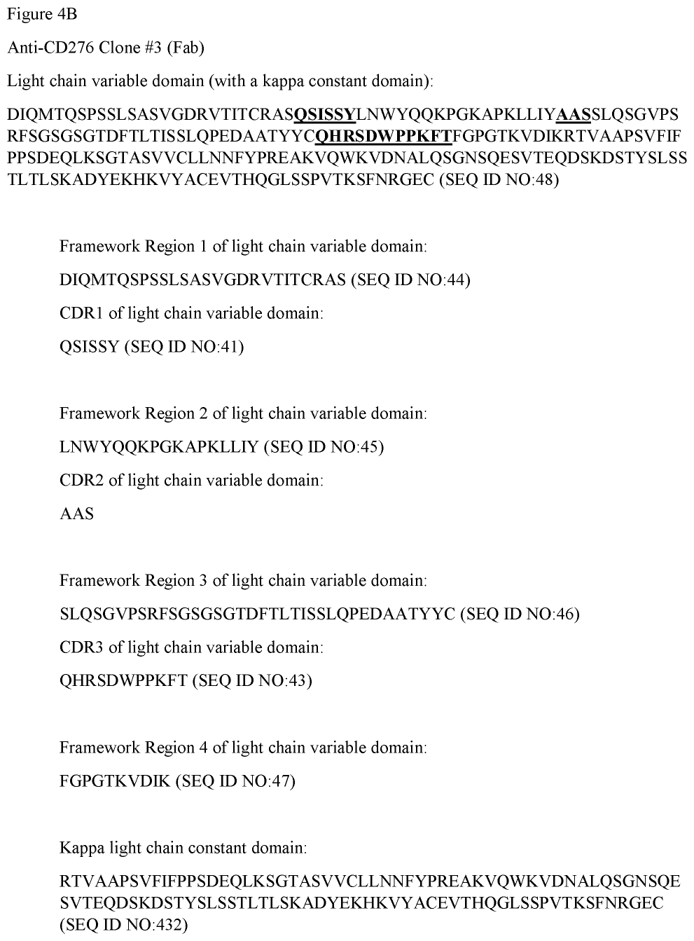

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (i) a heavy chain variable domain having a CDR1 having the amino acid sequence set forth in SEQ ID NO:33 (or a variant of SEQ ID NO:33 with one or two amino acid modifications), a CDR2 having the amino acid sequence set forth in SEQ ID NO:34 (or a variant of SEQ ID NO:34 with one or two amino acid modifications), and a CDR3 having the amino acid sequence set forth in SEQ ID NO:35 (or a variant of SEQ ID NO:35 with one or two amino acid modifications); and/or (ii) a light chain variable domain having a CDR1 having the amino acid sequence set forth in SEQ ID NO:41 (or a variant of SEQ ID NO:41 with one or two amino acid modifications), a CDR2 having the amino acid sequence set forth in SEQ ID NO:42 (or a variant of SEQ ID NO:42 with one or two amino acid modifications), and a CDR3 having the amino acid sequence set forth SEQ ID NO:43 (or a variant of SEQ ID NO:43 with one or two amino acid modifications). An example of such an antibody having these CDRs and the ability to bind to a CD276 polypeptide (e.g., a human CD276 polypeptide) includes, without limitation, the Fab set forth in FIGS. 4A and 4B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having (a) a heavy chain variable domain having a CDR1 having the amino acid sequence set forth in SEQ ID NO:33 (or a variant of SEQ ID NO:33 with one or two amino acid modifications), a CDR2 having the amino acid sequence set forth in SEQ ID NO:34 (or a variant of SEQ ID NO:34 with one or two amino acid modifications), and a CDR3 having the amino acid sequence set forth in SEQ ID NO:35 (or a variant of SEQ ID NO:35 with one or two amino acid modifications) and/or (b) a light chain variable domain having a CDR1 having the amino acid sequence set forth in SEQ ID NO:41 (or a variant of SEQ ID NO:41 with one or two amino acid modifications), a CDR2 having the amino acid sequence set forth in SEQ ID NO:42 (or a variant of SEQ ID NO:42 with one or two amino acid modifications), and a CDR3 having the amino acid sequence set forth SEQ ID NO:43 (or a variant of SEQ ID NO:43 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:36 (or a variant of SEQ ID NO:36 with one, two, three, four, five, six, seven, eight, nine, ten, or more amino acid modifications), a framework region 2 having the amino acid sequence set forth in SEQ ID NO:37 (or a variant of SEQ ID NO:37 with one, two, three, four, five, six, seven, eight, nine, ten, or more amino acid modifications), a framework region 3 having the amino acid sequence set forth in SEQ ID NO:38 (or a variant of SEQ ID NO:38 with one, two, three, four, five, six, seven, eight, nine, ten, or more amino acid modifications), and a framework region 4 having the amino acid sequence set forth in SEQ ID NO:39 (or a variant of SEQ ID NO:39 with one, two, three, four, five, six, seven, eight, nine, ten, or more amino acid modifications) and/or (b) a light chain variable domain that includes a framework region 1 having the amino acid sequence set forth in SEQ ID NO:44 (or a variant of SEQ ID NO:44 with one, two, three, four, five, six, seven, eight, nine, ten, or more amino acid modifications), a framework region 2 having the amino acid sequence set forth in SEQ ID NO:45 (or a variant of SEQ ID NO:45 with one, two, three, four, five, six, seven, eight, nine, ten, or more amino acid modifications), a framework region 3 having the amino acid sequence set forth in SEQ ID NO:46 (or a variant of SEQ ID NO:46 with one, two, three, four, five, six, seven, eight, nine, ten, or more amino acid modifications), and a framework region 4 having the amino acid sequence set forth in SEQ ID NO:47 (or a variant of SEQ ID NO:47 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 4A or 4B can be designed to include framework regions as set forth in FIGS. 4A and 4B or can be designed to include one or more framework regions from another antibody or antibody fragment. For example, an Fab can be designed to include the six CDRs set forth in FIGS. 4A and 4B and the framework regions set forth in FIGS. 4A and 4B except that framework region 1 having the amino acid set forth in SEQ ID NO:36 is replaced with a framework region 1 having the amino acid set forth in SEQ ID NO:4 or a framework region 1 having the amino acid set forth in SEQ ID NO:20. In another example, an Fab can be designed to include the six CDRs set forth in FIGS. 4A and 4B and the framework regions set forth in FIGS. 4A and 4B except that framework region 1 having the amino acid set forth in SEQ ID NO:44 is replaced with a framework region 1 having the amino acid set forth in SEQ ID NO:12 or a framework region 1 having the amino acid set forth in SEQ ID NO:28. In another example, a scFv can be designed to include the six CDRs set forth in FIGS. 4A and 4B and the framework regions set forth in FIGS. 11A and 11B or the framework regions set forth in FIGS. 12A and 12B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:40 and/or (b) a light chain variable domain that includes an amino acid sequence having at least 90 percent identity to the amino acid sequence set forth in SEQ ID NO:48. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:40 and/or (b) a light chain variable domain that includes an amino acid sequence having at least 90, 91, 92, 93, 94, 95, 96, 97, 98, or 99 percent identity to the amino acid sequence set forth in SEQ ID NO:48. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:40 and/or (b) a light chain variable domain that includes an amino acid sequence having 100 percent identity to the amino acid sequence set forth in SEQ ID NO:48.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:40, provided that the heavy chain variable domain includes the amino acid sequences set forth in SEQ ID NOs:33, 34, and 35, and/or (b) a light chain variable domain that includes an amino acid sequence having at least 90 percent identity to the amino acid sequence set forth in SEQ ID NO:48, provided that the light chain variable domain includes the amino acid sequences set forth in SEQ ID NOs:41, 42, and 43. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:40, provided that the heavy chain variable domain includes the amino acid sequences set forth in SEQ ID NOs:33, 34, and 35, and/or (b) a light chain variable domain that includes an amino acid sequence having at least 90, 91, 92, 93, 94, 95, 96, 97, 98, or 99 percent identity to the amino acid sequence set forth in SEQ ID NO:48, provided that the light chain variable domain includes the amino acid sequences set forth in SEQ ID NOs:41, 42, and 43.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain having the amino acid sequence set forth in SEQ ID NO:40 or the amino acid set forth in SEQ ID NO:40 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:48 or the amino acid set forth in SEQ ID NO:48 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD276 polypeptide (e.g., a human CD276 polypeptide), can include a heavy chain variable domain having the amino acid sequence set forth in SEQ ID NO:40 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:33, 34, and 35, and can include a light chain variable domain having the amino acid sequence set forth in SEQ ID NO:48 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:41, 42, and 43.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain comprising (i) a CDR1 that comprises, consists essentially of, or consists of the amino acid sequence set forth in SEQ ID NO:33, (ii) a CDR2 that comprises, consists essentially of, or consists of the amino acid sequence set forth in SEQ ID NO:34, and (iii) a CDR3 that comprises, consists essentially of, or consists of the amino acid sequence set forth in SEQ ID NO:35 and/or (b) a light chain variable domain comprising (i) a CDR1 that comprises, consists essentially of, or consists of the amino acid sequence set forth in SEQ ID NO:41, (ii) a CDR2 that comprises, consists essentially of, or consists of the amino acid sequence set forth in SEQ ID NO:42, and (iii) a CDR3 that comprises, consists essentially of, or consists of the amino acid sequence set forth in SEQ ID NO:43. As used herein, a “CDR1 that consists essentially of the amino acid sequence set forth in SEQ ID NO:33” is a CDR1 that has zero, one, or two amino acid substitutions within SEQ ID NO:33, that has zero, one, two, three, four, or five amino acid residues directly preceding SEQ ID NO:33, and/or that has zero, one, two, three, four, or five amino acid residues directly following SEQ ID NO:33, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:33 include, without limitation, those set forth in Table 13.

TABLE 13
Exemplary CDR1s that consist essentially of the
amino acid sequence set forth in SEQ ID NO: 33.

| Sequence | SEQ ID NO: |
|---|---|
| GFTFDDYA | 251 |
| GFTFSSYD | 252 |
| GYTFTGYY | 253 |
| GFTFSSYW | 254 |
| GFTFSGSA | 255 |
| GFTFSSYA | 256 |
| GFTFGDYA | 257 |
| GFTVSSNE | 258 |
| GFTFSSYG | 259 |
| GFTFSYYY | 260 |

As used herein, a “CDR2 that consists essentially of the amino acid sequence set forth in SEQ ID NO:34” is a CDR2 that has zero, one, or two amino acid substitutions within SEQ ID NO:34, that has zero, one, two, three, four, or five amino acid residues directly preceding SEQ ID NO:34, and/or that has zero, one, two, three, four, or five amino acid residues directly following SEQ ID NO:34, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:34 include, without limitation, those set forth in Table 14.

TABLE 14
Exemplary CDR2s that consist essentially of the
amino acid sequence set forth in SEQ ID NO: 34.

| Sequence | SEQ ID NO: |
|---|---|
| IGTAGDT | 261 |
| IKQDGSEK | 262 |
| IKSKTDGGTT | 263 |
| ISGSGGST | 264 |
| ISYDGSNK | 265 |
| ISGGST | 266 |
| ISSSSSTI | 267 |
| ISWNSGSI | 268 |
| IKQDGSEK | 269 |
| ISAYNGNT | 270 |

As used herein, a “CDR3 that consists essentially of the amino acid sequence set forth in SEQ ID NO:35” is a CDR3 that has zero, one, or two amino acid substitutions within SEQ ID NO:35, that has zero, one, two, three, four, or five amino acid residues directly preceding SEQ ID NO:35, and/or that has zero, one, two, three, four, or five amino acid residues directly following SEQ ID NO:35, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:35 include, without limitation, those set forth in Table 15.

TABLE 15
Exemplary CDR3s that consist essentially of the
amino acid sequence set forth in SEQ ID NO: 35.

| Sequence | SEQ ID NO: |
|---|---|
| VRRGGSYYARGAFDI | 271 |
| SRRGGSYYARGAFDI | 272 |
| MRRGGSYYARGAFDI | 273 |
| TRRGGSYYARGAFDI | 274 |
| ARGGSYYARGAFDI | 275 |
| ARRGSYYARGAFDI | 276 |
| ARRGGSYARGAFDI | 277 |
| ARRGGSYYDRGAFDI | 278 |
| ARRGGSYYARGAFSI | 279 |
| ARRGGSYYARGYFDI | 280 |

As used herein, a “CDR1 that consists essentially of the amino acid sequence set forth in SEQ ID NO:41” is a CDR1 that has zero, one, or two amino acid substitutions within SEQ TD NO:41, that has zero, one, two, three, four, or five amino acid residues directly preceding SEQ ID NO:41, and/or that has zero, one, two, three, four, or five amino acid residues directly following SEQ ID NO:41, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:41 include, without limitation, those set forth in Table 16.

TABLE 16
Exemplary CDR1s that consist essentially of the
amino acid sequence set forth in SEQ ID NO: 41.

| Sequence | SEQ ID NO: |
|---|---|
| QSISSW | 281 |
| QGISSY | 282 |
| QGISNY | 283 |
| QGISSW | 284 |
| QGISSA | 285 |
| QSVSSY | 286 |
| QSVSSN | 287 |
| QSVSSSY | 288 |
| QSIGSS | 289 |
| QSVSSSY | 290 |

As used herein, a “CDR2 that consists essentially of the amino acid sequence set forth in SEQ ID NO:42” is a CDR2 that has zero or one amino acid substitutions within SEQ ID NO:42, that has zero, one, two, three, four, or five amino acid residues directly preceding SEQ ID NO:42, and/or that has zero, one, two, three, four, or five amino acid residues directly following SEQ ID NO:42, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:42 include, without limitation, those set forth in Table 17.

TABLE 17
Exemplary CDR2s that consist essentially of the
amino acid sequence set forth in SEQ ID NO: 42.

Sequence
DAS
SAS
DAK
YAS
TIS
RVS
KVS
LGS
EVS
EAT

As used herein, a “CDR3 that consists essentially of the amino acid sequence set forth in SEQ ID NO:43” is a CDR3 that has zero, one, or two amino acid substitutions within SEQ ID NO:43, that has zero, one, two, three, four, or five amino acid residues directly preceding SEQ ID NO:43, and/or that has zero, one, two, three, four, or five amino acid residues directly following SEQ ID NO:43, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:43 include, without limitation, those set forth in Table 18.

TABLE 18
Exemplary CDR3s that consist essentially of the
amino acid sequence set forth in SEQ ID NO: 43.

| Sequence | SEQ ID NO: |
|---|---|
| QQRSDWPPKFT | 301 |
| LQRSDWPPKFT | 302 |
| QKRSDWPPKFT | 303 |
| EQHRSDWPPKFT | 304 |
| MQHRSDWPPKFT | 305 |
| TQHRSDWPPKFT | 306 |
| QQHRSDWPPKFT | 307 |
| QHRTDWPPKFT | 308 |
| QHRSDWPPKFS | 309 |
| QHRSDWPPKDT | 310 |

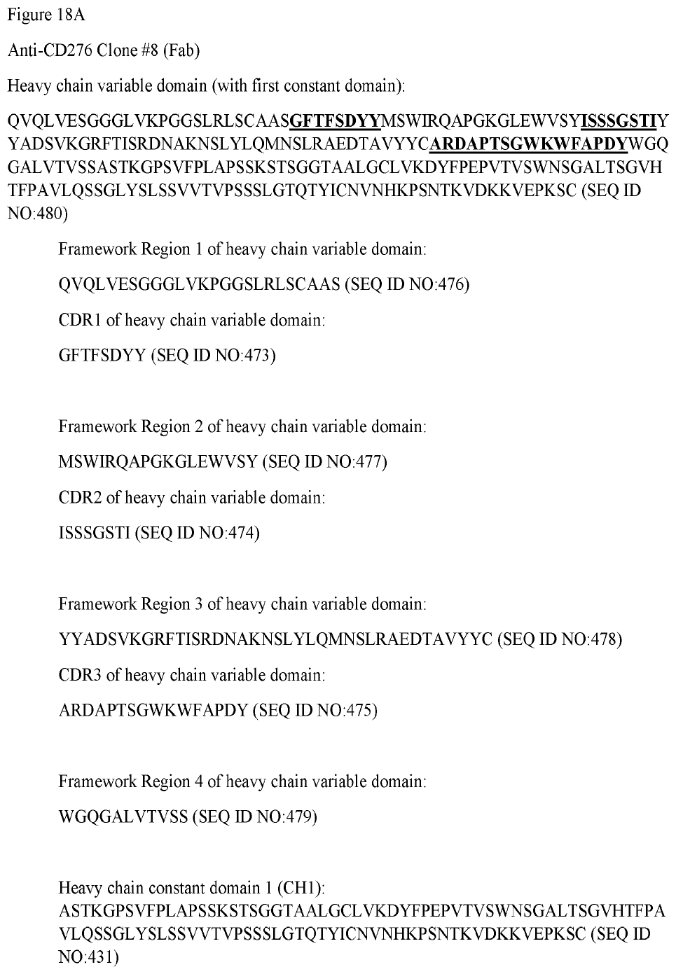

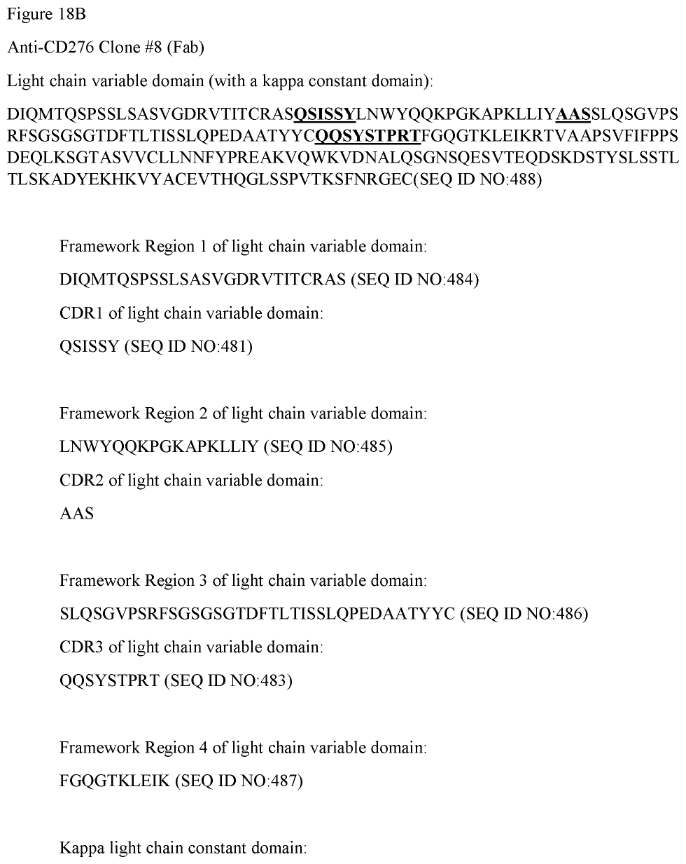

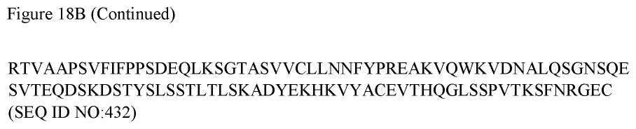

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (i) a heavy chain variable domain having a CDR1 having the amino acid sequence set forth in SEQ ID NO:473 (or a variant of SEQ ID NO:473 with one or two amino acid modifications), a CDR2 having the amino acid sequence set forth in SEQ ID NO:474 (or a variant of SEQ ID NO:474 with one or two amino acid modifications), and a CDR3 having the amino acid sequence set forth in SEQ ID NO:475 (or a variant of SEQ ID NO:475 with one or two amino acid modifications); and/or (ii) a light chain variable domain having a CDR1 having the amino acid sequence set forth in SEQ ID NO:481 (or a variant of SEQ ID NO:481 with one or two amino acid modifications), a CDR2 having the amino acid sequence set forth in SEQ ID NO:482 (or a variant of SEQ ID NO:482 with one or two amino acid modifications), and a CDR3 having the amino acid sequence set forth SEQ ID NO:483 (or a variant of SEQ ID NO:483 with one or two amino acid modifications). An example of such an antibody having these CDRs and the ability to bind to a CD276 polypeptide (e.g., a human CD276 polypeptide) includes, without limitation, the Fab set forth in FIGS. 18A and 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having (a) a heavy chain variable domain having a CDR1 having the amino acid sequence set forth in SEQ ID NO:473 (or a variant of SEQ ID NO:473 with one or two amino acid modifications), a CDR2 having the amino acid sequence set forth in SEQ ID NO:474 (or a variant of SEQ ID NO:474 with one or two amino acid modifications), and a CDR3 having the amino acid sequence set forth in SEQ ID NO:475 (or a variant of SEQ ID NO:475 with one or two amino acid modifications) and/or (b) a light chain variable domain having a CDR1 having the amino acid sequence set forth in SEQ ID NO:481 (or a variant of SEQ ID NO:481 with one or two amino acid modifications), a CDR2 having the amino acid sequence set forth in SEQ ID NO:482 (or a variant of SEQ ID NO:482 with one or two amino acid modifications), and a CDR3 having the amino acid sequence set forth SEQ ID NO:483 (or a variant of SEQ ID NO:483 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:476 (or a variant of SEQ ID NO:476 with one, two, three, four, five, six, seven, eight, nine, ten, or more amino acid modifications), a framework region 2 having the amino acid sequence set forth in SEQ ID NO:477 (or a variant of SEQ ID NO:477 with one, two, three, four, five, six, seven, eight, nine, ten, or more amino acid modifications), a framework region 3 having the amino acid sequence set forth in SEQ ID NO:478 (or a variant of SEQ ID NO:478 with one, two, three, four, five, six, seven, eight, nine, ten, or more amino acid modifications), and a framework region 4 having the amino acid sequence set forth in SEQ ID NO:479 (or a variant of SEQ ID NO:479 with one, two, three, four, five, six, seven, eight, nine, ten, or more amino acid modifications) and/or (b) a light chain variable domain that includes a framework region 1 having the amino acid sequence set forth in SEQ ID NO:484 (or a variant of SEQ ID NO:484 with one, two, three, four, five, six, seven, eight, nine, ten, or more amino acid modifications), a framework region 2 having the amino acid sequence set forth in SEQ ID NO:485 (or a variant of SEQ ID NO:485 with one, two, three, four, five, six, seven, eight, nine, ten, or more amino acid modifications), a framework region 3 having the amino acid sequence set forth in SEQ ID NO:486 (or a variant of SEQ ID NO:486 with one, two, three, four, five, six, seven, eight, nine, ten, or more amino acid modifications), and a framework region 4 having the amino acid sequence set forth in SEQ ID NO:487 (or a variant of SEQ ID NO:487 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 18A or 18B can be designed to include framework regions as set forth in FIGS. 18A and 18B or can be designed to include one or more framework regions from another antibody or antibody fragment. For example, an Fab can be designed to include the six CDRs set forth in FIGS. 18A and 18B and the framework regions set forth in FIGS. 18A and 18B except that framework region 1 having the amino acid set forth in SEQ ID NO:476 is replaced with a framework region 1 having the amino acid set forth in SEQ ID NO:4 or a framework region 1 having the amino acid set forth in SEQ ID NO:20. In another example, an Fab can be designed to include the six CDRs set forth in FIGS. 18A and 18B and the framework regions set forth in FIGS. 18A and 18B except that framework region 1 having the amino acid set forth in SEQ ID NO:484 is replaced with a framework region 1 having the amino acid set forth in SEQ ID NO:12 or a framework region 1 having the amino acid set forth in SEQ ID NO:28. In another example, a scFv can be designed to include the six CDRs set forth in FIGS. 18A and 18B and the framework regions set forth in FIGS. 11A and 11B or the framework regions set forth in FIGS. 12A and 12B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:480 and/or (b) a light chain variable domain that includes an amino acid sequence having at least 90 percent identity to the amino acid sequence set forth in SEQ ID NO:488. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:480 and/or (b) a light chain variable domain that includes an amino acid sequence having at least 90, 91, 92, 93, 94, 95, 96, 97, 98, or 99 percent identity to the amino acid sequence set forth in SEQ ID NO:488. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:480 and/or (b) a light chain variable domain that includes an amino acid sequence having 100 percent identity to the amino acid sequence set forth in SEQ ID NO:488.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain that includes an amino acid sequence having at least 90 percent identity to the amino acid sequence set forth in SEQ ID NO:480, provided that the heavy chain variable domain includes the amino acid sequences set forth in SEQ ID NOs:473, 474, and 475, and/or (b) a light chain variable domain that includes an amino acid sequence having at least 90 percent identity to the amino acid sequence set forth in SEQ ID NO:488, provided that the light chain variable domain includes the amino acid sequences set forth in SEQ ID NOs:481, 482, and 483. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:480, provided that the heavy chain variable domain includes the amino acid sequences set forth in SEQ ID NOs:473, 474, and 475, and/or (b) a light chain variable domain that includes an amino acid sequence having at least 90, 91, 92, 93, 94, 95, 96, 97, 98, or 99 percent identity to the amino acid sequence set forth in SEQ ID NO:488, provided that the light chain variable domain includes the amino acid sequences set forth in SEQ ID NOs:481, 482, and 483.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain having the amino acid sequence set forth in SEQ ID NO:480 or the amino acid set forth in SEQ ID NO:480 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:488 or the amino acid set forth in SEQ ID NO:488 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD276 polypeptide (e.g., a human CD276 polypeptide), can include a heavy chain variable domain having the amino acid sequence set forth in SEQ ID NO:480 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:473, 474, and 475, and can include a light chain variable domain having the amino acid sequence set forth in SEQ ID NO:488 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:481, 482, and 483.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include (a) a heavy chain variable domain comprising (i) a CDR1 that comprises, consists essentially of, or consists of the amino acid sequence set forth in SEQ ID NO:473, (ii) a CDR2 that comprises, consists essentially of, or consists of the amino acid sequence set forth in SEQ ID NO:474, and (iii) a CDR3 that comprises, consists essentially of, or consists of the amino acid sequence set forth in SEQ ID NO:475 and/or (b) a light chain variable domain comprising (i) a CDR1 that comprises, consists essentially of, or consists of the amino acid sequence set forth in SEQ ID NO:481, (ii) a CDR2 that comprises, consists essentially of, or consists of the amino acid sequence set forth in SEQ ID NO:482, and (iii) a CDR3 that comprises, consists essentially of, or consists of the amino acid sequence set forth in SEQ ID NO:483. As used herein, a “CDR1 that consists essentially of the amino acid sequence set forth in SEQ ID NO:473” is a CDR1 that has zero, one, or two amino acid substitutions within SEQ ID NO:473, that has zero, one, two, three, four, or five amino acid residues directly preceding SEQ ID NO:473, and/or that has zero, one, two, three, four, or five amino acid residues directly following SEQ ID NO:473, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:473 include, without limitation, those set forth in Table 19.

TABLE 19
Exemplary CDR1s that consist essentially of the
amino acid sequence set forth in SEQ ID NO: 473.

| Sequence | SEQ ID NO: |
|---|---|
| GFTFDDYA | 251 |
| GFTFSSYD | 252 |
| GYTFTGYY | 253 |
| GFTFSSYW | 254 |
| GFTFSGSA | 255 |
| GFTFSSYA | 256 |
| GFTFGDYA | 257 |
| GFTVSSNE | 258 |
| GFTFSSYG | 259 |
| GFTFSYYY | 260 |

As used herein, a “CDR2 that consists essentially of the amino acid sequence set forth in SEQ ID NO:474” is a CDR2 that has zero, one, or two amino acid substitutions within SEQ TD NO:474, that has zero, one, two, three, four, or five amino acid residues directly preceding SEQ TD NO:474, and/or that has zero, one, two, three, four, or five amino acid residues directly following SEQ ID NO:474, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:474 include, without limitation, those set forth in Table 20.

TABLE 20
Exemplary CDR2s that consist essentially of the
amino acid sequence set forth in SEQ ID NO: 474.

| Sequence | SEQ ID NO: |
|---|---|
| IGTAGDT | 261 |
| IKQDGSEK | 262 |
| IKSKTDGGTT | 263 |
| ISGSGGST | 264 |
| ISYDGSNK | 265 |
| ISGGST | 266 |
| ISSSSSTI | 267 |
| ISWNSGSI | 268 |
| IKQDGSEK | 269 |
| ISAYNGNT | 270 |

As used herein, a “CDR3 that consists essentially of the amino acid sequence set forth in SEQ ID NO:475” is a CDR3 that has zero, one, or two amino acid substitutions within SEQ TD NO:475, that has zero, one, two, three, four, or five amino acid residues directly preceding SEQ TD NO:475, and/or that has zero, one, two, three, four, or five amino acid residues directly following SEQ ID NO:475, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:475 include, without limitation, those set forth in Table 21.

TABLE 21
Exemplary CDR3s that consist essentially of the
amino acid sequence set forth in SEQ ID NO: 475.

| Sequence | SEQ ID NO: |
|---|---|
| ARDAPTSGWKWFYPDY | 491 |
| ARDYPTSGWKWFAPDY | 492 |
| ARDAPSSGWKWFAPDY | 493 |
| ARDAPTSGWKWFDPDY | 494 |
| ARDAPESGWKWFAPDY | 495 |
| ARDAPTSGWKWFSPDY | 496 |
| ARDAPTDGWKWFAPDY | 497 |
| ARDAPTSGWKWFAPDA | 498 |
| SRDAPTSGWKWFAPDY | 499 |
| ARDAPTSGWKWFAPDS | 500 |

As used herein, a “CDR1 that consists essentially of the amino acid sequence set forth in SEQ ID NO:481” is a CDR1 that has zero, one, or two amino acid substitutions within SEQ ID NO:481, that has zero, one, two, three, four, or five amino acid residues directly preceding SEQ ID NO:481, and/or that has zero, one, two, three, four, or five amino acid residues directly following SEQ ID NO:481, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:481 include, without limitation, those set forth in Table 22.

TABLE 22
Exemplary CDR1s that consist essentially of the
amino acid sequence set forth in SEQ ID NO: 481.

| Sequence | SEQ ID NO: |
|---|---|
| QSISSW | 281 |
| QGISSY | 282 |
| QGISNY | 283 |
| QGISSW | 284 |
| QGISSA | 285 |
| QSVSSY | 286 |
| QSVSSN | 287 |
| QSVSSSY | 288 |
| QSIGSS | 289 |
| QSVSSSY | 290 |

As used herein, a “CDR2 that consists essentially of the amino acid sequence set forth in SEQ ID NO:482” is a CDR2 that has zero or one amino acid substitutions within SEQ ID NO:482, that has zero, one, two, three, four, or five amino acid residues directly preceding SEQ ID NO:482, and/or that has zero, one, two, three, four, or five amino acid residues directly following SEQ ID NO:482, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:482 include, without limitation, those set forth in Table 23.

TABLE 23
Exemplary CDR2s that consist essentially of the
amino acid sequence set forth in SEQ ID NO: 482.

Sequence
DAS
SAS
DAK
YAS
TIS
RVS
KVS
LGS
EVS
EAT

As used herein, a “CDR3 that consists essentially of the amino acid sequence set forth in SEQ ID NO:483” is a CDR3 that has zero, one, or two amino acid substitutions within SEQ ID NO:483, that has zero, one, two, three, four, or five amino acid residues directly preceding SEQ ID NO:483, and/or that has zero, one, two, three, four, or five amino acid residues directly following SEQ ID NO:483, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:483 include, without limitation, those set forth in Table 24.

TABLE 24
Exemplary CDR3s that consist essentially of the
amino acid sequence set forth in SEQ ID NO: 483.

| Sequence | SEQ ID NO: |
|---|---|
| QQSYSEPRT | 501 |
| QQSYSTPRE | 502 |
| QQAYSTPRT | 503 |
| QQSYDTPRT | 504 |
| QQDYSTPRT | 505 |
| QQSYATPRT | 506 |
| QQAYSTPRE | 507 |
| QQSYDTPRE | 508 |
| QQYYSTPRT | 509 |
| QQSASTPRT | 510 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3; SEQ ID NOs:17-19; SEQ ID NOs:33-35; or SEQ ID NOs:473-475 can be directly connected to a light chain variable domain having the CDRs of SEQ ID NOs:9-11; SEQ ID NOs:25-27; SEQ ID NOs:41-43; or SEQ ID NOs:481-483, respectively, via a linker sequence. Examples of linker sequences that can be used to connect a heavy chain variable domain and a light chain variable domain to create a scFv include, without limitation, those linkers set forth in FIG. 10.

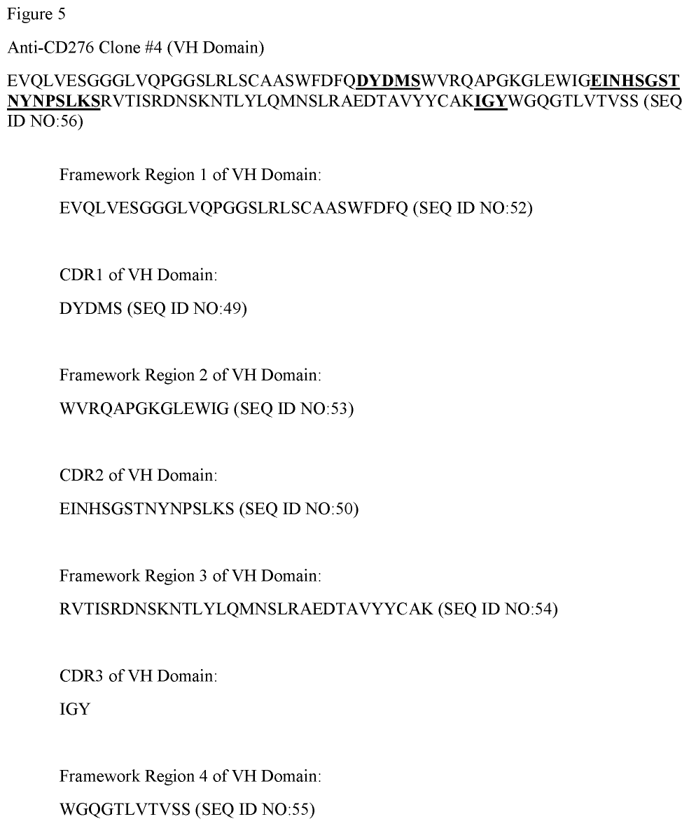

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having a CDR1 having the amino acid sequence set forth in SEQ ID NO:49 (or a variant of SEQ ID NO:49 with one or two amino acid modifications), a CDR2 having the amino acid sequence set forth in SEQ ID NO:50 (or a variant of SEQ ID NO:50 with one or two amino acid modifications), and a CDR3 having the amino acid sequence set forth in SEQ ID NO:51 (or a variant of SEQ ID NO:51 with one or two amino acid modifications). An example of such an antibody domain having these CDRs and the ability to bind to a CD276 polypeptide (e.g., a human CD276 polypeptide) includes, without limitation, the VH domain set forth in FIG. 5.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having a heavy chain variable domain having a CDR1 having the amino acid sequence set forth in SEQ ID NO:49 (or a variant of SEQ ID NO:49 with one or two amino acid modifications), a CDR2 having the amino acid sequence set forth in SEQ ID NO:50 (or a variant of SEQ ID NO:50 with one or two amino acid modifications), and a CDR3 having the amino acid sequence set forth in SEQ ID NO:51 (or a variant of SEQ ID NO:51 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:52 (or a variant of SEQ ID NO:52 with one, two, three, four, five, six, seven, eight, nine, ten, or more amino acid modifications), a framework region 2 having the amino acid sequence set forth in SEQ ID NO:53 (or a variant of SEQ ID NO:53 with one, two, three, four, five, six, seven, eight, nine, ten, or more amino acid modifications), a framework region 3 having the amino acid sequence set forth in SEQ ID NO:54 (or a variant of SEQ ID NO:54 with one, two, three, four, five, six, seven, eight, nine, ten, or more amino acid modifications), and a framework region 4 having the amino acid sequence set forth in SEQ ID NO:55 (or a variant of SEQ ID NO:55 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 5 can be designed to include framework regions as set forth in FIG. 5 or can be designed to include one or more framework regions from another antibody or antibody fragment. For example, an antibody domain (e.g., a VH domain) can be designed to include the three CDRs set forth in FIG. 5 and the framework regions set forth in FIG. 5 except that framework region 1 having the amino acid set forth in SEQ ID NO:52 is replaced with a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:532, or a framework region 1 having the amino acid set forth in SEQ ID NO:540. In another example, an Fab or scFv can be designed to include (a) the three CDRs set forth in FIG. 5, (b) the framework regions set forth in FIG. 5, 6, 7, 8, 26, or 27, (c) three CDRs of a light chain variable domain provided herein (e.g., SEQ ID NOs:9-11; SEQ ID NOs:25-27; SEQ ID NOs:41-43; or SEQ ID NOs:481-483), and (d) the framework regions of a light chain variable domain set forth in FIG. 2B, 3B, 4B, or 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:56. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:56. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:56.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:56, provided that the heavy chain variable domain includes the amino acid sequences set forth in SEQ ID NOs:49, 50, and 51. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:56, provided that the heavy chain variable domain includes the amino acid sequences set forth in SEQ ID NOs:49, 50, and 51.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having the amino acid sequence set forth in SEQ ID NO:56 or the amino acid set forth in SEQ ID NO:56 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD276 polypeptide (e.g., a human CD276 polypeptide) and can include a heavy chain variable domain having the amino acid sequence set forth in SEQ ID NO:56 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:49, 50, and 51.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain comprising (i) a CDR1 that comprises, consists essentially of, or consists of the amino acid sequence set forth in SEQ ID NO:49, (ii) a CDR2 that comprises, consists essentially of, or consists of the amino acid sequence set forth in SEQ ID NO:50, and (iii) a CDR3 that comprises, consists essentially of, or consists of the amino acid sequence set forth in SEQ ID NO:51. As used herein, a “CDR1 that consists essentially of the amino acid sequence set forth in SEQ ID NO:49” is a CDR1 that has zero, one, or two amino acid substitutions within SEQ ID NO:49, that has zero, one, two, three, four, or five amino acid residues directly preceding SEQ ID NO:49, and/or that has zero, one, two, three, four, or five amino acid residues directly following SEQ ID NO:49, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:49 include, without limitation, those set forth in Table 25.

TABLE 25
Exemplary CDR1s that consist essentially of the
amino acid sequence set forth in SEQ ID NO: 49.

| Sequence | SEQ ID NO: |
|---|---|
| DYDMH | 311 |
| DYDMG | 312 |
| DYAMS | 313 |
| DYYMS | 314 |
| SYDMS | 315 |
| DYYMH | 316 |
| DYYMN | 317 |
| SYDMN | 318 |
| SYDMH | 319 |
| DYDMD | 320 |

As used herein, a “CDR2 that consists essentially of the amino acid sequence set forth in SEQ ID NO:50” is a CDR2 that has zero, one, or two amino acid substitutions within SEQ ID NO:50, that has zero, one, two, three, four, or five amino acid residues directly preceding SEQ ID NO:50, and/or that has zero, one, two, three, four, or five amino acid residues directly following SEQ ID NO:50, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:50 include, without limitation, those set forth in Table 26.

TABLE 26
Exemplary CDR2s that consist essentially of the
amino acid sequence set forth in SEQ ID NO: 50.

| Sequence | SEQ ID NO: |
|---|---|
| DINHSGSTNYNPSLKS | 321 |
| EINHSGSTNYNPSLRS | 322 |
| EINHSGSTNYNPSLKG | 323 |
| EINHSGSTYYNPSLKS | 324 |
| EINHSGSTYYNPSLKG | 325 |
| EINHSGSTNYNPSVKS | 326 |
| EINHSGSTNYNPSVKG | 327 |
| EIYHSGSTNYNPSLKS | 328 |
| EIYHSGSTNYNPSLKG | 329 |
| DIYHSGSTNYNPSLKG | 330 |

As used herein, a “CDR3 that consists essentially of the amino acid sequence set forth in SEQ ID NO:51” is a CDR3 that has zero, one, or two amino acid substitutions within SEQ TD NO:51, that has zero, one, two, three, four, or five amino acid residues directly preceding SEQ ID NO:51, and/or that has zero, one, two, three, four, or five amino acid residues directly following SEQ ID NO:51, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO: 51 include, without limitation, those set forth in Table 27.

TABLE 27
Exemplary CDR3s that consist essentially of the
amino acid sequence set forth in SEQ ID NO: 51.

Sequence
IDY
LGY
ISY
LSY
LDY
IGF
ISF
LSF
FDY
FSY

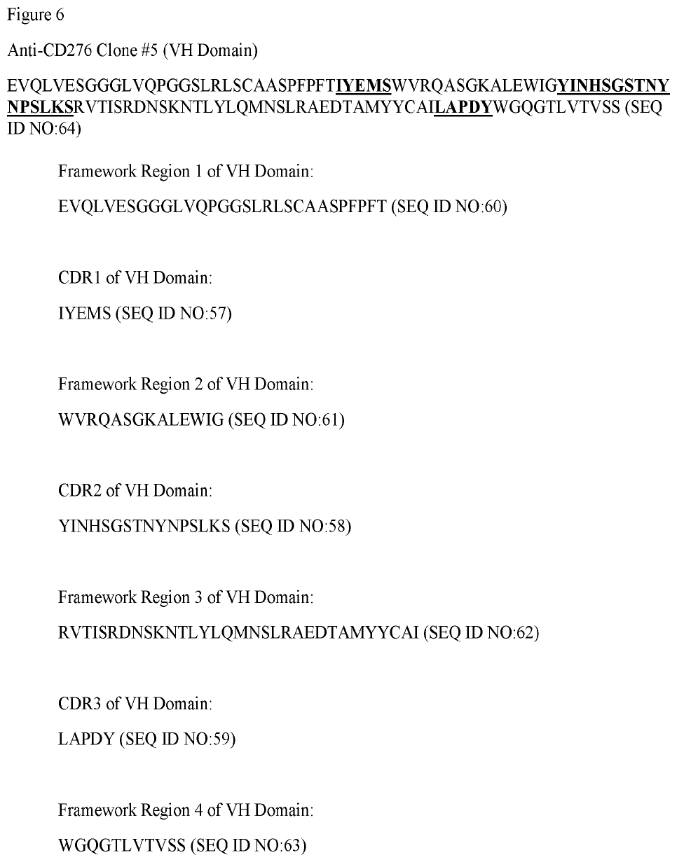

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having a CDR1 having the amino acid sequence set forth in SEQ ID NO:57 (or a variant of SEQ ID NO:57 with one or two amino acid modifications), a CDR2 having the amino acid sequence set forth in SEQ ID NO:58 (or a variant of SEQ ID NO:58 with one or two amino acid modifications), and a CDR3 having the amino acid sequence set forth in SEQ ID NO:59 (or a variant of SEQ ID NO:59 with one or two amino acid modifications). An example of such an antibody domain having these CDRs and the ability to bind to a CD276 polypeptide (e.g., a human CD276 polypeptide) includes, without limitation, the VH domain set forth in FIG. 6.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having a heavy chain variable domain having a CDR1 having the amino acid sequence set forth in SEQ ID NO:57 (or a variant of SEQ ID NO:57 with one or two amino acid modifications), a CDR2 having the amino acid sequence set forth in SEQ ID NO:58 (or a variant of SEQ ID NO:58 with one or two amino acid modifications), and a CDR3 having the amino acid sequence set forth in SEQ ID NO:59 (or a variant of SEQ ID NO:59 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:60 (or a variant of SEQ ID NO:60 with one, two, three, four, five, six, seven, eight, nine, ten, or more amino acid modifications), a framework region 2 having the amino acid sequence set forth in SEQ ID NO:61 (or a variant of SEQ ID NO:61 with one, two, three, four, five, six, seven, eight, nine, ten, or more amino acid modifications), a framework region 3 having the amino acid sequence set forth in SEQ ID NO:62 (or a variant of SEQ ID NO:62 with one, two, three, four, five, six, seven, eight, nine, ten, or more amino acid modifications), and a framework region 4 having the amino acid sequence set forth in SEQ ID NO:63 (or a variant of SEQ ID NO:63 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 6 can be designed to include framework regions as set forth in FIG. 6 or can be designed to include one or more framework regions from another antibody or antibody fragment. For example, an antibody domain (e.g., a VH domain) can be designed to include the three CDRs set forth in FIG. 6 and the framework regions set forth in FIG. 6 except that framework region 1 having the amino acid set forth in SEQ ID NO:60 is replaced with a framework region 1 having the amino acid set forth in SEQ ID NO:52, a framework region 1 having the amino acid set forth in SEQ ID NO:68, a framework region 1 having the amino acid set forth in SEQ ID NO:76, a framework region 1 having the amino acid set forth in SEQ ID NO:532, or a framework region 1 having the amino acid set forth in SEQ ID NO:540. In another example, an Fab or scFv can be designed to include (a) the three CDRs set forth in FIG. 6, (b) the framework regions set forth in FIG. 5, 6, 7, 8, 26, or 27, (c) three CDRs of a light chain variable domain provided herein (e.g., SEQ ID NOs:9-11; SEQ ID NOs:25-27; SEQ ID NOs:41-43; or SEQ ID NOs:481-483), and (d) the framework regions of a light chain variable domain set forth in FIG. 2B, 3B, 4B, or 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:64. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:64. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:64.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:64, provided that the heavy chain variable domain includes the amino acid sequences set forth in SEQ ID NOs:57, 58, and 59. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:64, provided that the heavy chain variable domain includes the amino acid sequences set forth in SEQ ID NOs:57, 58, and 59.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having the amino acid sequence set forth in SEQ ID NO:64 or the amino acid set forth in SEQ ID NO:64 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD276 polypeptide (e.g., a human CD276 polypeptide) and can include a heavy chain variable domain having the amino acid sequence set forth in SEQ ID NO:64 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:57, 58, and 59.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain comprising (i) a CDR1 that comprises, consists essentially of, or consists of the amino acid sequence set forth in SEQ ID NO:57, (ii) a CDR2 that comprises, consists essentially of, or consists of the amino acid sequence set forth in SEQ ID NO:58, and (iii) a CDR3 that comprises, consists essentially of, or consists of the amino acid sequence set forth in SEQ ID NO:59. As used herein, a “CDR1 that consists essentially of the amino acid sequence set forth in SEQ ID NO:57” is a CDR1 that has zero, one, or two amino acid substitutions within SEQ ID NO:57, that has zero, one, two, three, four, or five amino acid residues directly preceding SEQ ID NO:57, and/or that has zero, one, two, three, four, or five amino acid residues directly following SEQ ID NO:57, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:57 include, without limitation, those set forth in Table 28.

TABLE 28
Exemplary CDR1s that consist essentially of the
amino acid sequence set forth in SEQ ID NO: 57.

| Sequence | SEQ ID NO: |
|---|---|
| IYEMH | 341 |
| IYEMD | 342 |
| IYEMG | 343 |
| IYEMN | 344 |
| IYGMS | 345 |
| IYGMH | 346 |
| IYGMD | 347 |
| IYGMG | 348 |
| IYGMN | 349 |
| LYEMS | 350 |

As used herein, a “CDR2 that consists essentially of the amino acid sequence set forth in SEQ ID NO:58” is a CDR2 that has zero, one, or two amino acid substitutions within SEQ TD NO: 58, that has zero, one, two, three, four, or five amino acid residues directly preceding SEQ ID NO:58, and/or that has zero, one, two, three, four, or five amino acid residues directly following SEQ ID NO:58, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:58 include, without limitation, those set forth in Table 29.

TABLE 29
Exemplary CDR2s that consist essentially of the
amino acid sequence set forth in SEQ ID NO: 58.

| Sequence | SEQ ID NO: |
|---|---|
| YIYHSGSTNYNPSLKS | 351 |
| YINHSGSTNYNPSLKG | 352 |
| YINHSGSTNYNPSVKS | 353 |
| YIYHSGSTNYNPSLKG | 354 |
| YIYHSGSTNYNPSVKS | 355 |
| YIYHSGSTYYNPSLKS | 356 |
| YINHSGSTYYNPSLKS | 357 |
| YINHSGSTYYNPSLKG | 358 |
| YINHSGSTYYNPSVKS | 359 |
| YINHGGSTNYNPSLKS | 360 |

As used herein, a “CDR3 that consists essentially of the amino acid sequence set forth in SEQ ID NO:59” is a CDR3 that has zero, one, or two amino acid substitutions within SEQ ID NO:59, that has zero, one, two, three, four, or five amino acid residues directly preceding SEQ ID NO:59, and/or that has zero, one, two, three, four, or five amino acid residues directly following SEQ ID NO:59, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:59 include, without limitation, those set forth in Table 30.

TABLE 30
Exemplary CDR3s that consist essentially of the
amino acid sequence set forth in SEQ ID NO: 59.

| Sequence | SEQ ID NO: |
|---|---|
| LAPEY | 361 |
| IAPEY | 362 |
| LAPDY | 363 |
| LVPDY | 364 |
| IVPDY | 365 |
| LVPEY | 366 |
| VAPDY | 367 |
| VAPEY | 368 |
| VVPDY | 369 |
| LIPDY | 370 |

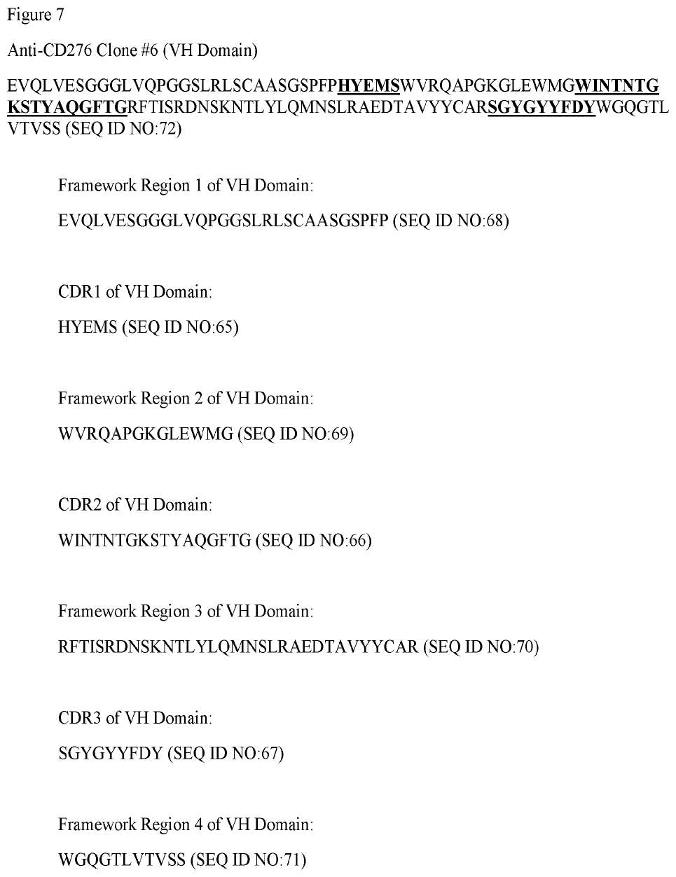

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having a CDR1 having the amino acid sequence set forth in SEQ ID NO:65 (or a variant of SEQ ID NO:65 with one or two amino acid modifications), a CDR2 having the amino acid sequence set forth in SEQ ID NO:66 (or a variant of SEQ ID NO:66 with one or two amino acid modifications), and a CDR3 having the amino acid sequence set forth in SEQ ID NO:67 (or a variant of SEQ ID NO:67 with one or two amino acid modifications). An example of such an antibody domain having these CDRs and the ability to bind to a CD276 polypeptide (e.g., a human CD276 polypeptide) includes, without limitation, the VH domain set forth in FIG. 7.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having a heavy chain variable domain having a CDR1 having the amino acid sequence set forth in SEQ ID NO:65 (or a variant of SEQ ID NO:65 with one or two amino acid modifications), a CDR2 having the amino acid sequence set forth in SEQ ID NO:66 (or a variant of SEQ ID NO:66 with one or two amino acid modifications), and a CDR3 having the amino acid sequence set forth in SEQ ID NO:67 (or a variant of SEQ ID NO:67 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:68 (or a variant of SEQ ID NO:68 with one, two, three, four, five, six, seven, eight, nine, ten, or more amino acid modifications), a framework region 2 having the amino acid sequence set forth in SEQ ID NO:69 (or a variant of SEQ ID NO:69 with one, two, three, four, five, six, seven, eight, nine, ten, or more amino acid modifications), a framework region 3 having the amino acid sequence set forth in SEQ ID NO:70 (or a variant of SEQ ID NO:70 with one, two, three, four, five, six, seven, eight, nine, ten, or more amino acid modifications), and a framework region 4 having the amino acid sequence set forth in SEQ ID NO:71 (or a variant of SEQ ID NO:71 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 7 can be designed to include framework regions as set forth in FIG. 7 or can be designed to include one or more framework regions from another antibody or antibody fragment. For example, an antibody domain (e.g., a VH domain) can be designed to include the three CDRs set forth in FIG. 7 and the framework regions set forth in FIG. 7 except that framework region 1 having the amino acid set forth in SEQ ID NO:68 is replaced with a framework region 1 having the amino acid set forth in SEQ ID NO:52, a framework region 1 having the amino acid set forth in SEQ ID NO:60, a framework region 1 having the amino acid set forth in SEQ ID NO:76, a framework region 1 having the amino acid set forth in SEQ ID NO:532, or a framework region 1 having the amino acid set forth in SEQ ID NO:540. In another example, an Fab or scFv can be designed to include (a) the three CDRs set forth in FIG. 7, (b) the framework regions set forth in FIG. 5, 6, 7, 8, 26, or 27, (c) three CDRs of a light chain variable domain provided herein (e.g., SEQ ID NOs:9-11; SEQ ID NOs:25-27; SEQ ID NOs:41-43; or SEQ ID NOs:481-483), and (d) the framework regions of a light chain variable domain set forth in FIG. 2B, 3B, 4B, or 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:72. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:72. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:72.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:72, provided that the heavy chain variable domain includes the amino acid sequences set forth in SEQ ID NOs:65, 66, and 67. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:72, provided that the heavy chain variable domain includes the amino acid sequences set forth in SEQ ID NOs:65, 66, and 67.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having the amino acid sequence set forth in SEQ ID NO:72 or the amino acid set forth in SEQ ID NO:72 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a CD276 polypeptide (e.g., a human CD276 polypeptide) and can include a heavy chain variable domain having the amino acid sequence set forth in SEQ ID NO:72 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:65, 66, and 67.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain comprising (i) a CDR1 that comprises, consists essentially of, or consists of the amino acid sequence set forth in SEQ ID NO:65, (ii) a CDR2 that comprises, consists essentially of, or consists of the amino acid sequence set forth in SEQ ID NO:66, and (iii) a CDR3 that comprises, consists essentially of, or consists of the amino acid sequence set forth in SEQ ID NO:67. As used herein, a “CDR1 that consists essentially of the amino acid sequence set forth in SEQ ID NO:65” is a CDR1 that has zero, one, or two amino acid substitutions within SEQ ID NO:65, that has zero, one, two, three, four, or five amino acid residues directly preceding SEQ ID NO:65, and/or that has zero, one, two, three, four, or five amino acid residues directly following SEQ ID NO:65, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:65 include, without limitation, those set forth in Table 31.

TABLE 31
Exemplary CDR1s that consist essentially of the
amino acid sequence set forth in SEQ ID NO: 65.

| Sequence | SEQ ID NO: |
|---|---|
| HYDMS | 371 |
| HYEMH | 372 |
| HYEMG | 373 |
| HYEMD | 374 |
| HYEMN | 375 |
| HYDMG | 376 |
| HYDMH | 377 |
| HYDMD | 378 |
| HYDMN | 379 |
| DYEMG | 380 |

As used herein, a “CDR2 that consists essentially of the amino acid sequence set forth in SEQ ID NO:66” is a CDR2 that has zero, one, or two amino acid substitutions within SEQ ID NO:66, that has zero, one, two, three, four, or five amino acid residues directly preceding SEQ ID NO:66, and/or that has zero, one, two, three, four, or five amino acid residues directly following SEQ ID NO:66, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:66 include, without limitation, those set forth in Table 32.

TABLE 32
Exemplary CDR2s that consist essentially of the
amino acid sequence set forth in SEQ ID NO: 66.

| Sequence | SEQ ID NO: |
|---|---|
| WINPNTGKSTYAQGFTG | 381 |
| WINTNTGKSTYAQGFQG | 382 |
| WINTNTGKSTYAQKFTG | 383 |
| WINTNTGKSTYSQGFTG | 384 |
| WINTNTGKSNYAQGFTG | 385 |
| WINTNSGKSTYAQGFTG | 386 |
| WINTNTNKSTYAQGFTG | 387 |
| WLNTNTGKSTYAQGFTG | 388 |
| WINTNTGKSTYAQGFTS | 389 |
| WINTNTGKSTYANGFTG | 390 |

As used herein, a “CDR3 that consists essentially of the amino acid sequence set forth in SEQ ID NO:67” is a CDR3 that has zero, one, or two amino acid substitutions within SEQ ID NO:67, that has zero, one, two, three, four, or five amino acid residues directly preceding SEQ ID NO:67, and/or that has zero, one, two, three, four, or five amino acid residues directly following SEQ ID NO:67, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:67 include, without limitation, those set forth in Table 33.

TABLE 33
Exemplary CDR3s that consist essentially of the
amino acid sequence set forth in SEQ ID NO: 67.

| Sequence | SEQ ID NO: |
|---|---|
| TGYGYYFDY | 391 |
| SGYGYYFEY | 392 |
| SSYGYYFDY | 393 |
| SGYSYYFDY | 394 |
| SSYGYYFEY | 395 |
| SGYSYYFEY | 396 |
| TGYSYYFEY | 397 |
| TSYSYYFEY | 398 |
| TTYSYYFEY | 399 |
| TTYSYYFDY | 400 |

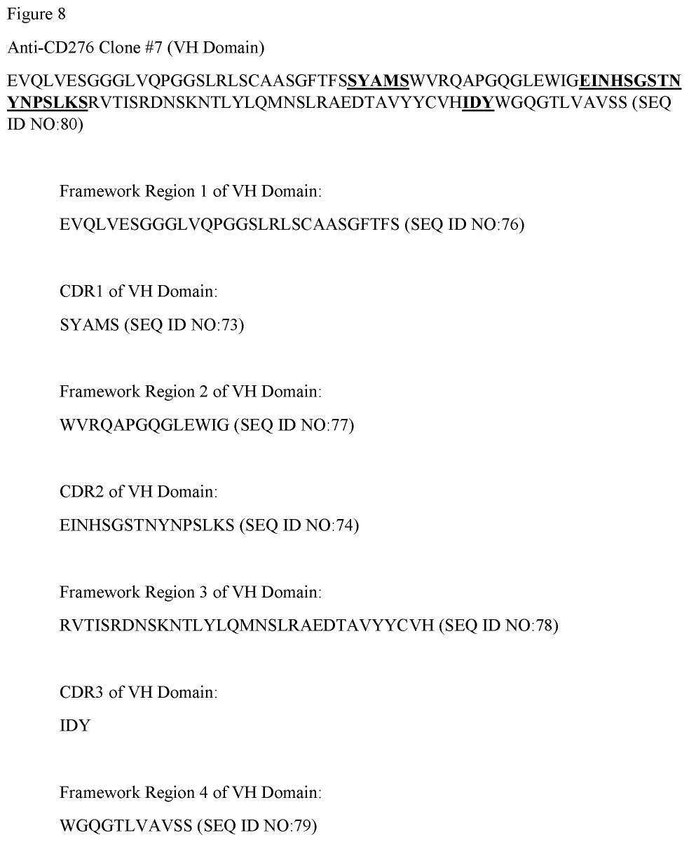

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having a CDR1 having the amino acid sequence set forth in SEQ ID NO:73 (or a variant of SEQ ID NO:73 with one or two amino acid modifications), a CDR2 having the amino acid sequence set forth in SEQ ID NO:74 (or a variant of SEQ ID NO:74 with one or two amino acid modifications), and a CDR3 having the amino acid sequence set forth in SEQ ID NO:75 (or a variant of SEQ ID NO:75 with one or two amino acid modifications). An example of such an antibody domain having these CDRs and the ability to bind to a CD276 polypeptide (e.g., a human CD276 polypeptide) includes, without limitation, the VH domain set forth in FIG. 8.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having a heavy chain variable domain having a CDR1 having the amino acid sequence set forth in SEQ ID NO:73 (or a variant of SEQ ID NO:73 with one or two amino acid modifications), a CDR2 having the amino acid sequence set forth in SEQ ID NO:74 (or a variant of SEQ ID NO:74 with one or two amino acid modifications), and a CDR3 having the amino acid sequence set forth in SEQ ID NO:75 (or a variant of SEQ ID NO:75 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:76 (or a variant of SEQ ID NO:76 with one, two, three, four, five, six, seven, eight, nine, ten, or more amino acid modifications), a framework region 2 having the amino acid sequence set forth in SEQ ID NO:77 (or a variant of SEQ ID NO:77 with one, two, three, four, five, six, seven, eight, nine, ten, or more amino acid modifications), a framework region 3 having the amino acid sequence set forth in SEQ ID NO:78 (or a variant of SEQ ID NO:78 with one, two, three, four, five, six, seven, eight, nine, ten, or more amino acid modifications), and a framework region 4 having the amino acid sequence set forth in SEQ ID NO:79 (or a variant of SEQ ID NO:79 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8 can be designed to include framework regions as set forth in FIG. 8 or can be designed to include one or more framework regions from another antibody or antibody fragment. For example, an antibody domain (e.g., a VH domain) can be designed to include the three CDRs set forth in FIG. 8 and the framework regions set forth in FIG. 8 except that framework region 1 having the amino acid set forth in SEQ ID NO:76 is replaced with a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:532, or a framework region 1 having the amino acid set forth in SEQ ID NO:540. In another example, an Fab or scFv can be designed to include (a) the three CDRs set forth in FIG. 8, (b) the framework regions set forth in FIG. 5, 6, 7, 8, 26, or 27, (c) three CDRs of a light chain variable domain provided herein (e.g., SEQ ID NOs:9-11; SEQ ID NOs:25-27; SEQ ID NOs:41-43; or SEQ ID NOs:481-483), and (d) the framework regions of a light chain variable domain set forth in FIG. 2B, 3B, 4B, or 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:80. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:80. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:80.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:80, provided that the heavy chain variable domain includes the amino acid sequences set forth in SEQ ID NOs:73, 74, and 75. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:80, provided that the heavy chain variable domain includes the amino acid sequences set forth in SEQ ID NOs:73, 74, and 75.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having the amino acid sequence set forth in SEQ ID NO:80 or the amino acid set forth in SEQ ID NO:80 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a CD276 polypeptide (e.g., a human CD276 polypeptide) and can include a heavy chain variable domain having the amino acid sequence set forth in SEQ ID NO:80 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:73, 74, and 75.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain comprising (i) a CDR1 that comprises, consists essentially of, or consists of the amino acid sequence set forth in SEQ ID NO:73, (ii) a CDR2 that comprises, consists essentially of, or consists of the amino acid sequence set forth in SEQ ID NO:74, and (iii) a CDR3 that comprises, consists essentially of, or consists of the amino acid sequence set forth in SEQ ID NO:75. As used herein, a “CDR1 that consists essentially of the amino acid sequence set forth in SEQ ID NO:73” is a CDR1 that has zero, one, or two amino acid substitutions within SEQ ID NO:73, that has zero, one, two, three, four, or five amino acid residues directly preceding SEQ ID NO:73, and/or that has zero, one, two, three, four, or five amino acid residues directly following SEQ ID NO:73, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:73 include, without limitation, those set forth in Table 34.

TABLE 34
Exemplary CDR1s that consist essentially of the
amino acid sequence set forth in SEQ ID NO: 73.

| Sequence | SEQ ID NO: |
|---|---|
| SYAMG | 401 |
| SYAMD | 402 |
| SYAMN | 403 |
| SYAMH | 404 |
| SYEMS | 405 |
| SYEMG | 406 |
| SYEMH | 407 |
| SYEMD | 408 |
| TYAMS | 409 |
| SYEMD | 410 |

As used herein, a “CDR2 that consists essentially of the amino acid sequence set forth in SEQ ID NO:74” is a CDR2 that has zero, one, or two amino acid substitutions within SEQ TD NO:74, that has zero, one, two, three, four, or five amino acid residues directly preceding SEQ ID NO:74, and/or that has zero, one, two, three, four, or five amino acid residues directly following SEQ ID NO:74, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO: 74 include, without limitation, those set forth in Table 35.

TABLE 35
Exemplary CDR2s that consist essentially of the
amino acid sequence set forth in SEQ ID NO: 74.

| Sequence | SEQ ID NO: |
|---|---|
| EINHNGSTNYNPSLKS | 411 |
| EINHSGSTNYNPSLKG | 412 |
| EINHSGSTNYNPSVKS | 413 |
| EINHSGSTYYNPSLKS | 414 |
| DINHSGSTNYNPSLKS | 415 |
| EINHSGTTNYNPSLKS | 416 |
| DINHSGTTNYNPSLKS | 417 |
| EINHSGTTNYNPSLKG | 418 |
| EINHSGTSNYNPSLKS | 419 |
| EINHSGTSNYNPSLKG | 420 |

As used herein, a “CDR3 that consists essentially of the amino acid sequence set forth in SEQ ID NO:75” is a CDR3 that has zero, one, or two amino acid substitutions within SEQ TD NO:75, that has zero, one, two, three, four, or five amino acid residues directly preceding SEQ ID NO: 75, and/or that has zero, one, two, three, four, or five amino acid residues directly following SEQ ID NO:75, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:75 include, without limitation, those set forth in Table 36.

TABLE 36
Exemplary CDR3s that consist essentially of the
amino acid sequence set forth in SEQ ID NO: 75.

Sequence
LDY
VDY
IEY
LEY
VEY
VEF
LEF
IDF
LDF
VDF

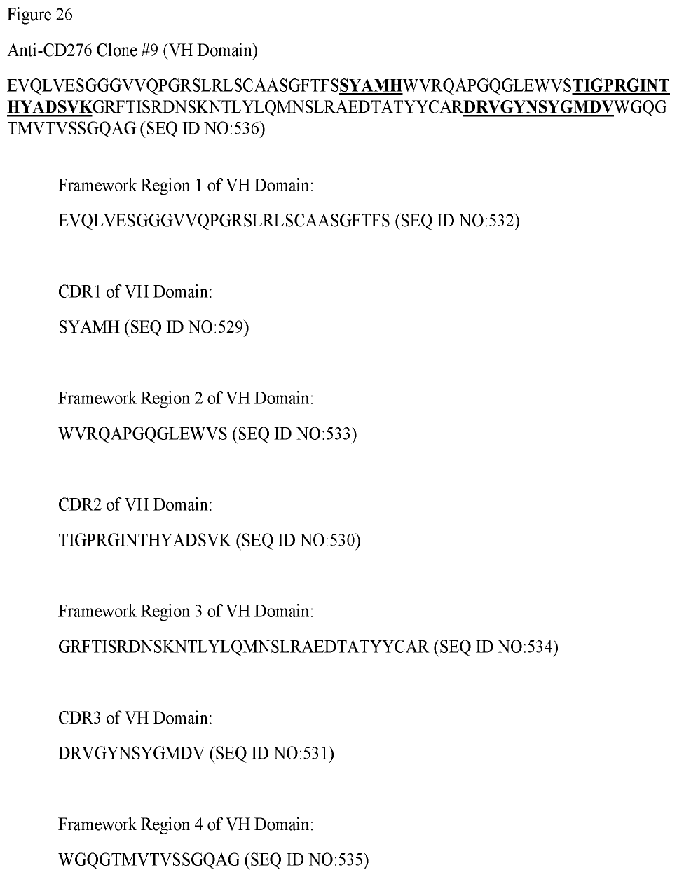

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having a CDR1 having the amino acid sequence set forth in SEQ ID NO:529 (or a variant of SEQ ID NO:529 with one or two amino acid modifications), a CDR2 having the amino acid sequence set forth in SEQ ID NO:530 (or a variant of SEQ ID NO:530 with one or two amino acid modifications), and a CDR3 having the amino acid sequence set forth in SEQ ID NO:531 (or a variant of SEQ ID NO:531 with one or two amino acid modifications). An example of such an antibody domain having these CDRs and the ability to bind to a CD276 polypeptide (e.g., a human CD276 polypeptide) includes, without limitation, the VH domain set forth in FIG. 26.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having a heavy chain variable domain having a CDR1 having the amino acid sequence set forth in SEQ ID NO:529 (or a variant of SEQ ID NO:529 with one or two amino acid modifications), a CDR2 having the amino acid sequence set forth in SEQ ID NO:530 (or a variant of SEQ ID NO:530 with one or two amino acid modifications), and a CDR3 having the amino acid sequence set forth in SEQ ID NO:531 (or a variant of SEQ ID NO:531 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:532 (or a variant of SEQ ID NO:532 with one, two, three, four, five, six, seven, eight, nine, ten, or more amino acid modifications), a framework region 2 having the amino acid sequence set forth in SEQ ID NO:533 (or a variant of SEQ ID NO:533 with one, two, three, four, five, six, seven, eight, nine, ten, or more amino acid modifications), a framework region 3 having the amino acid sequence set forth in SEQ ID NO:534 (or a variant of SEQ ID NO:534 with one, two, three, four, five, six, seven, eight, nine, ten, or more amino acid modifications), and a framework region 4 having the amino acid sequence set forth in SEQ ID NO:535 (or a variant of SEQ ID NO:535 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8 can be designed to include framework regions as set forth in FIG. 26 or can be designed to include one or more framework regions from another antibody or antibody fragment. For example, an antibody domain (e.g., a VH domain) can be designed to include the three CDRs set forth in FIG. 26 and the framework regions set forth in FIG. 26 except that framework region 1 having the amino acid set forth in SEQ ID NO:532 is replaced with a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, or a framework region 1 having the amino acid set forth in SEQ ID NO:540. In another example, an Fab or scFv can be designed to include (a) the three CDRs set forth in FIG. 26, (b) the framework regions set forth in FIG. 5, 6, 7, 8, 26, or 27, (c) three CDRs of a light chain variable domain provided herein (e.g., SEQ ID NOs:9-11; SEQ ID NOs:25-27; SEQ ID NOs:41-43; or SEQ ID NOs:481-483), and (d) the framework regions of a light chain variable domain set forth in FIG. 2B, 3B, 4B, or 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:536. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:536. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:536.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:536, provided that the heavy chain variable domain includes the amino acid sequences set forth in SEQ ID NOs:529, 530, and 531. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:536, provided that the heavy chain variable domain includes the amino acid sequences set forth in SEQ ID NOs:529, 530, and 531.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having the amino acid sequence set forth in SEQ ID NO:536 or the amino acid set forth in SEQ ID NO:536 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a CD276 polypeptide (e.g., a human CD276 polypeptide) and can include a heavy chain variable domain having the amino acid sequence set forth in SEQ ID NO:536 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:529, 530, and 531.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain comprising (i) a CDR1 that comprises, consists essentially of, or consists of the amino acid sequence set forth in SEQ ID NO:529, (ii) a CDR2 that comprises, consists essentially of, or consists of the amino acid sequence set forth in SEQ ID NO:530, and (iii) a CDR3 that comprises, consists essentially of, or consists of the amino acid sequence set forth in SEQ ID NO:531. As used herein, a “CDR1 that consists essentially of the amino acid sequence set forth in SEQ ID NO:529” is a CDR1 that has zero, one, or two amino acid substitutions within SEQ ID NO:529, that has zero, one, two, three, four, or five amino acid residues directly preceding SEQ ID NO:529, and/or that has zero, one, two, three, four, or five amino acid residues directly following SEQ ID NO:529, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:529 include, without limitation, those set forth in Table 37.

TABLE 37
Exemplary CDR1s that consist essentially of the
amino acid sequence set forth in SEQ ID NO: 529.

| Sequence | SEQ ID NO: |
|---|---|
| SYAMS | 547 |
| SYAMD | 548 |
| SYEMS | 549 |
| SYEMD | 550 |
| SYEMH | 551 |
| TYAMS | 552 |
| TYAMD | 565 |
| TYEMS | 567 |
| SYEMD | 568 |
| TYEMD | 569 |

As used herein, a “CDR2 that consists essentially of the amino acid sequence set forth in SEQ ID NO:530” is a CDR2 that has zero, one, or two amino acid substitutions within SEQ ID NO:530, that has zero, one, two, three, four, or five amino acid residues directly preceding SEQ ID NO:530, and/or that has zero, one, two, three, four, or five amino acid residues directly following SEQ ID NO:530, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:530 include, without limitation, those set forth in Table 38.

TABLE 38
Exemplary CDR2s that consist essentially of the
amino acid sequence set forth in SEQ ID NO: 530.

| Sequence | SEQ ID NO: |
|---|---|
| SIGPRGINTHYADSVK | 570 |
| TIGPKGINTHYADSVK | 571 |
| TIGPRGINSHYADSVK | 572 |
| TIGPRGINTHYAESVK | 573 |
| TIGPRGINTHYADTVK | 574 |
| TIGPRGINTHYADSVR | 575 |
| TIGPRGINTHYADSLK | 576 |
| TLGPRGINTHYADSVK | 577 |
| SIGPRGINTHYADTVK | 578 |
| SIGPRGINTHYADTVR | 579 |

As used herein, a “CDR3 that consists essentially of the amino acid sequence set forth in SEQ ID NO:531” is a CDR3 that has zero, one, or two amino acid substitutions within SEQ ID NO:531, that has zero, one, two, three, four, or five amino acid residues directly preceding SEQ ID NO:531, and/or that has zero, one, two, three, four, or five amino acid residues directly following SEQ ID NO:531, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:531 include, without limitation, those set forth in Table 39.

TABLE 39
Exemplary CDR3s that consist essentially of the
amino acid sequence set forth in SEQ ID NO: 531.

| Sequence | SEQ ID NO: |
|---|---|
| ERVGYNSYGMDV | 580 |
| DKVGYNSYGMDV | 581 |
| DRAGYNSYGMDV | 582 |
| DRVGYQSYGMDV | 583 |
| DRVGYNTYGMDV | 584 |
| DRVGYNSYGMEV | 585 |
| DRVGYNSYGMDL | 586 |
| DKVGYNTYGMDV | 587 |
| DRVGYNTYGMEV | 588 |
| DRVGYNTYGMDA | 589 |

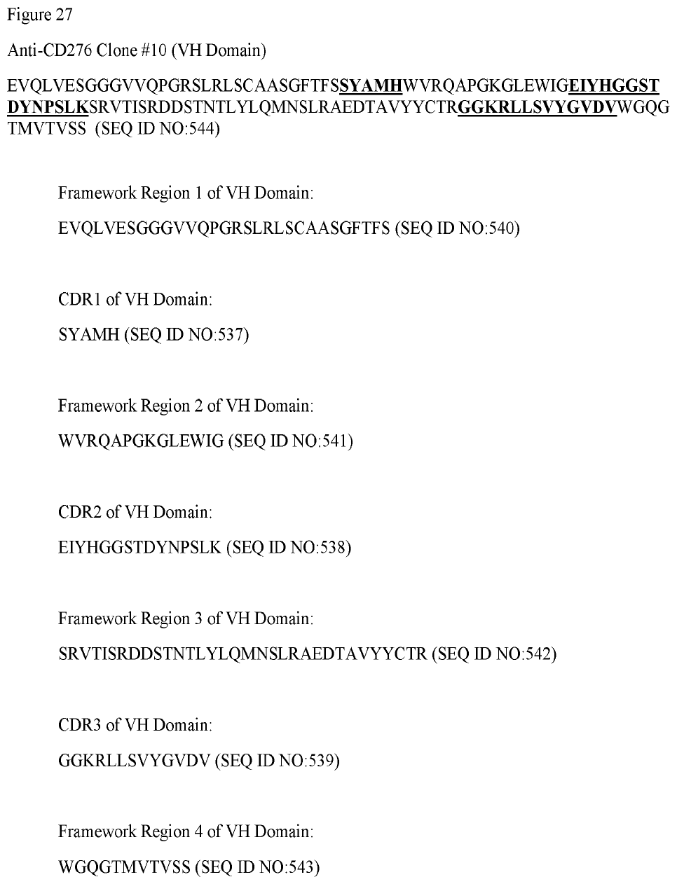

In another embodiment, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having a CDR1 having the amino acid sequence set forth in SEQ ID NO:537 (or a variant of SEQ ID NO:537 with one or two amino acid modifications), a CDR2 having the amino acid sequence set forth in SEQ ID NO:538 (or a variant of SEQ ID NO:538 with one or two amino acid modifications), and a CDR3 having the amino acid sequence set forth in SEQ ID NO:539 (or a variant of SEQ ID NO:539 with one or two amino acid modifications). An example of such an antibody domain having these CDRs and the ability to bind to a CD276 polypeptide (e.g., a human CD276 polypeptide) includes, without limitation, the VH domain set forth in FIG. 27.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) and having a heavy chain variable domain having a CDR1 having the amino acid sequence set forth in SEQ ID NO:537 (or a variant of SEQ ID NO:537 with one or two amino acid modifications), a CDR2 having the amino acid sequence set forth in SEQ ID NO:538 (or a variant of SEQ ID NO:538 with one or two amino acid modifications), and a CDR3 having the amino acid sequence set forth in SEQ ID NO:539 (or a variant of SEQ ID NO:539 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:540 (or a variant of SEQ ID NO:540 with one, two, three, four, five, six, seven, eight, nine, ten, or more amino acid modifications), a framework region 2 having the amino acid sequence set forth in SEQ ID NO:541 (or a variant of SEQ ID NO:541 with one, two, three, four, five, six, seven, eight, nine, ten, or more amino acid modifications), a framework region 3 having the amino acid sequence set forth in SEQ ID NO:542 (or a variant of SEQ ID NO:542 with one, two, three, four, five, six, seven, eight, nine, ten, or more amino acid modifications), and a framework region 4 having the amino acid sequence set forth in SEQ ID NO:543 (or a variant of SEQ ID NO:543 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8 can be designed to include framework regions as set forth in FIG. 27 or can be designed to include one or more framework regions from another antibody or antibody fragment. For example, an antibody domain (e.g., a VH domain) can be designed to include the three CDRs set forth in FIG. 8 and the framework regions set forth in FIG. 27 except that framework region 1 having the amino acid set forth in SEQ ID NO:540 is replaced with a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, or a framework region 1 having the amino acid set forth in SEQ ID NO:532. In another example, an Fab or scFv can be designed to include (a) the three CDRs set forth in FIG. 27, (b) the framework regions set forth in FIG. 5, 6, 7, 8, 26, or 27, (c) three CDRs of a light chain variable domain provided herein (e.g., SEQ ID NOs:9-11; SEQ ID NOs:25-27; SEQ ID NOs:41-43; or SEQ ID NOs:481-483), and (d) the framework regions of a light chain variable domain set forth in FIG. 2B, 3B, 4B, or 18B.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:544. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:544. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:544.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain that includes an amino acid sequence having at least 90 percent identity to the amino acid sequence set forth in SEQ ID NO:544, provided that the heavy chain variable domain includes the amino acid sequences set forth in SEQ ID NOs:537, 538, and 539. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:544, provided that the heavy chain variable domain includes the amino acid sequences set forth in SEQ ID NOs:537, 538, and 539.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain having the amino acid sequence set forth in SEQ ID NO:544 or the amino acid set forth in SEQ ID NO:544 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a CD276 polypeptide (e.g., a human CD276 polypeptide) and can include a heavy chain variable domain having the amino acid sequence set forth in SEQ ID NO:544 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:537, 538, and 539.

In some cases, a binder (e.g., an antibody, antigen binding fragment, antibody domain, a CAR, a cell engager, and/or an ADC) provided herein having the ability to bind to a CD276 polypeptide (e.g., a human CD276 polypeptide) can include a heavy chain variable domain comprising (i) a CDR1 that comprises, consists essentially of, or consists of the amino acid sequence set forth in SEQ ID NO:537, (ii) a CDR2 that comprises, consists essentially of, or consists of the amino acid sequence set forth in SEQ ID NO:538, and (iii) a CDR3 that comprises, consists essentially of, or consists of the amino acid sequence set forth in SEQ ID NO:539. As used herein, a “CDR1 that consists essentially of the amino acid sequence set forth in SEQ ID NO:537” is a CDR1 that has zero, one, or two amino acid substitutions within SEQ ID NO:537, that has zero, one, two, three, four, or five amino acid residues directly preceding SEQ ID NO:537, and/or that has zero, one, two, three, four, or five amino acid residues directly following SEQ ID NO:537, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR1 that consists essentially of the amino acid sequence set forth in SEQ ID NO:537 include, without limitation, those set forth in Table 40.

TABLE 40
Exemplary CDR1s that consist essentially of the
amino acid sequence set forth in SEQ ID NO: 537.

| Sequence | SEQ ID NO: |
|---|---|
| SYAMS | 590 |
| SYAMD | 591 |
| SYEMS | 592 |
| SYEMD | 593 |
| SYEMH | 594 |
| TYAMS | 595 |
| TYAMD | 596 |
| TYEMS | 597 |
| SYEMD | 598 |
| TYEMD | 599 |

As used herein, a “CDR2 that consists essentially of the amino acid sequence set forth in SEQ ID NO: 538” is a CDR2 that has zero, one, or two amino acid substitutions within SEQ TD NO:538, that has zero, one, two, three, four, or five amino acid residues directly preceding SEQ TD NO:538, and/or that has zero, one, two, three, four, or five amino acid residues directly following SEQ ID NO:538, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR2 that consists essentially of the amino acid sequence set forth in SEQ ID NO:538 include, without limitation, those set forth in Table 41.

TABLE 41
Exemplary CDR2s that consist essentially of the
amino acid sequence set forth in SEQ ID NO: 538.

| Sequence | SEQ ID NO: |
|---|---|
| DIYHGGSTDYNPSLK | 600 |
| ELYHGGSTDYNPSLK | 601 |
| EIYDGGSTDYNPSLK | 602 |
| EIYHGGTTDYNPSLK | 603 |
| EIYHGGSSDYNPSLK | 604 |
| EIYHGGSTEYNPSLK | 605 |
| EIYHGGSTDYQPSLK | 606 |
| EIYHGGSTDYNPTLK | 607 |
| EIYHGGSTDYNPSIK | 608 |
| EIYHGGSTDYNPSLR | 609 |

As used herein, a “CDR3 that consists essentially of the amino acid sequence set forth in SEQ ID NO:539” is a CDR3 that has zero, one, or two amino acid substitutions within SEQ TD NO:539, that has zero, one, two, three, four, or five amino acid residues directly preceding SEQ TD NO:539, and/or that has zero, one, two, three, four, or five amino acid residues directly following SEQ ID NO:539, provided that the binder (e.g., an antibody, antigen binding fragment, antibody domain, a CAR, a cell engager, and/or an ADC) maintains its basic ability to bind to a CD276 polypeptide (e.g., a human CD276 polypeptide). Examples of a CDR3 that consists essentially of the amino acid sequence set forth in SEQ ID NO:539 include, without limitation, those set forth in Table 42.

TABLE 42
Exemplary CDR3s that consist essentially of the
amino acid sequence set forth in SEQ ID NO: 539.

| Sequence | SEQ ID NO: |
|---|---|
| GGRRLLSVYGVDV | 610 |
| GGKKLLSVYGVDV | 611 |
| GGRKLLSVYGVDV | 612 |
| GGKRLLTVYGVDV | 613 |
| GGKRLLSVYGVEV | 614 |
| GGKRLLSVYGVDL | 615 |
| GGKRLLSVYGLDV | 616 |
| GGKRLLTVYGVEV | 617 |
| GGKKLLSVYGVEV | 618 |
| GGRRLLSVYGVEV | 619 |

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a CD276 polypeptide (e.g., a human CD276 polypeptide) are further described in Table 43.

TABLE 43
Representative number of binders.

| | | SEQ ID NOs of | | | SEQ ID NOs of | |
|---|---|---|---|---|---|---|
| | SEQ ID NOs of | Heavy Chain | | SEQ ID NOs of | Light Chain | |
| | Heavy Chain | Variable | SEQ ID NO of | Light Chain | Variable | SEQ ID NO of |
| Clone # | Variable | Domain/Region | Heavy Chain | Variable | Domain/Region | Light Chain |
| (Antibody | Domain/Region | Framework | Variable | Domain/Region | Framework | Variable |
| type) | CDRs | Regions | Domain/Region | CDRs | Regions | Domain/Region |
| #1 (Fab) | 1, 2, 3 | 4, 5, 6, 7 | 8 | 9, AAS, 11 | 12, 13, 14, 15 | 16 |
| #2 (Fab) | 17, 18, 19 | 20, 21, 22, 23 | 24 | 25, AAS, 27 | 28, 29, 30, 31 | 32 |
| #3 (Fab) | 33, 34, 35 | 36, 37, 38, 39 | 40 | 41, AAS, 43 | 44, 45, 46, 47 | 48 |
| #4 (VH | 49, 50, IGY | 52, 53, 54, 55 | 56 | | | |
| domain) | | | | | | |
| #5 (VH | 57, 58, 59 | 60, 61, 62, 63 | 64 | | | |
| domain) | | | | | | |
| #6 (VH | 65, 66, 67 | 68, 69, 70, 71 | 72 | | | |
| domain) | | | | | | |
| #7 (VH | 73, 74, IDY | 76, 77, 78, 79 | 80 | | | |
| domain) | | | | | | |
| #8 (Fab) | 473, 474, 475 | 476, 477, 478, | 480 | 481, AAS, 483 | 484, 485, 486, | 488 |
| | | 479 | | | 487 | |
| #9 (VH | 529, 530, 531 | 532, 533, 534, | 536 | | | |
| domain) | | 535 | | | | |
| #10 (VH | 537, 538, 539 | 540, 541, 542, | 544 | | | |
| domain) | | 543 | | | | |

Table 44 includes an alternative designation that can be used to refer to each of Clones #1-#10.

TABLE 44
Alternative nomenclature for Clones #1-#10.

| Clone # | Alternative names |
|---|---|
| 1 | ab1 |
| 2 | ab2 |
| 3 | ab3 |
| 4 | ab4 |
| 5 | ab5 |
| 6 | ab6 |
| 7 | ab7 |
| 8 | ab8 |
| 9 | ab9; A10 |
| 10 | ab10; C7 |

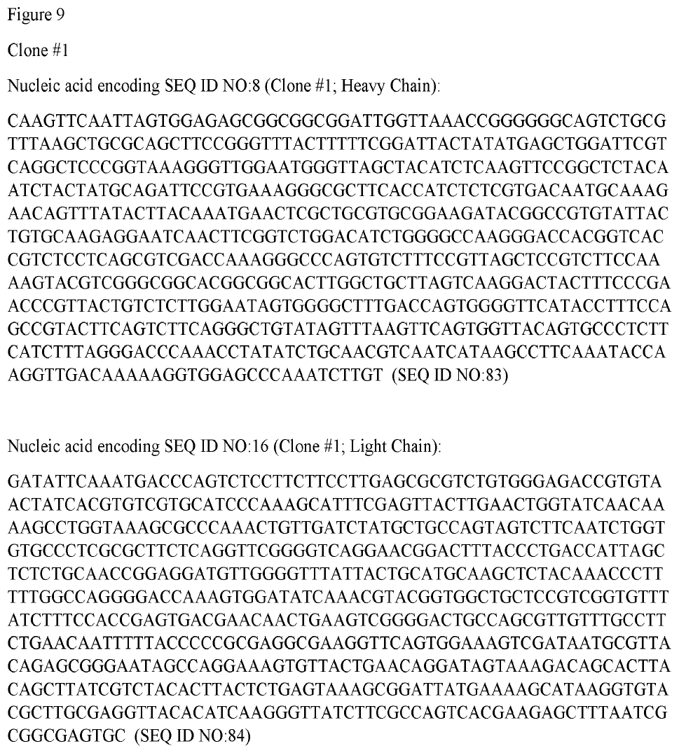

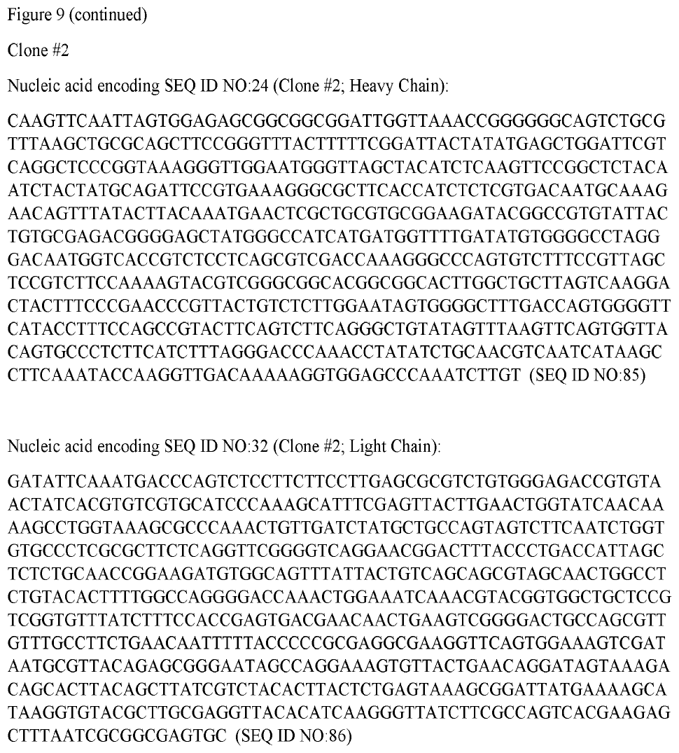

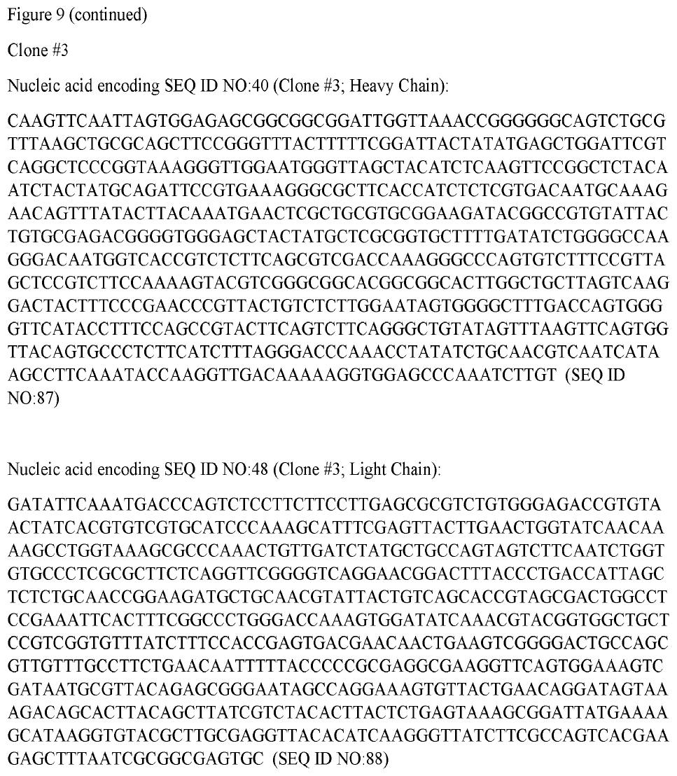

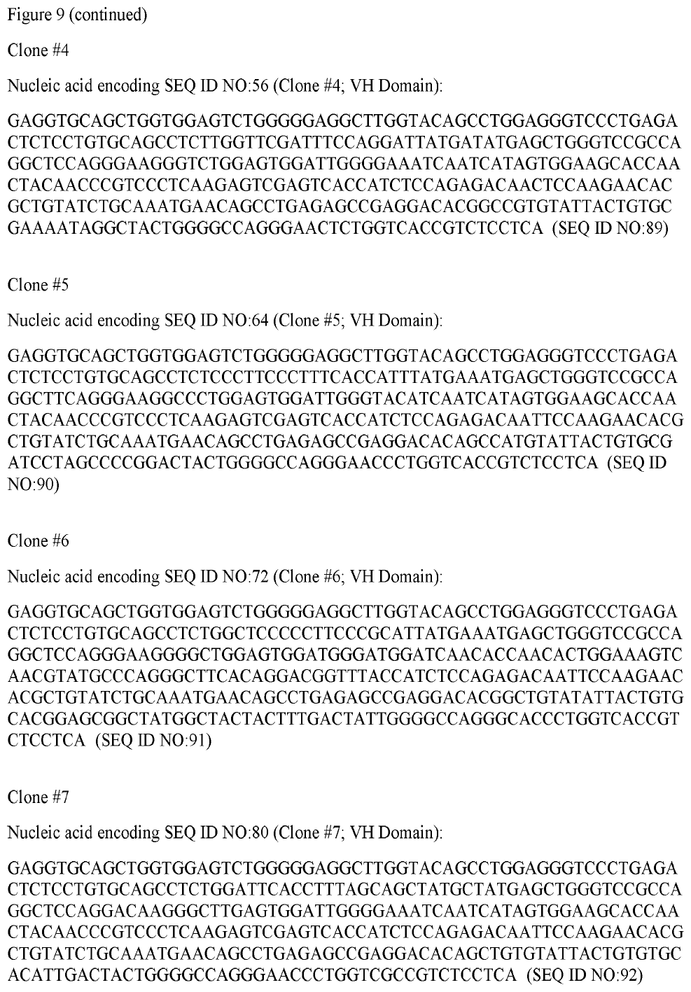

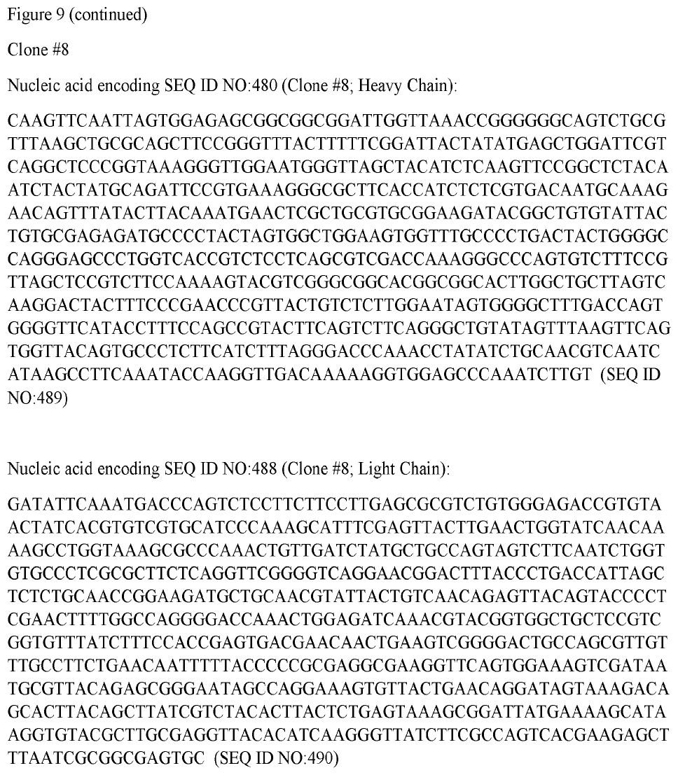

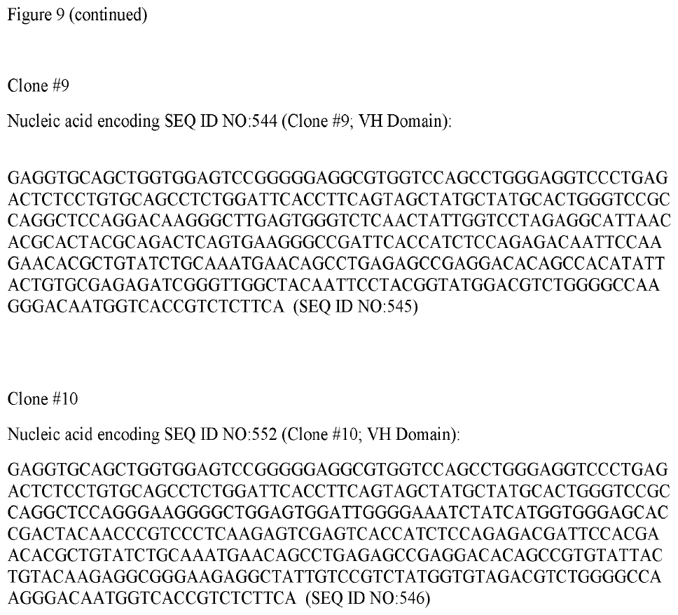

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. FIG. 9 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a CD276 polypeptide (e.g., a human CD276 polypeptide). In some cases, a bispecific binder provided herein can be designed to bind to two different epitopes of the same CD276 polypeptide (e.g., a human CD276 polypeptide). In some cases, a bispecific binder provided herein can bind to a CD276 polypeptide (e.g., a human CD276 polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a CD276 polypeptide (e.g., a human CD276 polypeptide) and (b) at least one drug covalently linked directly or indirectly to that antigen binding domain. Any appropriate binder (e.g., an antibody, antigen binding fragment, and/or antibody domain) provided herein and having the ability to bind to a CD276 polypeptide (e.g., a human CD276 polypeptide) can be used as an antigen binding domain to make an ADC described herein. For example, any of the binders set forth in Table 43 can be used to make an ADC having the ability to bind to a CD276 polypeptide (e.g., a human CD276 polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a CD276 polypeptide (e.g., a human CD276 polypeptide) to form an ADC provided herein. For example, cleavable or non-cleavable ADC linkers can be used to covalently attach one or more drugs to an antigen binding domain having the ability to bind to a CD276 polypeptide (e.g., a human CD276 polypeptide) to form an ADC provided herein. Examples of ADC linkers can be used to covalently attach one or more drugs to an antigen binding domain having the ability to bind to a CD276 polypeptide (e.g., a human CD276 polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in FIG. 2A, 3A, 4A, or 18A or the VH domain set forth in FIG. 5, 6, 7, 8, 26, or 27. In another example, an isolated nucleic acid molecule provided herein can include a nucleic acid sequence encoding a light chain variable domain such as a light chain variable domain as set forth in FIG. 2B, 3B, 4B, or 18B. In some cases, an isolated nucleic acid molecule provided herein can include a nucleic acid sequence encoding both (a) a heavy chain variable domain and (b) a light chain variable domain, with or without, encoding a linker polypeptide set forth in FIG. 10. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors) and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5α, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukayotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a CD276 polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a CD276 polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a CD276 polypeptide (e.g., a human CD276 polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a CD276 polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a CD276 polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, liver cancer, colorectal cancer, brain cancer, skin cancer (e.g., melanoma), lung cancer, prostate cancer, breast cancer (e.g., PR positive breast cancer, ER positive breast cancer, HER2 positive breast cancer, or triple negative breast cancer), ovarian cancer, cervical cancer, esophageal cancer, glioma, kidney cancer, mesothelioma, and pancreatic cancer. In some cases, a solid cancer such as liver cancer, colorectal cancer, brain cancer, skin cancer (e.g., melanoma), lung cancer, prostate cancer, breast cancer (e.g., PR positive breast cancer, ER positive breast cancer, HER2 positive breast cancer, or triple negative breast cancer), ovarian cancer, cervical cancer, esophageal cancer, glioma, kidney cancer, mesothelioma, or pancreatic cancer can be treated as described herein. In some cases, cancer such as lymphoma (e.g., B cell lymphomas such as diffuse large cell lymphoma (DLBCL)), leukemia (e.g., chronic lymphocytic leukemia (CLL) and acute lymphoblastic leukemia (ALL)), or acute myeloid leukemia can be treated as described herein. In some cases, a mammal (e.g., a human) having a CD276⁺ cancer (e.g., a CD276⁺ liver cancer, CD276⁺ colorectal cancer, CD276⁺ brain cancer, CD276⁺ skin cancer (e.g., CD276⁺ melanoma), CD276⁺ lung cancer, CD276⁺ prostate cancer, CD276⁺ breast cancer (e.g., CD276⁺ PR positive breast cancer, CD276⁺ ER positive breast cancer, CD276⁺ HER2 positive breast cancer, or CD276⁺ triple negative breast cancer), CD276⁺ ovarian cancer, CD276⁺ cervical cancer, CD276⁺ esophageal cancer, CD276⁺ glioma, CD276⁺ kidney cancer, CD276⁺ mesothelioma, or CD276⁺ pancreatic cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a CD276 polypeptide (e.g., a human CD276 polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a CD276 polypeptide (e.g., a human CD276 polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a CD276 polypeptide (e.g., a human CD276 polypeptide). In some cases, a binder (e.g., an antibody, antigen binding fragment, and/or antibody domain) provided herein can be immobilized, e.g., on a support, and retention of a CD276 polypeptide (e.g., a human CD276 polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a CD276 polypeptide (e.g., a human CD276 polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ⁹⁹mTc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human CD276 Polypeptide

Large phage displayed antibody fragments and antibody domain libraries were panned and screened to identify monoclonal antigen binding fragments and monoclonal antibody domains that bind to a human CD276 polypeptide. To identify such monoclonal antibodies, monoclonal antigen binding fragments, and monoclonal antibody domains, a human CD276 polypeptide set forth in FIG. 1 was fused to the Fc region of human IgG1 at the C-terminus of the CD276 sequence, and the CD276-Fc polypeptide was used for panning of human Fab and VH domain phage-displayed libraries. Three Fab antibody fragments (Clones: #1, #2, and #3; FIGS. 2-4) and four VH domains (Clones: #4, #5, #6, and #7; FIGS. 5-8) were identified. In another panning, the amino acid sequence of SEQ ID NO:471 (a fragment of a human CD276 polypeptide containing a C2 domain) was fused to a 6×HIS tag for panning of a human Fab domain phage-displayed library. One Fab antibody fragment (Clone #8; FIG. 18) was identified. In another panning, another human VH domain phage-displayed library was used to pan against the same CD276 polypeptide. Two additional VH domains (Clones #9 and #10; FIGS. 26 and 27) were identified.

Binding affinity and specificity to a human CD276 polypeptide were tested using an ELISA and Omburtamab (8H9) as a competitor. Clones #1-#8 exhibited high affinity binding having EC₅₀ values of 34 nM, 46 nM, 26 nM, 0.9 nM, 1.2 nM, 10 nM, 0.6 nM, and 4 nM, respectively. Clones #1 and #4-#7 also competed with Omburtamab (8H9) for binding to CD276 expressed by Du145 and PC3 cells, demonstrating that they can bind to CD276 present on cells and prevent the interaction of an anti-CD276 antibody with CD276 present on cells. Clones #2 and #3 did not compete with Omburtamab for binding to CD276 present on these cells, but they did bind to the CD276 expressing Du145 and PC3 cells. Clone #8 competed with DS-5573a (a full-sized anti-human CD276 afucosylated humanized antibody (150 kDa); Daiichi Sankyo) for binding to CD276 expressed on cells, demonstrating that it can bind to CD276 present on cells and prevent the interaction of an anti-CD276 antibody with CD276 present on cells. Clones #1-#7 did not compete with DS-5573a for binding to CD276 present on cells.

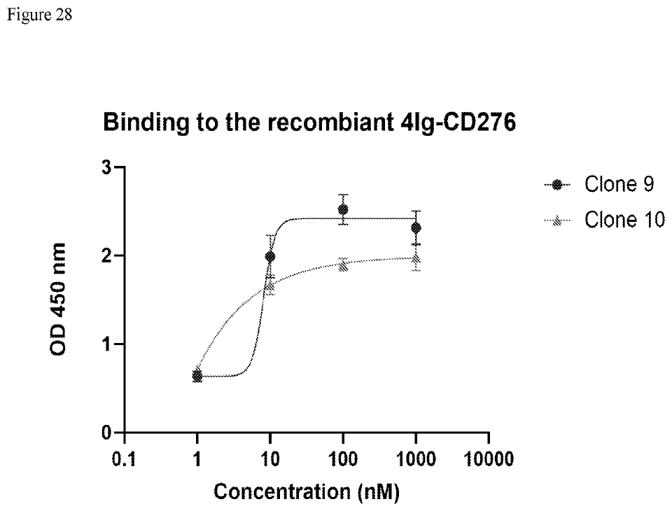

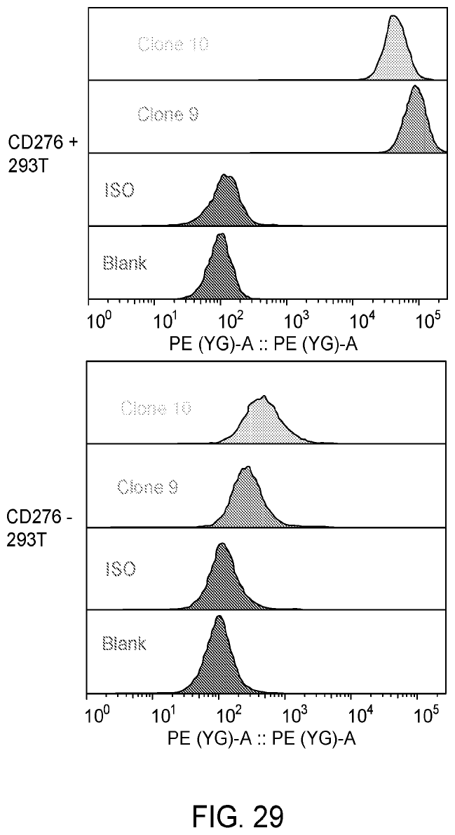

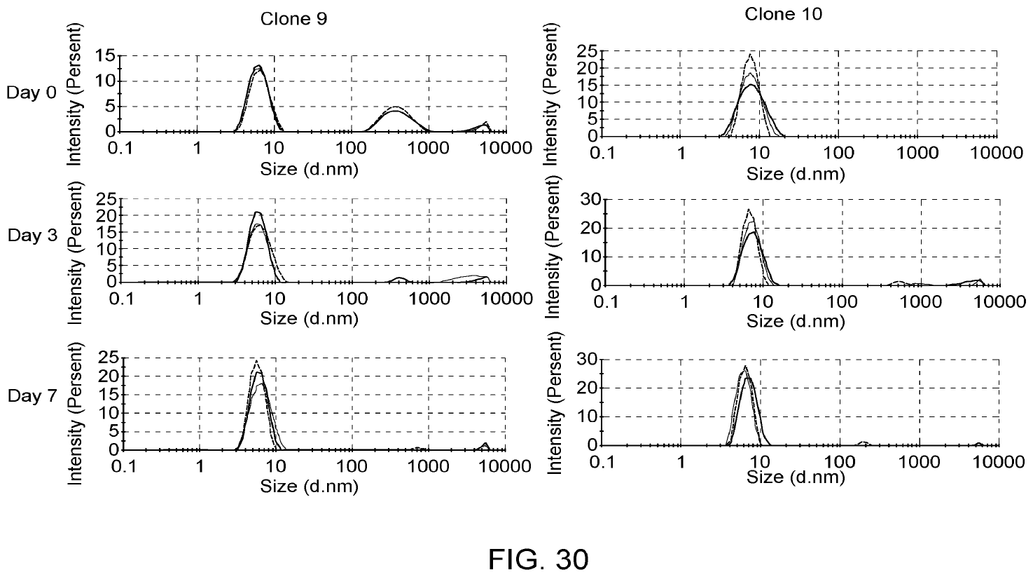

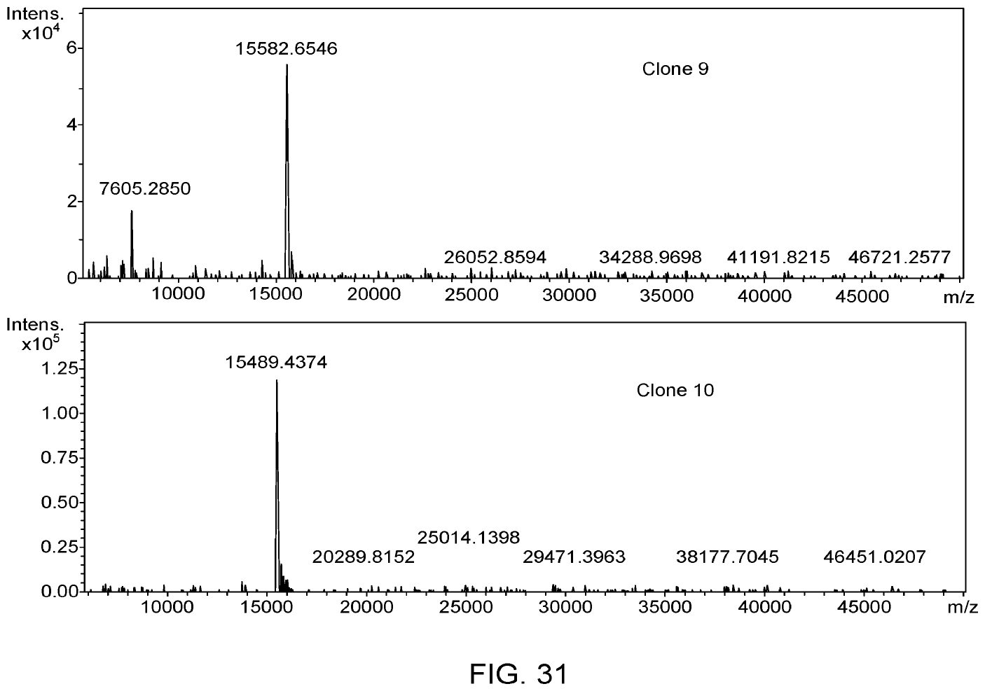

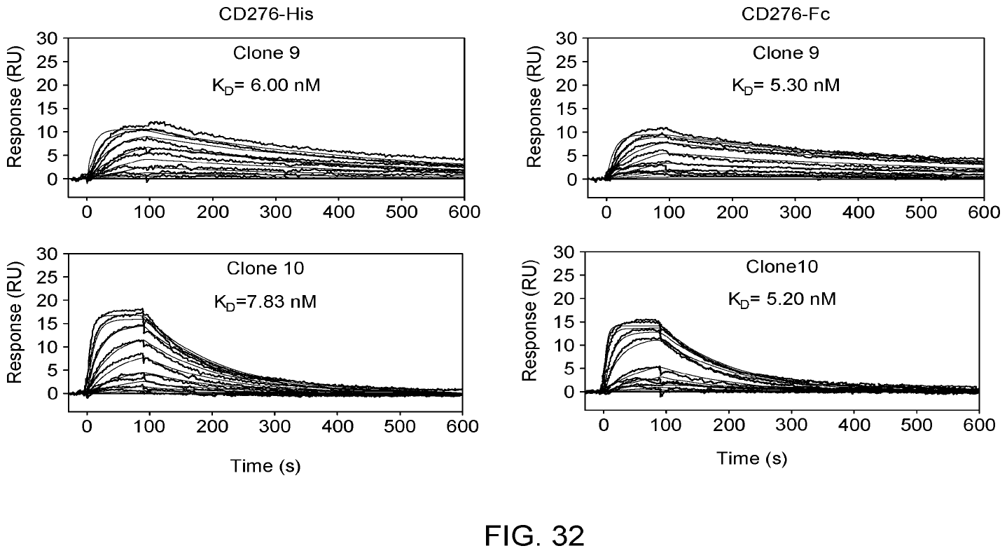

Clones #9 and #10, two VH binders, bound to a CD276 polypeptide with EC₅₀ values of 8.1 and 1.2 nM, respectively, as tested by ELISA. They were able bind to cell surface associated CD276 polypeptides, with no binding to cells that did not express CD276 polypeptides. Clone #9 and #10 exhibited aggregation resistance as tested by dynamic light scattering. The molecular weight of these two clones were validated by mass spectrum. The binding kinetics of clone #9 and #10 to the 4Ig CD276 polypeptide with both his tag and Fc tag were measured by surface plasmon resonance. The results consistently showed binding dissociation constants (KD) of single digit nM, which was also consistent with the EC₅₀ values exhibited via ELISA. See, FIGS. 28-32.

### Example 2—Designing CARs from Binders Having the Ability to Bind to a Human CD276 Polypeptide

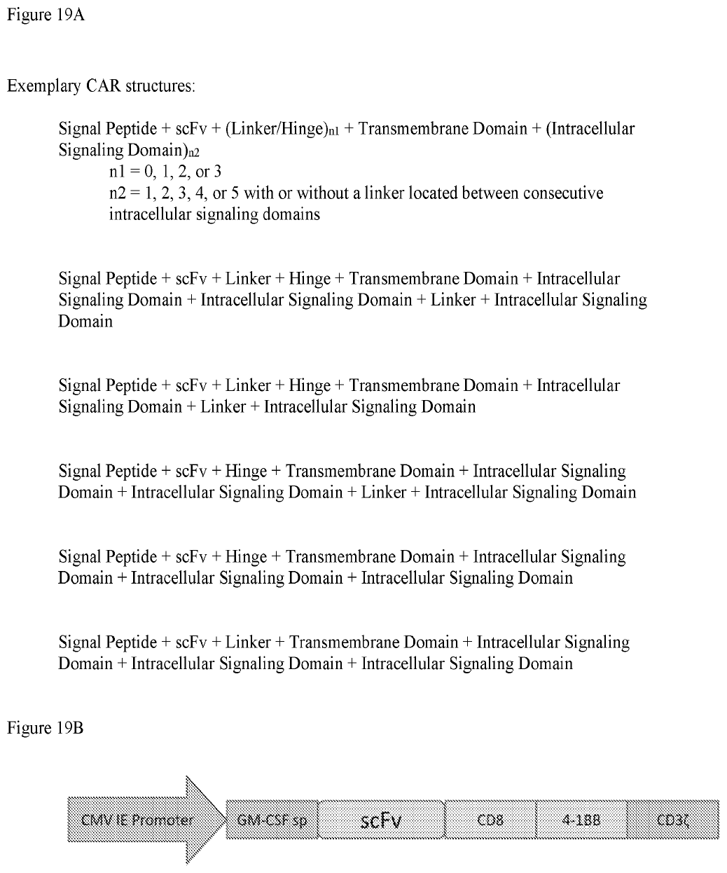

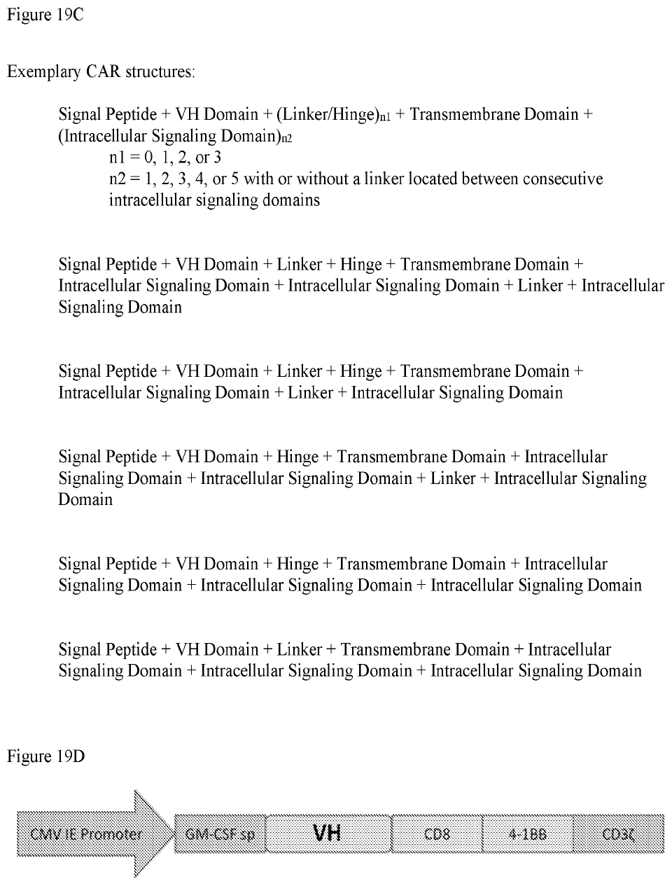

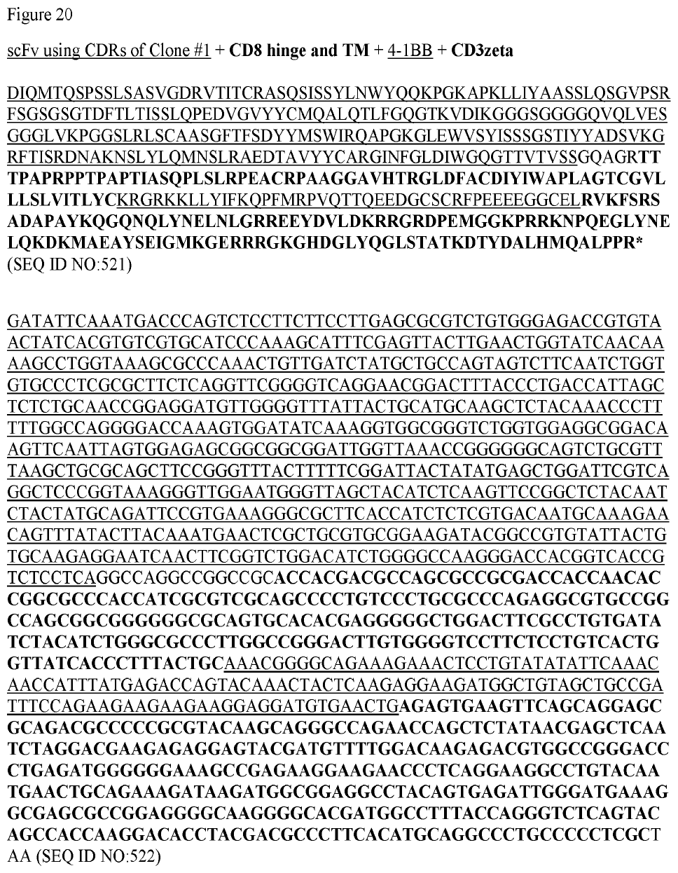

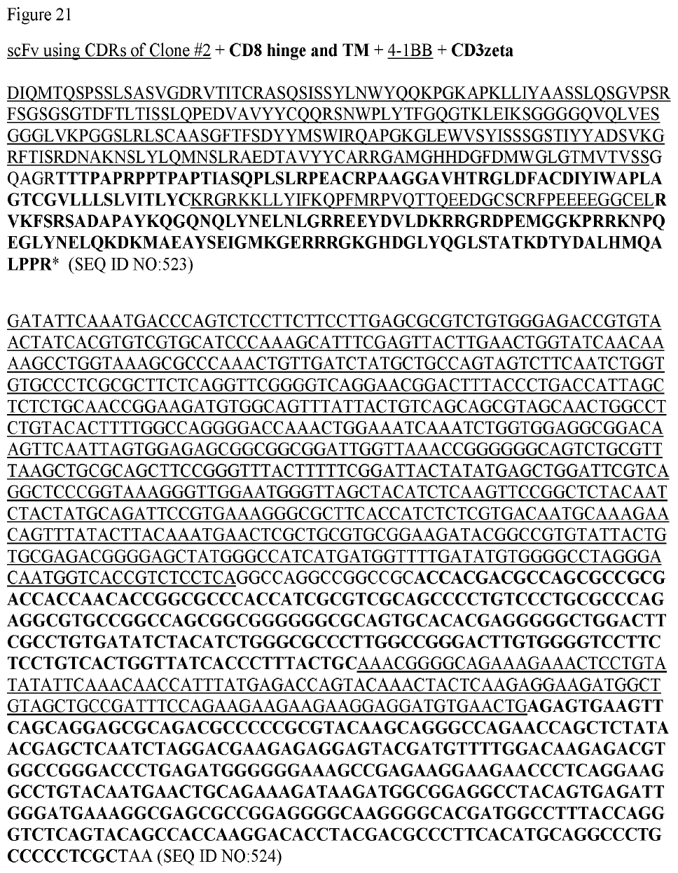

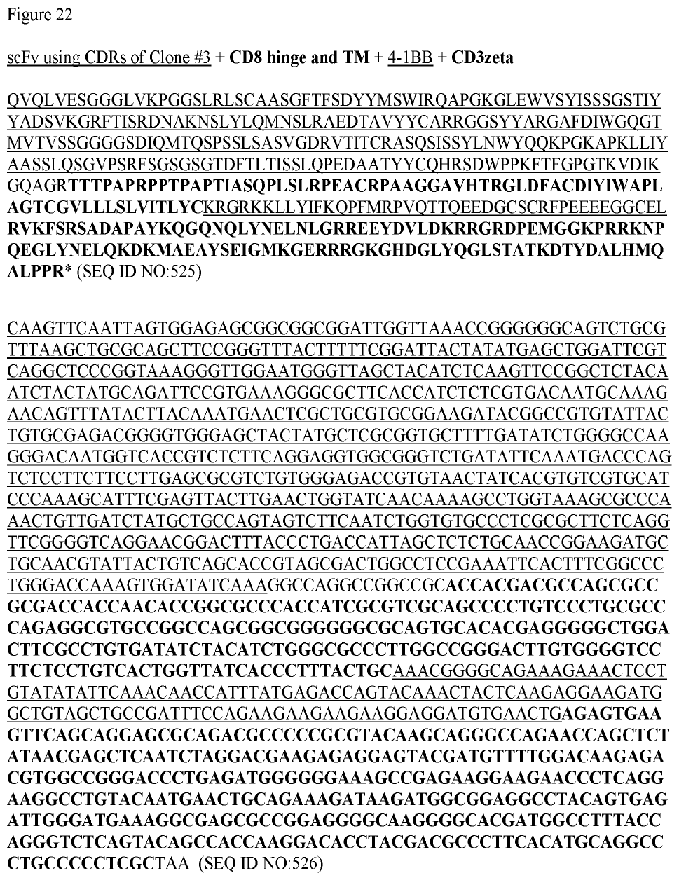

The heavy and light chains of the Fab's of Clones #1-3 were used to create scFv's, and those scFv's were used to create vectors designed to express CARs having the ability to bind to CD276 polypeptides. See, e.g., FIGS. 19A-D. The amino acid sequence of CAR #1, CAR #2, and CAR #3 and the nucleic acid sequences encoding those CARs are set forth in FIGS. 20, 21, and 22, respectively. The vectors encoding CAR #1, CAR #2, or CAR #3 were transfected into T cells that were then used in a killing assay as effector cells. Briefly, 10,000 CAR-T cells were incubated with 100,000 target cells (CHO-K1-CD276-Luci, Du145 cancer cells, or PC3 cancer cells; E:T=10:1) per well in 96 well plates for 24 hours. Untransduced human T cells were used as a negative control. A maximum LDH release control was used where 2 μL of 10% Triton X-100 per 100 μL was added to target cell only wells for 10-15 minutes before collecting the samples for LDH detection. LDH release was detected using LDH-Glo™ Cytotoxicity Assay kit (Promega J2380) following the manufacturer's instructions. The percent cytotoxicity was calculated using the following formula: % Lysis=100× (Experimental LDH Release−Medium Background)/(Maximum LDH Release Control−Medium Background).

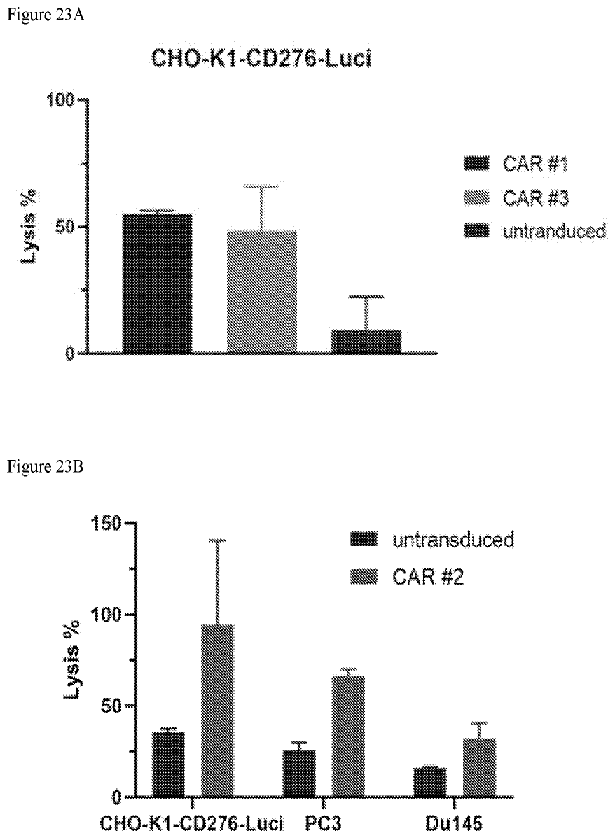

CHO-K1-CD276-Luci cell lysis was observed for T cells expressing CAR #1 and T cells expressing CAR #3 (FIG. 23A). CHO-K1-CD276-Luci cell, PC3 cancer cell, and Du145 cancer cell lysis was observed for T cells expressing CAR #2 (FIG. 23B). These results demonstrate that CARs having the ability to bind to CD276 polypeptides can be designed using the CDRs and/or binders provided herein.

### Example 3—Designing BiTE's from Binders Having the Ability to Bind to a Human CD276 Polypeptide

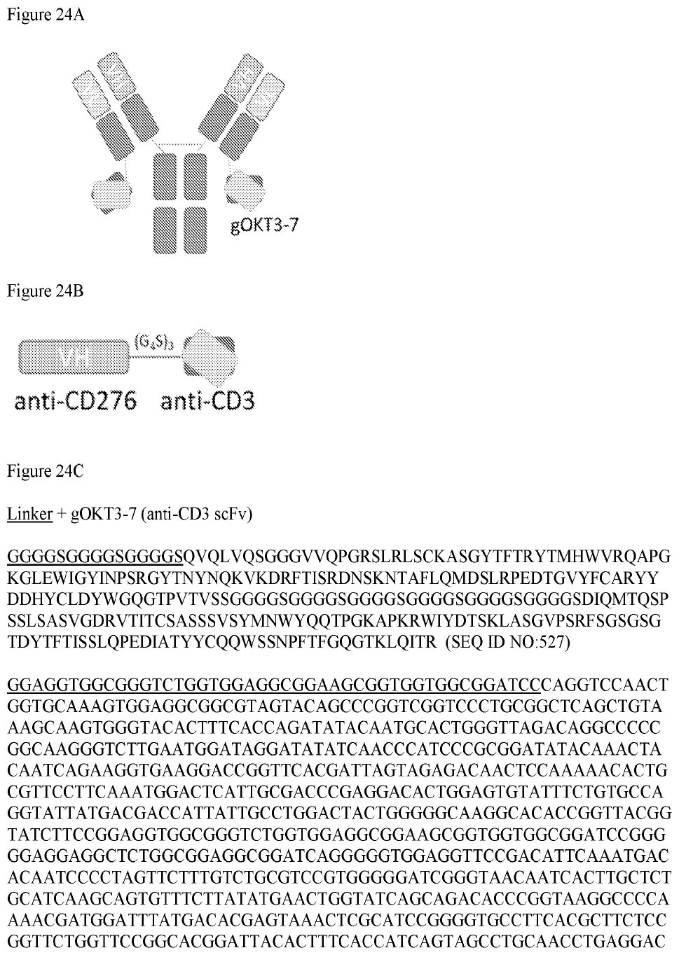

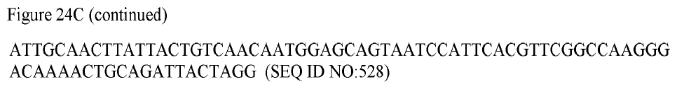

The heavy and light variable domains of the Fab's of Clones #1-3 along with an anti-CD3 scFv (gOKT3-7) were used to create vectors designed to express BiTEs (BiTE #1, BiTE #2, and BiTE #3) having the ability to bind to CD276 polypeptides and CD3 polypeptides. See, e.g., FIG. 24A. The Ig portion of the molecules was based on IgG1, and the amino acid and nucleic acid sequences for the linker and anti-CD3 scFv are set forth in FIG. 24C. In addition, the VH domain of Clone #5 along with an anti-CD3 scFv (gOKT3-7) were used to create a vector designed to express a BiTE (BiTE #5) having the ability to bind to CD276 polypeptides and CD3 polypeptides. See, e.g., FIG. 24B. A control BiTE (Control BiTE) was designed to include an antibody that does not bind to CD276.

The isolated BiTE's were tested in a killing assay that included target cells and T cells as effector cells. Briefly, 10,000 target cells (CHO-K1-CD276-Luci) were incubated with serially diluted BiTE's (BiTE #1, BiTE #2, BiTE #3, BiTE #5, or Control BiTE), followed by the addition of activated human T cells (E:T=20:1) per well in 96 well plates for 24 hours. A maximum protease activity control was used where 2 μL of 10% Triton X-100 per 100 μL was added to target cell only wells for 10-15 minutes before collecting the samples. Extracellular activity of dead-cell protease was detected using the Bio-Glo™ Luciferase Assay System (Promega G7941) or CytoTox-Glo™ Cytotoxicity Assay kit (Promega G9290) following the manufacturer's instructions. The percent cytotoxicity was calculated using the following formula: % Lysis=100×(Experimental signal−Medium Background)/(Maximum signal−Medium Background).

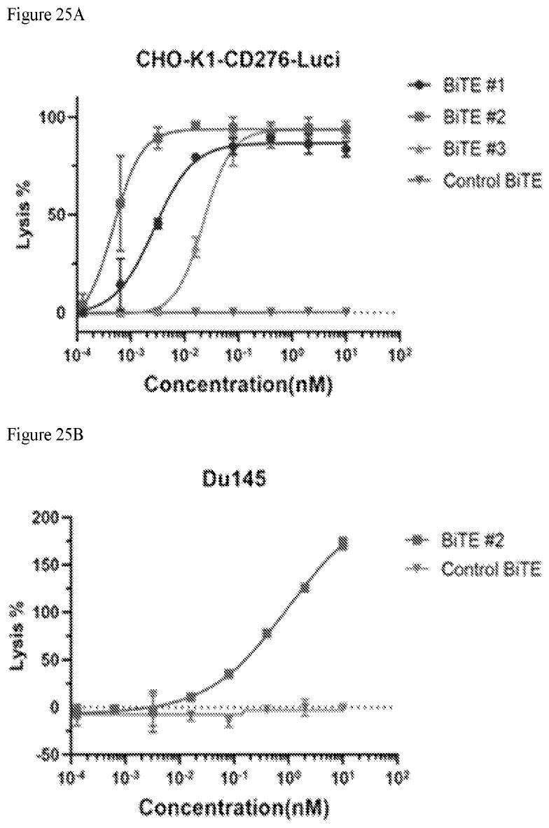

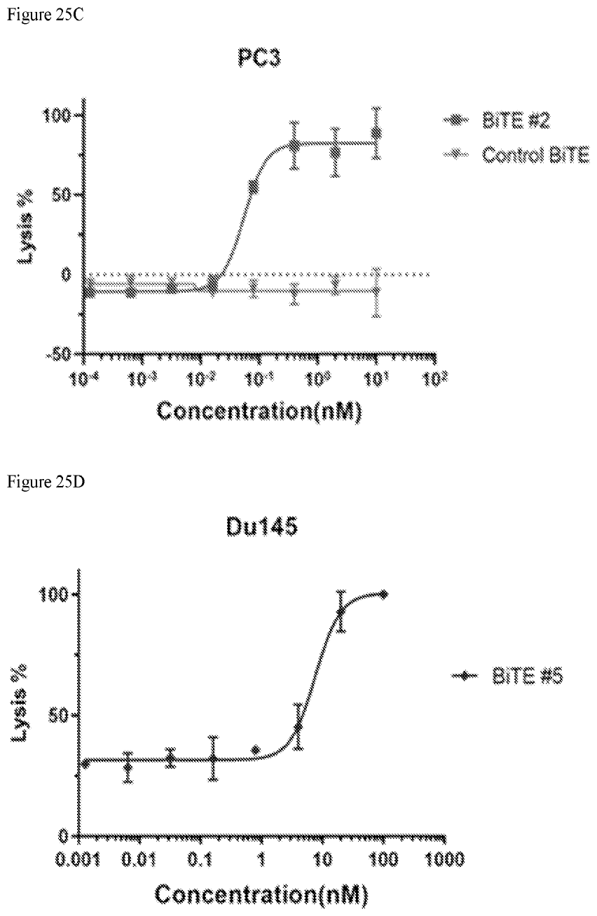

CHO-K1-CD276-Luci cell lysis was observed for T cells incubated in the presence of BiTE #1, BiTE #2, or BiTE #3, but not Control BiTE (FIG. 25A). BiTE #2 also was tested using Du145 cancer cells and PC3 cancer cells as target cells. In both cases, cell lysis was observed for T cells incubated in the presence of BiTE #2, but not Control BiTE (FIGS. 25B and 25C). Likewise, Du145 cancer cell lysis was observed for T cells incubated in the presence of BiTE #5 (FIG. 25D).

These results demonstrate that cell engagers (e.g., BiTEs) having the ability to bind to CD276 polypeptides can be designed using the CDRs and/or binders provided herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

2-22. (canceled)

23. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

24-48. (canceled)

49. An antibody domain comprising:
(i) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(ii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(iii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(iv) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions).
(v) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(vi) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

50-60. (canceled)

61. A chimeric antigen receptor comprising an antigen binding domain, a hinge, a transmembrane domain, and one or more signaling domains, wherein said antigen binding domain comprises an antibody, an antigen-binding fragment, or an antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

62. The chimeric antigen receptor of claim 61, wherein said antigen binding domain comprises a scFv having the ability to bind to a CD276 polypeptide.

63-66. (canceled)

67. A cell comprising a chimeric antigen receptor of claim 61.

68. The cell of claim 67, wherein said cell is a T cell, a stem cell, or an NK cell.

69. A cell engager comprising a first antigen binding domain, a linker, and a second antigen binding domain, wherein said first antigen binding domain comprises an antibody, an antigen-binding fragment, or an antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

70-82. (canceled)

83. A nucleic acid comprising a nucleic acid sequence encoding at least part of an antibody, an antigen-binding fragment, or an antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

84-87. (canceled)

88. A nucleic acid comprising a nucleic acid sequence encoding a chimeric antigen receptor or a cell engager comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

89-93. (canceled)

94. An antibody-drug conjugate (ADC) comprising an antigen binding domain covalently linked to a drug, wherein said antigen binding domain comprises an antibody, an antigen binding fragment, or an antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

95-97. (canceled)

98. A composition comprising an antibody, an antigen binding fragment, or an antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

99-101. (canceled)

102. A composition comprising a cell engager, a cell comprising a chimeric antigen receptor, or an ADC, wherein said cell engager, said chimeric antigen receptor, and said ADC comprises:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

103-104. (canceled)

105. The composition of claim 102, wherein said composition comprises a checkpoint inhibitor.

106. The composition of claim 105, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

107. A method of treating a mammal having cancer, wherein said method comprises administering, to said mammal, a composition of claim 98 or claim 102.

108-111. (canceled)

112. A method of treating a mammal having cancer, wherein said method comprises:
(a) administering, to said mammal, said composition of claim 98 or claim 102, and
(b) administering, to said mammal, a composition comprising a checkpoint inhibitor.

113-117. (canceled)

118. A method for binding a binding molecule to a CD276 polypeptide, wherein said method comprises contacting said CD276 polypeptide with an antibody, an antigen binding fragment, or an antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

119-122. (canceled)

123. A method for binding a binding molecule to a CD276 polypeptide, wherein said method comprises contacting said CD276 polypeptide with a chimeric antigen receptor, a cell engager, or an ADC comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions), and a light chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iv) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(v) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vi) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(viii) a heavy chain variable domain or region comprising the amino acid sequences set forth in SEQ ID NO:473 (or SEQ ID NO:473 with one, two, or three amino acid additions, deletions, or substitutions), SEQ ID NO:474 (or SEQ ID NO:474 with one, two, or three amino acid additions, deletions, or substitutions), and SEQ ID NO:475 (or SEQ ID NO:475 with one, two, or three amino acid additions, deletions, or substitutions), and a light chain variable domain or region comprising the amino acid sequences set forth in SEQ ID NO:481 (or SEQ ID NO:481 with one, two, or three amino acid additions, deletions, or substitutions), SEQ ID NO:482 (or SEQ ID NO:482 with one, two, or three amino acid additions, deletions, or substitutions), and SEQ ID NO:483 (or SEQ ID NO:483 with one, two, or three amino acid additions, deletions, or substitutions);
(ix) a heavy chain variable domain or region comprising the amino acid sequences set forth in SEQ ID NO:529 (or SEQ ID NO:529 with one, two, or three amino acid additions, deletions, or substitutions), SEQ ID NO:530 (or SEQ ID NO:530 with one, two, or three amino acid additions, deletions, or substitutions), and SEQ ID NO:531 (or SEQ ID NO:531 with one, two, or three amino acid additions, deletions, or substitutions); or
(x) a heavy chain variable domain or region comprising the amino acid sequences set forth in SEQ ID NO:537 (or SEQ ID NO:537 with one, two, or three amino acid additions, deletions, or substitutions), SEQ ID NO:538 (or SEQ ID NO:538 with one, two, or three amino acid additions, deletions, or substitutions), and SEQ ID NO:539 (or SEQ ID NO:539 with one, two, or three amino acid additions, deletions, or substitutions).

124. (canceled)

125. The method of claim 123, wherein said contacting is performed in vivo.

126-127. (canceled)


SEQUENCE LISTING (619 sequences)

SEQ ID NO: 1 - protein, 8 aa, synthetic construct
GFTFSDYY

SEQ ID NO: 2 - protein, 8 aa, synthetic construct
ISSSGSTI

SEQ ID NO: 3 - protein, 10 aa, synthetic construct
ARGINFGLDI

SEQ ID NO: 4 - protein, 25 aa, synthetic construct
QVQLVESGGGLVKPGGSLRLSCAAS

SEQ ID NO: 5 - protein, 17 aa, synthetic construct
MSWIRQAPGKGLEWVSY

SEQ ID NO: 6 - protein, 38 aa, synthetic construct
YYADSVKGRFTISRDNAKNSLYLQMNSLRAEDTAVYYC

SEQ ID NO: 7 - protein, 11 aa, synthetic construct
WGQGTTVTVSS

SEQ ID NO: 8 - protein, 220 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGINFGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 6 aa, synthetic construct
QSISSY

SEQ ID NO: 10 - skipped

SEQ ID NO: 11 - protein, 7 aa, synthetic construct
MQALQTL

SEQ ID NO: 12 - protein, 26 aa, synthetic construct [sequence omitted]

SEQ ID NO: 13 - protein, 17 aa, synthetic construct
LNWYQQKPGKAPKLLIY

SEQ ID NO: 14 - protein, 36 aa, synthetic construct
SLQSGVPSRFSGSGSGTDFTLTISSLQPEDVGVYYC

SEQ ID NO: 15 - protein, 10 aa, synthetic construct
FGQGTKVDIK

SEQ ID NO: 16 - protein, 212 aa, synthetic construct
DIQMTQSPSSLSASVGDRVTITCRASQSISSYLNWYQQKPGKAPKLLIYAASSLQSGVPSRFSGSGSGTDFTLTISSLQPEDVGVYYCMQALQTLFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 8 aa, synthetic construct
GFTFSDYY

SEQ ID NO: 18 - protein, 8 aa, synthetic construct
ISSSGSTI

SEQ ID NO: 19 - protein, 14 aa, synthetic construct
ARRGAMGHHDGFDM

SEQ ID NO: 20 - protein, 25 aa, synthetic construct
QVQLVESGGGLVKPGGSLRLSCAAS

SEQ ID NO: 21 - protein, 17 aa, synthetic construct
MSWIRQAPGKGLEWVSY

SEQ ID NO: 22 - protein, 38 aa, synthetic construct; identical to SEQ ID NO: 6

SEQ ID NO: 23 - protein, 11 aa, synthetic construct
WGLGTMVTVSS

SEQ ID NO: 24 - protein, 224 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRGAMGHHDGFDMWGLGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 6 aa, synthetic construct
QSISSY

SEQ ID NO: 26 - skipped

SEQ ID NO: 27 - protein, 10 aa, synthetic construct
QQRSNWPLYT

SEQ ID NO: 28 - protein, 26 aa, synthetic construct [sequence omitted]

SEQ ID NO: 29 - protein, 17 aa, synthetic construct
LNWYQQKPGKAPKLLIY

SEQ ID NO: 30 - protein, 36 aa, synthetic construct
SLQSGVPSRFSGSGSGTDFTLTISSLQPEDVAVYYC

SEQ ID NO: 31 - protein, 10 aa, synthetic construct
FGQGTKLEIK

SEQ ID NO: 32 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCQQRSNWPLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 8 aa, synthetic construct
GFTFSDYY

SEQ ID NO: 34 - protein, 8 aa, synthetic construct
ISSSGSTI

SEQ ID NO: 35 - protein, 15 aa, synthetic construct
ARRGGSYYARGAFDI

SEQ ID NO: 36 - protein, 25 aa, synthetic construct
QVQLVESGGGLVKPGGSLRLSCAAS

SEQ ID NO: 37 - protein, 17 aa, synthetic construct
MSWIRQAPGKGLEWVSY

SEQ ID NO: 38 - protein, 38 aa, synthetic construct; identical to SEQ ID NO: 6

SEQ ID NO: 39 - protein, 11 aa, synthetic construct
WGQGTMVTVSS

SEQ ID NO: 40 - protein, 225 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRGGSYYARGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 6 aa, synthetic construct
QSISSY

SEQ ID NO: 42 - skipped

SEQ ID NO: 43 - protein, 11 aa, synthetic construct
QHRSDWPPKFT

SEQ ID NO: 44 - protein, 26 aa, synthetic construct [sequence omitted]

SEQ ID NO: 45 - protein, 17 aa, synthetic construct
LNWYQQKPGKAPKLLIY

SEQ ID NO: 46 - protein, 36 aa, synthetic construct
SLQSGVPSRFSGSGSGTDFTLTISSLQPEDAATYYC

SEQ ID NO: 47 - protein, 10 aa, synthetic construct
FGPGTKVDIK

SEQ ID NO: 48 - protein, 216 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQHRSDWPPKFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 5 aa, synthetic construct
DYDMS

SEQ ID NO: 50 - protein, 16 aa, synthetic construct
EINHSGSTNYNPSLKS

SEQ ID NO: 51 - skipped

SEQ ID NO: 52 - protein, 30 aa, synthetic construct
EVQLVESGGGLVQPGGSLRLSCAASWFDFQ

SEQ ID NO: 53 - protein, 14 aa, synthetic construct
WVRQAPGKGLEWIG

SEQ ID NO: 54 - protein, 32 aa, synthetic construct
RVTISRDNSKNTLYLQMNSLRAEDTAVYYCAK

SEQ ID NO: 55 - protein, 11 aa, synthetic construct
WGQGTLVTVSS

SEQ ID NO: 56 - protein, 111 aa, synthetic construct
EVQLVESGGGLVQPGGSLRLSCAASWFDFQDYDMSWVRQAPGKGLEWIGEINHSGSTNYNPSLKSRVTISRDNSKNTLYLQMNSLRAEDTAVYYCAKIGYWGQGTLVTVSS

SEQ ID NO: 57 - protein, 5 aa, synthetic construct
IYEMS

SEQ ID NO: 58 - protein, 16 aa, synthetic construct
YINHSGSTNYNPSLKS

SEQ ID NO: 59 - protein, 5 aa, synthetic construct
LAPDY

SEQ ID NO: 60 - protein, 30 aa, synthetic construct
EVQLVESGGGLVQPGGSLRLSCAASPFPFT

SEQ ID NO: 61 - protein, 14 aa, synthetic construct
WVRQASGKALEWIG

SEQ ID NO: 62 - protein, 32 aa, synthetic construct
RVTISRDNSKNTLYLQMNSLRAEDTAMYYCAI

SEQ ID NO: 63 - protein, 11 aa, synthetic construct
WGQGTLVTVSS

SEQ ID NO: 64 - protein, 113 aa, synthetic construct
EVQLVESGGGLVQPGGSLRLSCAASPFPFTIYEMSWVRQASGKALEWIGYINHSGSTNYNPSLKSRVTISRDNSKNTLYLQMNSLRAEDTAMYYCAILAPDYWGQGTLVTVSS

SEQ ID NO: 65 - protein, 5 aa, synthetic construct
HYEMS

SEQ ID NO: 66 - protein, 17 aa, synthetic construct
WINTNTGKSTYAQGFTG

SEQ ID NO: 67 - protein, 9 aa, synthetic construct
SGYGYYFDY

SEQ ID NO: 68 - protein, 30 aa, synthetic construct
EVQLVESGGGLVQPGGSLRLSCAASGSPFP

SEQ ID NO: 69 - protein, 14 aa, synthetic construct
WVRQAPGKGLEWMG

SEQ ID NO: 70 - protein, 32 aa, synthetic construct
RFTISRDNSKNTLYLQMNSLRAEDTAVYYCAR

SEQ ID NO: 71 - protein, 11 aa, synthetic construct
WGQGTLVTVSS

SEQ ID NO: 72 - protein, 118 aa, synthetic construct
EVQLVESGGGLVQPGGSLRLSCAASGSPFPHYEMSWVRQAPGKGLEWMGWINTNTGKSTYAQGFTGRFTISRDNSKNTLYLQMNSLRAEDTAVYYCARSGYGYYFDYWGQGTLVTVSS

SEQ ID NO: 73 - protein, 5 aa, synthetic construct
SYAMS

SEQ ID NO: 74 - protein, 16 aa, synthetic construct
EINHSGSTNYNPSLKS

SEQ ID NO: 75 - skipped

SEQ ID NO: 76 - protein, 30 aa, synthetic construct
EVQLVESGGGLVQPGGSLRLSCAASGFTFS

SEQ ID NO: 77 - protein, 14 aa, synthetic construct
WVRQAPGQGLEWIG

SEQ ID NO: 78 - protein, 32 aa, synthetic construct
RVTISRDNSKNTLYLQMNSLRAEDTAVYYCVH

SEQ ID NO: 79 - protein, 11 aa, synthetic construct
WGQGTLVAVSS

SEQ ID NO: 80 - protein, 111 aa, synthetic construct
EVQLVESGGGLVQPGGSLRLSCAASGFTFSSYAMSWVRQAPGQGLEWIGEINHSGSTNYNPSLKSRVTISRDNSKNTLYLQMNSLRAEDTAVYYCVHIDYWGQGTLVAVSS

SEQ ID NO: 81 - protein, 466 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 82 - skipped

SEQ ID NO: 83 - DNA, 660 bp, synthetic construct
  source 1..660: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcaagaggaatcaacttcggtctggacatctggggccaagggaccacggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 84 - DNA, 636 bp, synthetic construct
  source 1..636: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggatgttggggtttattactgcatgcaagctctacaaaccctttttggccaggggaccaaagtggatatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 85 - DNA, 672 bp, synthetic construct
  source 1..672: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagacggggagctatgggccatcatgatggttttgatatgtggggcctagggacaatggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 86 - DNA, 645 bp, synthetic construct
  source 1..645: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcagcagcgtagcaactggcctctgtacacttttggccaggggaccaaactggaaatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 87 - DNA, 675 bp, synthetic construct
  source 1..675: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagacggggtgggagctactatgctcgcggtgcttttgatatctggggccaagggacaatggtcaccgtctcttcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 88 - DNA, 648 bp, synthetic construct
  source 1..648: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcagcaccgtagcgactggcctccgaaattcactttcggccctgggaccaaagtggatatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 89 - DNA, 333 bp, synthetic construct
  source 1..333: mol_type = other DNA
gaggtgcagctggtggagtctgggggaggcttggtacagcctggagggtccctgagactctcctgtgcagcctcttggttcgatttccaggattatgatatgagctgggtccgccaggctccagggaagggtctggagtggattggggaaatcaatcatagtggaagcaccaactacaacccgtccctcaagagtcgagtcaccatctccagagacaactccaagaacacgctgtatctgcaaatgaacagcctgagagccgaggacacggccgtgtattactgtgcgaaaataggctactggggccagggaactctggtcaccgtctcctca

SEQ ID NO: 90 - DNA, 339 bp, synthetic construct
  source 1..339: mol_type = other DNA
gaggtgcagctggtggagtctgggggaggcttggtacagcctggagggtccctgagactctcctgtgcagcctctcccttccctttcaccatttatgaaatgagctgggtccgccaggcttcagggaaggccctggagtggattgggtacatcaatcatagtggaagcaccaactacaacccgtccctcaagagtcgagtcaccatctccagagacaattccaagaacacgctgtatctgcaaatgaacagcctgagagccgaggacacagccatgtattactgtgcgatcctagccccggactactggggccagggaaccctggtcaccgtctcctca

SEQ ID NO: 91 - DNA, 354 bp, synthetic construct
  source 1..354: mol_type = other DNA
gaggtgcagctggtggagtctgggggaggcttggtacagcctggagggtccctgagactctcctgtgcagcctctggctcccccttcccgcattatgaaatgagctgggtccgccaggctccagggaaggggctggagtggatgggatggatcaacaccaacactggaaagtcaacgtatgcccagggcttcacaggacggtttaccatctccagagacaattccaagaacacgctgtatctgcaaatgaacagcctgagagccgaggacacggctgtatattactgtgcacggagcggctatggctactactttgactattggggccagggcaccctggtcaccgtctcctca

SEQ ID NO: 92 - DNA, 333 bp, synthetic construct
  source 1..333: mol_type = other DNA
gaggtgcagctggtggagtctgggggaggcttggtacagcctggagggtccctgagactctcctgtgcagcctctggattcacctttagcagctatgctatgagctgggtccgccaggctccaggacaagggcttgagtggattggggaaatcaatcatagtggaagcaccaactacaacccgtccctcaagagtcgagtcaccatctccagagacaattccaagaacacgctgtatctgcaaatgaacagcctgagagccgaggacacagctgtgtattactgtgtgcacattgactactggggccagggaaccctggtcgccgtctcctca

SEQ ID NO: 93 - protein, 15 aa, synthetic construct
GGGGSGGSGSGGGGS

SEQ ID NO: 94 - protein, 15 aa, synthetic construct
GGGGSGGSGSSGGGS

SEQ ID NO: 95 - protein, 15 aa, synthetic construct
GGGGSGGGGSGGGGS

SEQ ID NO: 96 - protein, 15 aa, synthetic construct
GGGGSGSGASSGGGS

SEQ ID NO: 97 - protein, 15 aa, synthetic construct
GGGGSGSGASGGGGS

SEQ ID NO: 98 - protein, 15 aa, synthetic construct
SGGGSGGGGSGGGGS

SEQ ID NO: 99 - protein, 18 aa, synthetic construct
GSTSGSGKPGSGEGSTKG

SEQ ID NO: 100 - protein, 19 aa, synthetic construct
PNGASQSSSASHTGSAPGS

SEQ ID NO: 101 - protein, 19 aa, synthetic construct
PNGASNSGSAPDTSSAPGS

SEQ ID NO: 102 - protein, 19 aa, synthetic construct
PNGASHSGSAPNTSSAPGS

SEQ ID NO: 103 - protein, 19 aa, synthetic construct
PNGASESGSASKTSSASGS

SEQ ID NO: 104 - protein, 19 aa, synthetic construct
PNGASNSGSAPKTGSASGS

SEQ ID NO: 105 - protein, 19 aa, synthetic construct
PNGASKSGSASQTSSAPGS

SEQ ID NO: 106 - protein, 19 aa, synthetic construct
PNGASHSSSASQTGSAPGS

SEQ ID NO: 107 - protein, 12 aa, synthetic construct
PNGASKRSAPGS

SEQ ID NO: 108 - protein, 19 aa, synthetic construct
PNGASHSGSAPHTSSASGS

SEQ ID NO: 109 - protein, 15 aa, synthetic construct
RGRGRGRGRSRGGGS

SEQ ID NO: 110 - protein, 15 aa, synthetic construct
SHGGSHGGGSGGGGS

SEQ ID NO: 111 - protein, 94 aa, synthetic construct
EVQLVQSGGGLVQPGGSLRLSCSASXMHWVRQAPGKGLEYVSAXYYADSVKGRFTISRDNSKNTLYLQMNSLRAEDTAVYYCXWGQGTMVTVSS

SEQ ID NO: 112 - protein, 25 aa, synthetic construct
EVQLVQSGGGLVQPGGSLRLSCSAS

SEQ ID NO: 113 - protein, 17 aa, synthetic construct
MHWVRQAPGKGLEYVSA

SEQ ID NO: 114 - protein, 38 aa, synthetic construct
YYADSVKGRFTISRDNSKNTLYLQMNSLRAEDTAVYYC

SEQ ID NO: 115 - protein, 11 aa, synthetic construct
WGQGTMVTVSS

SEQ ID NO: 116 - protein, 93 aa, synthetic construct
EATLTQSPSSLSASVGDRVTITCRASXLNWYQQKPGRAPKLLIYXSLQSGVPSRFSGSGSGTDFTLTISSLQPEDFATYYCXFGQGTRLEIKR

SEQ ID NO: 117 - protein, 26 aa, synthetic construct
EATLTQSPSSLSASVGDRVTITCRAS

SEQ ID NO: 118 - protein, 17 aa, synthetic construct
LNWYQQKPGRAPKLLIY

SEQ ID NO: 119 - protein, 36 aa, synthetic construct
SLQSGVPSRFSGSGSGTDFTLTISSLQPEDFATYYC

SEQ ID NO: 120 - protein, 11 aa, synthetic construct
FGQGTRLEIKR

SEQ ID NO: 121 - protein, 94 aa, synthetic construct
EVQLVQSGGGLVQPGGSLRLSCAASXMTWVRQAPGKGLEWVSTXYYADSVKGRFTISRDNDKNTLYLQMNSLRADDTAVYYCXWGQGTLVTVSS

SEQ ID NO: 122 - protein, 25 aa, synthetic construct
EVQLVQSGGGLVQPGGSLRLSCAAS

SEQ ID NO: 123 - protein, 17 aa, synthetic construct
MTWVRQAPGKGLEWVST

SEQ ID NO: 124 - protein, 38 aa, synthetic construct
YYADSVKGRFTISRDNDKNTLYLQMNSLRADDTAVYYC

SEQ ID NO: 125 - protein, 11 aa, synthetic construct
WGQGTLVTVSS

SEQ ID NO: 126 - protein, 92 aa, synthetic construct
QSVVTQPPSASGSPGQSVTISCTGTXVSWYQQHPGKAPKLLIYXERPSGVPDRFSGSKSDNKASLTIIGLQTEDEADYYCXFGTGTKLTVLR

SEQ ID NO: 127 - protein, 25 aa, synthetic construct
QSVVTQPPSASGSPGQSVTISCTGT

SEQ ID NO: 128 - protein, 17 aa, synthetic construct
VSWYQQHPGKAPKLLIY

SEQ ID NO: 129 - protein, 36 aa, synthetic construct
ERPSGVPDRFSGSKSDNKASLTIIGLQTEDEADYYC

SEQ ID NO: 130 - protein, 11 aa, synthetic construct
FGTGTKLTVLR

SEQ ID NO: 131 - protein, 8 aa, synthetic construct
GFTFDDYA

SEQ ID NO: 132 - protein, 8 aa, synthetic construct
GFTFSSYD

SEQ ID NO: 133 - protein, 8 aa, synthetic construct
GYTFTGYY

SEQ ID NO: 134 - protein, 8 aa, synthetic construct
GFTFSSYW

SEQ ID NO: 135 - protein, 8 aa, synthetic construct
GFTFSGSA

SEQ ID NO: 136 - protein, 8 aa, synthetic construct
GFTFSSYA

SEQ ID NO: 137 - protein, 8 aa, synthetic construct
GFTFGDYA

SEQ ID NO: 138 - protein, 8 aa, synthetic construct
GFTVSSNE

SEQ ID NO: 139 - protein, 8 aa, synthetic construct
GFTFSSYG

SEQ ID NO: 140 - protein, 8 aa, synthetic construct
GFTFSYYY

SEQ ID NO: 141 - protein, 7 aa, synthetic construct
IGTAGDT

SEQ ID NO: 142 - protein, 8 aa, synthetic construct
IKQDGSEK

SEQ ID NO: 143 - protein, 10 aa, synthetic construct
IKSKTDGGTT

SEQ ID NO: 144 - protein, 8 aa, synthetic construct
ISGSGGST

SEQ ID NO: 145 - protein, 8 aa, synthetic construct
ISYDGSNK

SEQ ID NO: 146 - protein, 6 aa, synthetic construct
ISGGST

SEQ ID NO: 147 - protein, 8 aa, synthetic construct
ISSSSSTI

SEQ ID NO: 148 - protein, 8 aa, synthetic construct
ISWNSGSI

SEQ ID NO: 149 - protein, 8 aa, synthetic construct
IKQDGSEK

SEQ ID NO: 150 - protein, 8 aa, synthetic construct
ISAYNGNT

SEQ ID NO: 151 - protein, 10 aa, synthetic construct
ATGINFGLSI

SEQ ID NO: 152 - protein, 10 aa, synthetic construct
AAGINFGLDI

SEQ ID NO: 153 - protein, 10 aa, synthetic construct
VRGINFGLYI

SEQ ID NO: 154 - protein, 11 aa, synthetic construct
AHRGINFGLDI

SEQ ID NO: 155 - protein, 11 aa, synthetic construct
ARRGINFGLYI

SEQ ID NO: 156 - protein, 11 aa, synthetic construct
ARIGINFGLDI

SEQ ID NO: 157 - protein, 11 aa, synthetic construct
AKDGINFGLDI

SEQ ID NO: 158 - protein, 10 aa, synthetic construct
TTGINFGLYI

SEQ ID NO: 159 - protein, 10 aa, synthetic construct
SRGINFGLDI

SEQ ID NO: 160 - protein, 10 aa, synthetic construct
AKGINFGLSI

SEQ ID NO: 161 - protein, 6 aa, synthetic construct
QSISSW

SEQ ID NO: 162 - protein, 6 aa, synthetic construct
QGISSY

SEQ ID NO: 163 - protein, 6 aa, synthetic construct
QGISNY

SEQ ID NO: 164 - protein, 6 aa, synthetic construct
QGISSW

SEQ ID NO: 165 - protein, 6 aa, synthetic construct
QGISSA

SEQ ID NO: 166 - protein, 6 aa, synthetic construct
QSVSSY

SEQ ID NO: 167 - protein, 6 aa, synthetic construct
QSVSSN

SEQ ID NO: 168 - protein, 7 aa, synthetic construct
QSVSSSY

SEQ ID NO: 169 - protein, 6 aa, synthetic construct
QSIGSS

SEQ ID NO: 170 - protein, 7 aa, synthetic construct
QSVSSSY

SEQ ID NO: 171-180 - skipped (10 entries)

SEQ ID NO: 181 - protein, 7 aa, synthetic construct
MQALQTL

SEQ ID NO: 182 - protein, 7 aa, synthetic construct
QQALQTL

SEQ ID NO: 183 - protein, 7 aa, synthetic construct
LQALQTL

SEQ ID NO: 184 - protein, 7 aa, synthetic construct
QKALQTL

SEQ ID NO: 185 - protein, 7 aa, synthetic construct
MQALQSL

SEQ ID NO: 186 - protein, 7 aa, synthetic construct
MQALPTL

SEQ ID NO: 187 - protein, 7 aa, synthetic construct
HQALQTL

SEQ ID NO: 188 - protein, 7 aa, synthetic construct
TQALQTL

SEQ ID NO: 189 - protein, 7 aa, synthetic construct
EQALQTL

SEQ ID NO: 190 - protein, 7 aa, synthetic construct
KQALQTL

SEQ ID NO: 191 - protein, 8 aa, synthetic construct
GFTFDDYA

SEQ ID NO: 192 - protein, 8 aa, synthetic construct
GFTFSSYD

SEQ ID NO: 193 - protein, 8 aa, synthetic construct
GYTFTGYY

SEQ ID NO: 194 - protein, 8 aa, synthetic construct
GFTFSSYW

SEQ ID NO: 195 - protein, 8 aa, synthetic construct
GFTFSGSA

SEQ ID NO: 196 - protein, 8 aa, synthetic construct
GFTFSSYA

SEQ ID NO: 197 - protein, 8 aa, synthetic construct
GFTFGDYA

SEQ ID NO: 198 - protein, 8 aa, synthetic construct
GFTVSSNE

SEQ ID NO: 199 - protein, 8 aa, synthetic construct
GFTFSSYG

SEQ ID NO: 200 - protein, 8 aa, synthetic construct
GFTFSYYY

SEQ ID NO: 201 - protein, 7 aa, synthetic construct
IGTAGDT

SEQ ID NO: 202 - protein, 8 aa, synthetic construct
IKQDGSEK

SEQ ID NO: 203 - protein, 10 aa, synthetic construct
IKSKTDGGTT

SEQ ID NO: 204 - protein, 8 aa, synthetic construct
ISGSGGST

SEQ ID NO: 205 - protein, 8 aa, synthetic construct
ISYDGSNK

SEQ ID NO: 206 - protein, 6 aa, synthetic construct
ISGGST

SEQ ID NO: 207 - protein, 8 aa, synthetic construct
ISSSSSTI

SEQ ID NO: 208 - protein, 8 aa, synthetic construct
ISWNSGSI

SEQ ID NO: 209 - protein, 8 aa, synthetic construct
IKQDGSEK

SEQ ID NO: 210 - protein, 8 aa, synthetic construct
ISAYNGNT

SEQ ID NO: 211 - protein, 14 aa, synthetic construct
YRRGAMGHHDGFDM

SEQ ID NO: 212 - protein, 14 aa, synthetic construct
ARRGAMGHHDGFSM

SEQ ID NO: 213 - protein, 14 aa, synthetic construct
ARRGYMGHHDGFDM

SEQ ID NO: 214 - protein, 14 aa, synthetic construct
ARRGAMGHHSGFDM

SEQ ID NO: 215 - protein, 14 aa, synthetic construct
SRRGAMGHHDGFDM

SEQ ID NO: 216 - protein, 14 aa, synthetic construct
VRRGAMGHHDGFDM

SEQ ID NO: 217 - protein, 14 aa, synthetic construct
AKRGAMGHHDGFDM

SEQ ID NO: 218 - protein, 14 aa, synthetic construct
AGRGAMGHHDGFDM

SEQ ID NO: 219 - protein, 15 aa, synthetic construct
ARIRGAMGHHDGFDM

SEQ ID NO: 220 - protein, 15 aa, synthetic construct
AHRRGAMGHHDGFDM

SEQ ID NO: 221 - protein, 6 aa, synthetic construct
QSISSW

SEQ ID NO: 222 - protein, 6 aa, synthetic construct
QGISSY

SEQ ID NO: 223 - protein, 6 aa, synthetic construct
QGISNY

SEQ ID NO: 224 - protein, 6 aa, synthetic construct
QGISSW

SEQ ID NO: 225 - protein, 6 aa, synthetic construct
QGISSA

SEQ ID NO: 226 - protein, 6 aa, synthetic construct
QSVSSY

SEQ ID NO: 227 - protein, 6 aa, synthetic construct
QSVSSN

SEQ ID NO: 228 - protein, 7 aa, synthetic construct
QSVSSSY

SEQ ID NO: 229 - protein, 6 aa, synthetic construct
QSIGSS

SEQ ID NO: 230 - protein, 7 aa, synthetic construct
QSVSSSY

SEQ ID NO: 231-240 - skipped (10 entries)

SEQ ID NO: 241 - protein, 10 aa, synthetic construct
MQRSNWPLYT

SEQ ID NO: 242 - protein, 10 aa, synthetic construct
HQRSNWPLYT

SEQ ID NO: 243 - protein, 10 aa, synthetic construct
LQRSNWPLYT

SEQ ID NO: 244 - protein, 10 aa, synthetic construct
QQRTNWPLYT

SEQ ID NO: 245 - protein, 10 aa, synthetic construct
QQRSNWPLYS

SEQ ID NO: 246 - protein, 10 aa, synthetic construct
TQRSNWPLYT

SEQ ID NO: 247 - protein, 10 aa, synthetic construct
EQRSNWPLYT

SEQ ID NO: 248 - protein, 10 aa, synthetic construct
KQRSNWPLYT

SEQ ID NO: 249 - protein, 9 aa, synthetic construct
QRSNWPLYT

SEQ ID NO: 250 - protein, 10 aa, synthetic construct
QQRANWPLYT

SEQ ID NO: 251 - protein, 8 aa, synthetic construct
GFTFDDYA

SEQ ID NO: 252 - protein, 8 aa, synthetic construct
GFTFSSYD

SEQ ID NO: 253 - protein, 8 aa, synthetic construct
GYTFTGYY

SEQ ID NO: 254 - protein, 8 aa, synthetic construct
GFTFSSYW

SEQ ID NO: 255 - protein, 8 aa, synthetic construct
GFTFSGSA

SEQ ID NO: 256 - protein, 8 aa, synthetic construct
GFTFSSYA

SEQ ID NO: 257 - protein, 8 aa, synthetic construct
GFTFGDYA

SEQ ID NO: 258 - protein, 8 aa, synthetic construct
GFTVSSNE

SEQ ID NO: 259 - protein, 8 aa, synthetic construct
GFTFSSYG

SEQ ID NO: 260 - protein, 8 aa, synthetic construct
GFTFSYYY

SEQ ID NO: 261 - protein, 7 aa, synthetic construct
IGTAGDT

SEQ ID NO: 262 - protein, 8 aa, synthetic construct
IKQDGSEK

SEQ ID NO: 263 - protein, 10 aa, synthetic construct
IKSKTDGGTT

SEQ ID NO: 264 - protein, 8 aa, synthetic construct
ISGSGGST

SEQ ID NO: 265 - protein, 8 aa, synthetic construct
ISYDGSNK

SEQ ID NO: 266 - protein, 6 aa, synthetic construct
ISGGST

SEQ ID NO: 267 - protein, 8 aa, synthetic construct
ISSSSSTI

SEQ ID NO: 268 - protein, 8 aa, synthetic construct
ISWNSGSI

SEQ ID NO: 269 - protein, 8 aa, synthetic construct
IKQDGSEK

SEQ ID NO: 270 - protein, 8 aa, synthetic construct
ISAYNGNT

SEQ ID NO: 271 - protein, 15 aa, synthetic construct
VRRGGSYYARGAFDI

SEQ ID NO: 272 - protein, 15 aa, synthetic construct
SRRGGSYYARGAFDI

SEQ ID NO: 273 - protein, 15 aa, synthetic construct
MRRGGSYYARGAFDI

SEQ ID NO: 274 - protein, 15 aa, synthetic construct
TRRGGSYYARGAFDI

SEQ ID NO: 275 - protein, 14 aa, synthetic construct
ARGGSYYARGAFDI

SEQ ID NO: 276 - protein, 14 aa, synthetic construct
ARRGSYYARGAFDI

SEQ ID NO: 277 - protein, 14 aa, synthetic construct
ARRGGSYARGAFDI

SEQ ID NO: 278 - protein, 15 aa, synthetic construct
ARRGGSYYDRGAFDI

SEQ ID NO: 279 - protein, 15 aa, synthetic construct
ARRGGSYYARGAFSI

SEQ ID NO: 280 - protein, 15 aa, synthetic construct
ARRGGSYYARGYFDI

SEQ ID NO: 281 - protein, 6 aa, synthetic construct
QSISSW

SEQ ID NO: 282 - protein, 6 aa, synthetic construct
QGISSY

SEQ ID NO: 283 - protein, 6 aa, synthetic construct
QGISNY

SEQ ID NO: 284 - protein, 6 aa, synthetic construct
QGISSW

SEQ ID NO: 285 - protein, 6 aa, synthetic construct
QGISSA

SEQ ID NO: 286 - protein, 6 aa, synthetic construct
QSVSSY

SEQ ID NO: 287 - protein, 6 aa, synthetic construct
QSVSSN

SEQ ID NO: 288 - protein, 7 aa, synthetic construct
QSVSSSY

SEQ ID NO: 289 - protein, 6 aa, synthetic construct
QSIGSS

SEQ ID NO: 290 - protein, 7 aa, synthetic construct
QSVSSSY

SEQ ID NO: 291-300 - skipped (10 entries)

SEQ ID NO: 301 - protein, 11 aa, synthetic construct
QQRSDWPPKFT

SEQ ID NO: 302 - protein, 11 aa, synthetic construct
LQRSDWPPKFT

SEQ ID NO: 303 - protein, 11 aa, synthetic construct
QKRSDWPPKFT

SEQ ID NO: 304 - protein, 12 aa, synthetic construct
EQHRSDWPPKFT

SEQ ID NO: 305 - protein, 12 aa, synthetic construct
MQHRSDWPPKFT

SEQ ID NO: 306 - protein, 12 aa, synthetic construct
TQHRSDWPPKFT

SEQ ID NO: 307 - protein, 12 aa, synthetic construct
QQHRSDWPPKFT

SEQ ID NO: 308 - protein, 11 aa, synthetic construct
QHRTDWPPKFT

SEQ ID NO: 309 - protein, 11 aa, synthetic construct
QHRSDWPPKFS

SEQ ID NO: 310 - protein, 11 aa, synthetic construct
QHRSDWPPKDT

SEQ ID NO: 311 - protein, 5 aa, synthetic construct
DYDMH

SEQ ID NO: 312 - protein, 5 aa, synthetic construct
DYDMG

SEQ ID NO: 313 - protein, 5 aa, synthetic construct
DYAMS

SEQ ID NO: 314 - protein, 5 aa, synthetic construct
DYYMS

SEQ ID NO: 315 - protein, 5 aa, synthetic construct
SYDMS

SEQ ID NO: 316 - protein, 5 aa, synthetic construct
DYYMH

SEQ ID NO: 317 - protein, 5 aa, synthetic construct
DYYMN

SEQ ID NO: 318 - protein, 5 aa, synthetic construct
SYDMN

SEQ ID NO: 319 - protein, 5 aa, synthetic construct
SYDMH

SEQ ID NO: 320 - protein, 5 aa, synthetic construct
DYDMD

SEQ ID NO: 321 - protein, 16 aa, synthetic construct
DINHSGSTNYNPSLKS

SEQ ID NO: 322 - protein, 16 aa, synthetic construct
EINHSGSTNYNPSLRS

SEQ ID NO: 323 - protein, 16 aa, synthetic construct
EINHSGSTNYNPSLKG

SEQ ID NO: 324 - protein, 16 aa, synthetic construct
EINHSGSTYYNPSLKS

SEQ ID NO: 325 - protein, 16 aa, synthetic construct
EINHSGSTYYNPSLKG

SEQ ID NO: 326 - protein, 16 aa, synthetic construct
EINHSGSTNYNPSVKS

SEQ ID NO: 327 - protein, 16 aa, synthetic construct
EINHSGSTNYNPSVKG

SEQ ID NO: 328 - protein, 16 aa, synthetic construct
EIYHSGSTNYNPSLKS

SEQ ID NO: 329 - protein, 16 aa, synthetic construct
EIYHSGSTNYNPSLKG

SEQ ID NO: 330 - protein, 16 aa, synthetic construct
DIYHSGSTNYNPSLKG

SEQ ID NO: 331-340 - skipped (10 entries)

SEQ ID NO: 341 - protein, 5 aa, synthetic construct
IYEMH

SEQ ID NO: 342 - protein, 5 aa, synthetic construct
IYEMD

SEQ ID NO: 343 - protein, 5 aa, synthetic construct
IYEMG

SEQ ID NO: 344 - protein, 5 aa, synthetic construct
IYEMN

SEQ ID NO: 345 - protein, 5 aa, synthetic construct
IYGMS

SEQ ID NO: 346 - protein, 5 aa, synthetic construct
IYGMH

SEQ ID NO: 347 - protein, 5 aa, synthetic construct
IYGMD

SEQ ID NO: 348 - protein, 5 aa, synthetic construct
IYGMG

SEQ ID NO: 349 - protein, 5 aa, synthetic construct
IYGMN

SEQ ID NO: 350 - protein, 5 aa, synthetic construct
LYEMS

SEQ ID NO: 351 - protein, 16 aa, synthetic construct
YIYHSGSTNYNPSLKS

SEQ ID NO: 352 - protein, 16 aa, synthetic construct
YINHSGSTNYNPSLKG

SEQ ID NO: 353 - protein, 16 aa, synthetic construct
YINHSGSTNYNPSVKS

SEQ ID NO: 354 - protein, 16 aa, synthetic construct
YIYHSGSTNYNPSLKG

SEQ ID NO: 355 - protein, 16 aa, synthetic construct
YIYHSGSTNYNPSVKS

SEQ ID NO: 356 - protein, 16 aa, synthetic construct
YIYHSGSTYYNPSLKS

SEQ ID NO: 357 - protein, 16 aa, synthetic construct
YINHSGSTYYNPSLKS

SEQ ID NO: 358 - protein, 16 aa, synthetic construct
YINHSGSTYYNPSLKG

SEQ ID NO: 359 - protein, 16 aa, synthetic construct
YINHSGSTYYNPSVKS

SEQ ID NO: 360 - protein, 16 aa, synthetic construct
YINHGGSTNYNPSLKS

SEQ ID NO: 361 - protein, 5 aa, synthetic construct
LAPEY

SEQ ID NO: 362 - protein, 5 aa, synthetic construct
IAPEY

SEQ ID NO: 363 - protein, 5 aa, synthetic construct
IAPDY

SEQ ID NO: 364 - protein, 5 aa, synthetic construct
LVPDY

SEQ ID NO: 365 - protein, 5 aa, synthetic construct
IVPDY

SEQ ID NO: 366 - protein, 5 aa, synthetic construct
LVPEY

SEQ ID NO: 367 - protein, 5 aa, synthetic construct
VAPDY

SEQ ID NO: 368 - protein, 5 aa, synthetic construct
VAPEY

SEQ ID NO: 369 - protein, 5 aa, synthetic construct
VVPDY

SEQ ID NO: 370 - protein, 5 aa, synthetic construct
LIPDY

SEQ ID NO: 371 - protein, 5 aa, synthetic construct
HYDMS

SEQ ID NO: 372 - protein, 5 aa, synthetic construct
HYEMH

SEQ ID NO: 373 - protein, 5 aa, synthetic construct
HYEMG

SEQ ID NO: 374 - protein, 5 aa, synthetic construct
HYEMD

SEQ ID NO: 375 - protein, 5 aa, synthetic construct
HYEMN

SEQ ID NO: 376 - protein, 5 aa, synthetic construct
HYDMG

SEQ ID NO: 377 - protein, 5 aa, synthetic construct
HYDMH

SEQ ID NO: 378 - protein, 5 aa, synthetic construct
HYDMD

SEQ ID NO: 379 - protein, 5 aa, synthetic construct
HYDMN

SEQ ID NO: 380 - protein, 5 aa, synthetic construct
DYEMG

SEQ ID NO: 381 - protein, 17 aa, synthetic construct
WINPNTGKSTYAQGFTG

SEQ ID NO: 382 - protein, 17 aa, synthetic construct
WINTNTGKSTYAQGFQG

SEQ ID NO: 383 - protein, 17 aa, synthetic construct
WINTNTGKSTYAQKFTG

SEQ ID NO: 384 - protein, 17 aa, synthetic construct
WINTNTGKSTYSQGFTG

SEQ ID NO: 385 - protein, 17 aa, synthetic construct
WINTNTGKSNYAQGFTG

SEQ ID NO: 386 - protein, 17 aa, synthetic construct
WINTNSGKSTYAQGFTG

SEQ ID NO: 387 - protein, 17 aa, synthetic construct
WINTNTNKSTYAQGFTG

SEQ ID NO: 388 - protein, 17 aa, synthetic construct
WLNTNTGKSTYAQGFTG

SEQ ID NO: 389 - protein, 17 aa, synthetic construct
WINTNTGKSTYAQGFTS

SEQ ID NO: 390 - protein, 17 aa, synthetic construct
WINTNTGKSTYANGFTG

SEQ ID NO: 391 - protein, 9 aa, synthetic construct
TGYGYYFDY

SEQ ID NO: 392 - protein, 9 aa, synthetic construct
SGYGYYFEY

SEQ ID NO: 393 - protein, 9 aa, synthetic construct
SSYGYYFDY

SEQ ID NO: 394 - protein, 9 aa, synthetic construct
SGYSYYFDY

SEQ ID NO: 395 - protein, 9 aa, synthetic construct
SSYGYYFEY

SEQ ID NO: 396 - protein, 9 aa, synthetic construct
SGYSYYFEY

SEQ ID NO: 397 - protein, 9 aa, synthetic construct
TGYSYYFEY

SEQ ID NO: 398 - protein, 9 aa, synthetic construct
TSYSYYFEY

SEQ ID NO: 399 - protein, 9 aa, synthetic construct
TTYSYYFEY

SEQ ID NO: 400 - protein, 9 aa, synthetic construct
TTYSYYFDY

SEQ ID NO: 401 - protein, 5 aa, synthetic construct
SYAMG

SEQ ID NO: 402 - protein, 5 aa, synthetic construct
SYAMD

SEQ ID NO: 403 - protein, 5 aa, synthetic construct
SYAMN

SEQ ID NO: 404 - protein, 5 aa, synthetic construct
SYAMH

SEQ ID NO: 405 - protein, 5 aa, synthetic construct
SYEMS

SEQ ID NO: 406 - protein, 5 aa, synthetic construct
SYEMG

SEQ ID NO: 407 - protein, 5 aa, synthetic construct
SYEMH

SEQ ID NO: 408 - protein, 5 aa, synthetic construct
SYEMD

SEQ ID NO: 409 - protein, 5 aa, synthetic construct
TYAMS

SEQ ID NO: 410 - protein, 5 aa, synthetic construct
SYEMD

SEQ ID NO: 411 - protein, 16 aa, synthetic construct
EINHNGSTNYNPSLKS

SEQ ID NO: 412 - protein, 16 aa, synthetic construct
EINHSGSTNYNPSLKG

SEQ ID NO: 413 - protein, 16 aa, synthetic construct
EINHSGSTNYNPSVKS

SEQ ID NO: 414 - protein, 16 aa, synthetic construct
EINHSGSTYYNPSLKS

SEQ ID NO: 415 - protein, 16 aa, synthetic construct
DINHSGSTNYNPSLKS

SEQ ID NO: 416 - protein, 16 aa, synthetic construct
EINHSGTTNYNPSLKS

SEQ ID NO: 417 - protein, 16 aa, synthetic construct
DINHSGTTNYNPSLKS

SEQ ID NO: 418 - protein, 16 aa, synthetic construct
EINHSGTTNYNPSLKG

SEQ ID NO: 419 - protein, 16 aa, synthetic construct
EINHSGTSNYNPSLKS

SEQ ID NO: 420 - protein, 16 aa, synthetic construct
EINHSGTSNYNPSLKG

SEQ ID NO: 421-430 - skipped (10 entries)

SEQ ID NO: 431 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 432 - protein, 107 aa, synthetic construct [sequence omitted]

SEQ ID NO: 433 - protein, 15 aa, synthetic construct
EPKSCDKTHTCPPCP

SEQ ID NO: 434 - protein, 15 aa, synthetic construct
DKTHTCPPCPAPPVA

SEQ ID NO: 435 - protein, 12 aa, synthetic construct
ESKYGPPCPPCP

SEQ ID NO: 436 - protein, 49 aa, synthetic construct
KPTTTPAPRPPTPAPTIASQPLSLRPEACRPAAGGAVHTRGLDFACDIY

SEQ ID NO: 437 - protein, 39 aa, synthetic construct
IEVMYPPPYLDNERSNGTIIHVKGKHLCPSPLFPGPSKP

SEQ ID NO: 438 - protein, 5 aa, synthetic construct
GGGGS

SEQ ID NO: 439 - skipped

SEQ ID NO: 440 - protein, 21 aa, synthetic construct
LCYLLDGILFIYGVILTALFL

SEQ ID NO: 441 - protein, 22 aa, synthetic construct
MALIVLGGVAGLLLFIGLGIFF

SEQ ID NO: 442 - protein, 21 aa, synthetic construct
IYIWAPLAGTCGVLLLSLVIT

SEQ ID NO: 443 - protein, 27 aa, synthetic construct
FWVLVVVGGVLACYSLLVTVAFIIFWV

SEQ ID NO: 444 - protein, 21 aa, synthetic construct
FWLPIGCAAFVVVCILGCILI

SEQ ID NO: 445 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 446 - protein, 42 aa, synthetic construct
KRGRKKLLYIFKQPFMRPVQTTQEEDGCSCRFPEEEEGGCEL

SEQ ID NO: 447 - protein, 41 aa, synthetic construct
RSKRSRLLHSDYMNMTPRRPGPTRKHYQPYAPPRDFAAYRS

SEQ ID NO: 448 - protein, 42 aa, synthetic construct
ALYLLRRDQRLPPDAHKPPGGGSFRTPIQEEQADAHSTLAKI

SEQ ID NO: 449 - protein, 38 aa, synthetic construct
CWLTKKKYSSSVHDPNGEYMFMRAVNTAKKSRLTDVTL

SEQ ID NO: 450 - protein, 24 aa, synthetic construct
LCARPRRSPAQEDGKVYINMPGRG

SEQ ID NO: 451 - protein, 52 aa, synthetic construct
YFLGRLVPRGRGAAEAATRKQRITETESPYQELQGQRSDVYSDLNTQRPYYK

SEQ ID NO: 452 - protein, 48 aa, synthetic construct
QRRKYRSNKGESPVEPAEPCHYSCPREEEGSTIPIQEDYRKPEPACSP

SEQ ID NO: 453 - protein, 119 aa, synthetic construct [sequence omitted]

SEQ ID NO: 454 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 455 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 456 - protein, 108 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 457 - protein, 117 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 458 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 459 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 460 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 461 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 462 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 463 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 464 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 465 - protein, 107 aa, synthetic construct [sequence omitted]

SEQ ID NO: 466 - protein, 114 aa, synthetic construct
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 467 - protein, 112 aa, synthetic construct
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 468 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 469 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 470 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 471 - protein, 109 aa, Homo sapiens
APYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 472 - protein, 14 aa, synthetic construct
GGGGSGGGSGGGGS

SEQ ID NO: 473 - protein, 8 aa, synthetic construct
GFTFSDYY

SEQ ID NO: 474 - protein, 8 aa, synthetic construct
ISSSGSTI

SEQ ID NO: 475 - protein, 16 aa, synthetic construct
ARDAPTSGWKWFAPDY

SEQ ID NO: 476 - protein, 25 aa, synthetic construct
QVQLVESGGGLVKPGGSLRLSCAAS

SEQ ID NO: 477 - protein, 17 aa, synthetic construct
MSWIRQAPGKGLEWVSY

SEQ ID NO: 478 - protein, 38 aa, synthetic construct; identical to SEQ ID NO: 6

SEQ ID NO: 479 - protein, 11 aa, synthetic construct
WGQGALVTVSS

SEQ ID NO: 480 - protein, 226 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDAPTSGWKWFAPDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 481 - protein, 6 aa, synthetic construct
QSISSY

SEQ ID NO: 482 - skipped

SEQ ID NO: 483 - protein, 9 aa, synthetic construct
QQSYSTPRT

SEQ ID NO: 484 - protein, 26 aa, synthetic construct [sequence omitted]

SEQ ID NO: 485 - protein, 17 aa, synthetic construct
LNWYQQKPGKAPKLLIY

SEQ ID NO: 486 - protein, 36 aa, synthetic construct; identical to SEQ ID NO: 46

SEQ ID NO: 487 - protein, 10 aa, synthetic construct
FGQGTKLEIK

SEQ ID NO: 488 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDAATYYCQQSYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 489 - DNA, 678 bp, synthetic construct
  source 1..678: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagagatgcccctactagtggctggaagtggtttgcccctgactactggggccagggagccctggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgt

SEQ ID NO: 490 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcaacagagttacagtacccctcgaacttttggccaggggaccaaactggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 491 - protein, 16 aa, synthetic construct
ARDAPTSGWKWFYPDY

SEQ ID NO: 492 - protein, 16 aa, synthetic construct
ARDYPTSGWKWFAPDY

SEQ ID NO: 493 - protein, 16 aa, synthetic construct
ARDAPSSGWKWFAPDY

SEQ ID NO: 494 - protein, 16 aa, synthetic construct
ARDAPTSGWKWFDPDY

SEQ ID NO: 495 - protein, 16 aa, synthetic construct
ARDAPESGWKWFAPDY

SEQ ID NO: 496 - protein, 16 aa, synthetic construct
ARDAPTSGWKWFSPDY

SEQ ID NO: 497 - protein, 16 aa, synthetic construct
ARDAPTDGWKWFAPDY

SEQ ID NO: 498 - protein, 16 aa, synthetic construct
ARDAPTSGWKWFAPDA

SEQ ID NO: 499 - protein, 16 aa, synthetic construct
SRDAPTSGWKWFAPDY

SEQ ID NO: 500 - protein, 16 aa, synthetic construct
ARDAPTSGWKWFAPDS

SEQ ID NO: 501 - protein, 9 aa, synthetic construct
QQSYSEPRT

SEQ ID NO: 502 - protein, 9 aa, synthetic construct
QQSYSTPRE

SEQ ID NO: 503 - protein, 9 aa, synthetic construct
QQAYSTPRT

SEQ ID NO: 504 - protein, 9 aa, synthetic construct
QQSYDTPRT

SEQ ID NO: 505 - protein, 9 aa, synthetic construct
QQDYSTPRT

SEQ ID NO: 506 - protein, 9 aa, synthetic construct
QQSYATPRT

SEQ ID NO: 507 - protein, 9 aa, synthetic construct
QQAYSTPRE

SEQ ID NO: 508 - protein, 9 aa, synthetic construct
QQSYDTPRE

SEQ ID NO: 509 - protein, 9 aa, synthetic construct
QQYYSTPRT

SEQ ID NO: 510 - protein, 9 aa, synthetic construct
QQSASTPRT

SEQ ID NO: 511 - protein, 47 aa, synthetic construct
TTTPAPRPPTPAPTIASQPLSLRPEACRPAAGGAVHTRGLDFACDIY

SEQ ID NO: 512 - protein, 45 aa, synthetic construct
TTTPAPRPPTPAPTIASQPLSLRPEACRPAAGGAVHTRGLDFACD

SEQ ID NO: 513 - protein, 24 aa, synthetic construct
IYIWAPLAGTCGVLLLSLVITLYC

SEQ ID NO: 514 - protein, 22 aa, synthetic construct
IWAPLAGTCGVLLLSLVITLYC

SEQ ID NO: 515 - protein, 19 aa, synthetic construct
IWAPLAGTCGVLLLSLVIT

SEQ ID NO: 516 - protein, 5 aa, synthetic construct
GQAGR

SEQ ID NO: 517 - protein, 8 aa, synthetic construct
GGGSGGGG

SEQ ID NO: 518 - protein, 5 aa, synthetic construct
SGGGG

SEQ ID NO: 519 - protein, 5 aa, synthetic construct
GGGGS

SEQ ID NO: 520 - protein, 30 aa, synthetic construct
GGGGSGGGGSGGGGSGGGGSGGGGSGGGGS

SEQ ID NO: 521 - protein, 458 aa, synthetic construct
DIQMTQSPSSLSASVGDRVTITCRASQSISSYLNWYQQKPGKAPKLLIYAASSLQSGVPSRFSGSGSGTDFTLTISSLQPEDVGVYYCMQALQTLFGQGTKVDIKGGGSGGGGQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGINFGLDIWGQGTTVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 522 - DNA, 1377 bp, synthetic construct
  source 1..1377: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggatgttggggtttattactgcatgcaagctctacaaaccctttttggccaggggaccaaagtggatatcaaaggtggcgggtctggtggaggcggacaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcaagaggaatcaacttcggtctggacatctggggccaagggaccacggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 523 - protein, 462 aa, synthetic construct
DIQMTQSPSSLSASVGDRVTITCRASQSISSYLNWYQQKPGKAPKLLIYAASSLQSGVPSRFSGSGSGTDFTLTISSLQPEDVAVYYCQQRSNWPLYTFGQGTKLEIKSGGGGQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRGAMGHHDGFDMWGLGTMVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 524 - DNA, 1389 bp, synthetic construct
  source 1..1389: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgtggcagtttattactgtcagcagcgtagcaactggcctctgtacacttttggccaggggaccaaactggaaatcaaatctggtggaggcggacaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagacggggagctatgggccatcatgatggttttgatatgtggggcctagggacaatggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 525 - protein, 464 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRGGSYYARGAFDIWGQGTMVTVSSGGGGSDIQMTQSPSSLSASVGDRVTITCRASQSISSYLNWYQQKPGKAPKLLIYAASSLQSGVPSRFSGSGSGTDFTLTISSLQPEDAATYYCQHRSDWPPKFTFGPGTKVDIKGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 526 - DNA, 1395 bp, synthetic construct
  source 1..1395: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccgtgtattactgtgcgagacggggtgggagctactatgctcgcggtgcttttgatatctggggccaagggacaatggtcaccgtctcttcaggaggtggcgggtctgatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaagatgctgcaacgtattactgtcagcaccgtagcgactggcctccgaaattcactttcggccctgggaccaaagtggatatcaaaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 527 - protein, 271 aa, synthetic construct
GGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 528 - DNA, 813 bp, synthetic construct
  source 1..813: mol_type = other DNA
ggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 529 - protein, 5 aa, synthetic construct
SYAMH

SEQ ID NO: 530 - protein, 16 aa, synthetic construct
TIGPRGINTHYADSVK

SEQ ID NO: 531 - protein, 12 aa, synthetic construct
DRVGYNSYGMDV

SEQ ID NO: 532 - protein, 30 aa, synthetic construct
EVQLVESGGGVVQPGRSLRLSCAASGFTFS

SEQ ID NO: 533 - protein, 14 aa, synthetic construct
WVRQAPGQGLEWVS

SEQ ID NO: 534 - protein, 33 aa, synthetic construct
GRFTISRDNSKNTLYLQMNSLRAEDTATYYCAR

SEQ ID NO: 535 - protein, 15 aa, synthetic construct
WGQGTMVTVSSGQAG

SEQ ID NO: 536 - protein, 125 aa, synthetic construct
EVQLVESGGGVVQPGRSLRLSCAASGFTFSSYAMHWVRQAPGQGLEWVSTIGPRGINTHYADSVKGRFTISRDNSKNTLYLQMNSLRAEDTATYYCARDRVGYNSYGMDVWGQGTMVTVSSGQAG

SEQ ID NO: 537 - protein, 5 aa, synthetic construct
SYAMH

SEQ ID NO: 538 - protein, 15 aa, synthetic construct
EIYHGGSTDYNPSLK

SEQ ID NO: 539 - protein, 13 aa, synthetic construct
GGKRLLSVYGVDV

SEQ ID NO: 540 - protein, 30 aa, synthetic construct; identical to SEQ ID NO: 532

SEQ ID NO: 541 - protein, 14 aa, synthetic construct
WVRQAPGKGLEWIG

SEQ ID NO: 542 - protein, 33 aa, synthetic construct
SRVTISRDDSTNTLYLQMNSLRAEDTAVYYCTR

SEQ ID NO: 543 - protein, 11 aa, synthetic construct
WGQGTMVTVSS

SEQ ID NO: 544 - protein, 121 aa, synthetic construct
EVQLVESGGGVVQPGRSLRLSCAASGFTFSSYAMHWVRQAPGKGLEWIGEIYHGGSTDYNPSLKSRVTISRDDSTNTLYLQMNSLRAEDTAVYYCTRGGKRLLSVYGVDVWGQGTMVTVSS

SEQ ID NO: 545 - DNA, 363 bp, synthetic construct
  source 1..363: mol_type = other DNA
gaggtgcagctggtggagtccgggggaggcgtggtccagcctgggaggtccctgagactctcctgtgcagcctctggattcaccttcagtagctatgctatgcactgggtccgccaggctccaggacaagggcttgagtgggtctcaactattggtcctagaggcattaacacgcactacgcagactcagtgaagggccgattcaccatctccagagacaattccaagaacacgctgtatctgcaaatgaacagcctgagagccgaggacacagccacatattactgtgcgagagatcgggttggctacaattcctacggtatggacgtctggggccaagggacaatggtcaccgtctcttca

SEQ ID NO: 546 - DNA, 363 bp, synthetic construct
  source 1..363: mol_type = other DNA
gaggtgcagctggtggagtccgggggaggcgtggtccagcctgggaggtccctgagactctcctgtgcagcctctggattcaccttcagtagctatgctatgcactgggtccgccaggctccagggaaggggctggagtggattggggaaatctatcatggtgggagcaccgactacaacccgtccctcaagagtcgagtcaccatctccagagacgattccacgaacacgctgtatctgcaaatgaacagcctgagagccgaggacacagccgtgtattactgtacaagaggcgggaagaggctattgtccgtctatggtgtagacgtctggggccaagggacaatggtcaccgtctcttca

SEQ ID NO: 547 - protein, 5 aa, synthetic construct
SYAMS

SEQ ID NO: 548 - protein, 5 aa, synthetic construct
SYAMD

SEQ ID NO: 549 - protein, 5 aa, synthetic construct
SYEMS

SEQ ID NO: 550 - protein, 5 aa, synthetic construct
SYEMD

SEQ ID NO: 551 - protein, 5 aa, synthetic construct
SYEMH

SEQ ID NO: 552 - protein, 5 aa, synthetic construct
TYAMS

SEQ ID NO: 553 - protein, 16 aa, synthetic construct
GGGGGSGGGGSGGGGS

SEQ ID NO: 554 - protein, 26 aa, synthetic construct
GGGGGSGGGGSGGGGSGGGGSGGGGS

SEQ ID NO: 555 - protein, 25 aa, synthetic construct
GGGGSGGGGSGGGGSGGGGSGGGGS

SEQ ID NO: 556 - skipped

SEQ ID NO: 557 - protein, 18 aa, synthetic construct
VEGGSGGSGGSGGSGGVD

SEQ ID NO: 558 - protein, 22 aa, synthetic construct
MDMRVPAQLLGLLLLWLRGARC

SEQ ID NO: 559 - DNA, 66 bp, synthetic construct
  source 1..66: mol_type = other DNA
atggatatgagggtccccgcacaattgctcggtctccttctgctttggctccggggcgcgcggtgc

SEQ ID NO: 560 - protein, 22 aa, synthetic construct
MDMRVLAQLLGLLLLCFPGARC

SEQ ID NO: 561 - protein, 20 aa, synthetic construct
MEAPAQLLFLLLLWLPDTTG

SEQ ID NO: 562 - protein, 20 aa, synthetic construct
MVLQTQVFISLLLWISGAYG

SEQ ID NO: 563 - protein, 19 aa, synthetic construct
MLPSQLIGFLLLWVPASRG

SEQ ID NO: 564 - protein, 22 aa, synthetic construct
MDMRVPAQLLGLLLLWLSGARC

SEQ ID NO: 565 - protein, 5 aa, synthetic construct
TYAMD

SEQ ID NO: 566 - skipped

SEQ ID NO: 567 - protein, 5 aa, synthetic construct
TYEMS

SEQ ID NO: 568 - protein, 5 aa, synthetic construct
SYEMD

SEQ ID NO: 569 - protein, 5 aa, synthetic construct
TYEMD

SEQ ID NO: 570 - protein, 16 aa, synthetic construct
SIGPRGINTHYADSVK

SEQ ID NO: 571 - protein, 16 aa, synthetic construct
TIGPKGINTHYADSVK

SEQ ID NO: 572 - protein, 16 aa, synthetic construct
TIGPRGINSHYADSVK

SEQ ID NO: 573 - protein, 16 aa, synthetic construct
TIGPRGINTHYAESVK

SEQ ID NO: 574 - protein, 16 aa, synthetic construct
TIGPRGINTHYADTVK

SEQ ID NO: 575 - protein, 16 aa, synthetic construct
TIGPRGINTHYADSVR

SEQ ID NO: 576 - protein, 16 aa, synthetic construct
TIGPRGINTHYADSLK

SEQ ID NO: 577 - protein, 16 aa, synthetic construct
TLGPRGINTHYADSVK

SEQ ID NO: 578 - protein, 16 aa, synthetic construct
SIGPRGINTHYADTVK

SEQ ID NO: 579 - protein, 16 aa, synthetic construct
SIGPRGINTHYADTVR

SEQ ID NO: 580 - protein, 12 aa, synthetic construct
ERVGYNSYGMDV

SEQ ID NO: 581 - protein, 12 aa, synthetic construct
DKVGYNSYGMDV

SEQ ID NO: 582 - protein, 12 aa, synthetic construct
DRAGYNSYGMDV

SEQ ID NO: 583 - protein, 12 aa, synthetic construct
DRVGYQSYGMDV

SEQ ID NO: 584 - protein, 12 aa, synthetic construct
DRVGYNTYGMDV

SEQ ID NO: 585 - protein, 12 aa, synthetic construct
DRVGYNSYGMEV

SEQ ID NO: 586 - protein, 12 aa, synthetic construct
DRVGYNSYGMDL

SEQ ID NO: 587 - protein, 12 aa, synthetic construct
DKVGYNTYGMDV

SEQ ID NO: 588 - protein, 12 aa, synthetic construct
DRVGYNTYGMEV

SEQ ID NO: 589 - protein, 12 aa, synthetic construct
DRVGYNTYGMDA

SEQ ID NO: 590 - protein, 5 aa, synthetic construct
SYAMS

SEQ ID NO: 591 - protein, 5 aa, synthetic construct
SYAMD

SEQ ID NO: 592 - protein, 5 aa, synthetic construct
SYEMS

SEQ ID NO: 593 - protein, 5 aa, synthetic construct
SYEMD

SEQ ID NO: 594 - protein, 5 aa, synthetic construct
SYEMH

SEQ ID NO: 595 - protein, 5 aa, synthetic construct
TYAMS

SEQ ID NO: 596 - protein, 5 aa, synthetic construct
TYAMD

SEQ ID NO: 597 - protein, 5 aa, synthetic construct
TYEMS

SEQ ID NO: 598 - protein, 5 aa, synthetic construct
SYEMD

SEQ ID NO: 599 - protein, 5 aa, synthetic construct
TYEMD

SEQ ID NO: 600 - protein, 15 aa, synthetic construct
DIYHGGSTDYNPSLK

SEQ ID NO: 601 - protein, 15 aa, synthetic construct
ELYHGGSTDYNPSLK

SEQ ID NO: 602 - protein, 15 aa, synthetic construct
EIYDGGSTDYNPSLK

SEQ ID NO: 603 - protein, 15 aa, synthetic construct
EIYHGGTTDYNPSLK

SEQ ID NO: 604 - protein, 15 aa, synthetic construct
EIYHGGSSDYNPSLK

SEQ ID NO: 605 - protein, 15 aa, synthetic construct
EIYHGGSTEYNPSLK

SEQ ID NO: 606 - protein, 15 aa, synthetic construct
EIYHGGSTDYQPSLK

SEQ ID NO: 607 - protein, 15 aa, synthetic construct
EIYHGGSTDYNPTLK

SEQ ID NO: 608 - protein, 15 aa, synthetic construct
EIYHGGSTDYNPSIK

SEQ ID NO: 609 - protein, 15 aa, synthetic construct
EIYHGGSTDYNPSLR

SEQ ID NO: 610 - protein, 13 aa, synthetic construct
GGRRLLSVYGVDV

SEQ ID NO: 611 - protein, 13 aa, synthetic construct
GGKKLLSVYGVDV

SEQ ID NO: 612 - protein, 13 aa, synthetic construct
GGRKLLSVYGVDV

SEQ ID NO: 613 - protein, 13 aa, synthetic construct
GGKRLLTVYGVDV

SEQ ID NO: 614 - protein, 13 aa, synthetic construct
GGKRLLSVYGVEV

SEQ ID NO: 615 - protein, 13 aa, synthetic construct
GGKRLLSVYGVDL

SEQ ID NO: 616 - protein, 13 aa, synthetic construct
GGKRLLSVYGLDV

SEQ ID NO: 617 - protein, 13 aa, synthetic construct
GGKRLLTVYGVEV

SEQ ID NO: 618 - protein, 13 aa, synthetic construct
GGKKLLSVYGVEV

SEQ ID NO: 619 - protein, 13 aa, synthetic construct
GGRRLLSVYGVEV